SEQUENCE LISTING (204 sequences)

SEQ ID NO: 1 - DNA, 36 bp, Homo sapiens
ggcctcccaataaagctggacaagaagctgctatga

SEQ ID NO: 2 - DNA, 20 bp, Homo sapiens
ggtgcgaagcagactgaggc

SEQ ID NO: 3 - DNA, 20 bp, Homo sapiens
gggatttcatgtaaccaaga

SEQ ID NO: 4 - DNA, 16 bp, Homo sapiens
gctgacatccaaatag

SEQ ID NO: 5 - DNA, 21 bp, Homo sapiens
ccagtggattctgtgttgttt

SEQ ID NO: 6 - DNA, 20 bp, Homo sapiens
agcttcttgtccagctttat

SEQ ID NO: 7 - DNA, 20 bp, Homo sapiens
gcctcagtctgcttcgcacc

SEQ ID NO: 8 - DNA, 20 bp, Homo sapiens
tcttggttacatgaaatccc

SEQ ID NO: 9 - DNA, 16 bp, Homo sapiens
ctatttggatgtcagc

SEQ ID NO: 10 - DNA, 21 bp, Homo sapiens
aaacaacacagaatccactgg

SEQ ID NO: 11 - DNA, 20 bp, Artificial Sequence
  Artificially synthesized oligonucleotide sequence
agcuucttgtccagcuuuau

SEQ ID NO: 12 - DNA, 10 bp, Artificial Sequence
  Artificially synthesized oligonucleotide sequence
acaagaagcu

SEQ ID NO: 13 - DNA, 11 bp, Artificial Sequence
  Artificially synthesized oligonucleotide sequence
gacaagaagcu

SEQ ID NO: 14 - DNA, 12 bp, Artificial Sequence
  Artificially synthesized oligonucleotide sequence
ggacaagaagcu

SEQ ID NO: 15 - DNA, 13 bp, Artificial Sequence
  Artificially synthesized oligonucleotide sequence
tggacaagaagcu

SEQ ID NO: 16 - DNA, 14 bp, Artificial Sequence
  Artificially synthesized oligonucleotide sequence
ctggacaagaagcu

SEQ ID NO: 17 - DNA, 15 bp, Artificial Sequence
  Artificially synthesized oligonucleotide sequence
gctggacaagaagcu

SEQ ID NO: 18 - DNA, 16 bp, Artificial Sequence
  Artificially synthesized oligonucleotide sequence
agctggacaagaagcu

SEQ ID NO: 19 - DNA, 17 bp, Artificial Sequence
  Artificially synthesized oligonucleotide sequence
aagctggacaagaagcu

SEQ ID NO: 20 - DNA, 18 bp, Artificial Sequence
  Artificially synthesized oligonucleotide sequence
aaagctggacaagaagcu

SEQ ID NO: 21 - DNA, 19 bp, Artificial Sequence
  Artificially synthesized oligonucleotide sequence
uaaagctggacaagaagcu

SEQ ID NO: 22 - DNA, 19 bp, Artificial Sequence
  Artificially synthesized oligonucleotide sequence
auaaagctggacaagaagc

SEQ ID NO: 23 - DNA, 18 bp, Artificial Sequence
  Artificially synthesized oligonucleotide sequence
auaaagctggacaagaag

SEQ ID NO: 24 - DNA, 17 bp, Artificial Sequence
  Artificially synthesized oligonucleotide sequence
auaaagctggacaagaa

SEQ ID NO: 25 - DNA, 16 bp, Artificial Sequence
  Artificially synthesized oligonucleotide sequence
auaaagctggacaaga

SEQ ID NO: 26 - DNA, 15 bp, Artificial Sequence
  Artificially synthesized oligonucleotide sequence
auaaagctggacaag

SEQ ID NO: 27 - DNA, 14 bp, Artificial Sequence
  Artificially synthesized oligonucleotide sequence
auaaagctggacaa

SEQ ID NO: 28 - DNA, 13 bp, Artificial Sequence
  Artificially synthesized oligonucleotide sequence
auaaagctggaca

SEQ ID NO: 29 - DNA, 12 bp, Artificial Sequence
  Artificially synthesized oligonucleotide sequence
auaaagctggac

SEQ ID NO: 30 - DNA, 11 bp, Artificial Sequence
  Artificially synthesized oligonucleotide sequence
auaaagctgga

SEQ ID NO: 31 - DNA, 10 bp, Artificial Sequence
  Artificially synthesized oligonucleotide sequence
auaaagctgg

SEQ ID NO: 32 - DNA, 18 bp, Artificial Sequence
  Artificially synthesized oligonucleotide sequence
uaaagctggacaagaagc

SEQ ID NO: 33 - DNA, 17 bp, Artificial Sequence
  Artificially synthesized oligonucleotide sequence
aaagctggacaagaagc

SEQ ID NO: 34 - DNA, 17 bp, Artificial Sequence
  Artificially synthesized oligonucleotide sequence
uaaagctggacaagaag

SEQ ID NO: 35 - DNA, 16 bp, Artificial Sequence
  Artificially synthesized oligonucleotide sequence
aaagctggacaagaag

SEQ ID NO: 36 - DNA, 15 bp, Artificial Sequence
  Artificially synthesized oligonucleotide sequence
aagctggacaagaag

SEQ ID NO: 37 - DNA, 15 bp, Artificial Sequence
  Artificially synthesized oligonucleotide sequence
aaagctggacaagaa

SEQ ID NO: 38 - DNA, 14 bp, Artificial Sequence
  Artificially synthesized oligonucleotide sequence
aagctggacaagaa

SEQ ID NO: 39 - DNA, 13 bp, Artificial Sequence
  Artificially synthesized oligonucleotide sequence
agctggacaagaa

SEQ ID NO: 40 - DNA, 13 bp, Artificial Sequence
  Artificially synthesized oligonucleotide sequence
aagctggacaaga

SEQ ID NO: 41 - DNA, 12 bp, Artificial Sequence
  Artificially synthesized oligonucleotide sequence
agctggacaaga

SEQ ID NO: 42 - DNA, 11 bp, Artificial Sequence
  Artificially synthesized oligonucleotide sequence
gctggacaaga

SEQ ID NO: 43 - DNA, 11 bp, Artificial Sequence
  Artificially synthesized oligonucleotide sequence
agctggacaag

SEQ ID NO: 44 - DNA, 10 bp, Artificial Sequence
  Artificially synthesized oligonucleotide sequence
gctggacaag

SEQ ID NO: 45 - DNA, 10 bp, Artificial Sequence
  Artificially synthesized oligonucleotide sequence
acaagaagct

SEQ ID NO: 46 - DNA, 11 bp, Artificial Sequence
  Artificially synthesized oligonucleotide sequence
gacaagaagct

SEQ ID NO: 47 - DNA, 12 bp, Artificial Sequence
  Artificially synthesized oligonucleotide sequence
ggacaagaagct

SEQ ID NO: 48 - DNA, 13 bp, Artificial Sequence
  Artificially synthesized oligonucleotide sequence
tggacaagaagct

SEQ ID NO: 49 - DNA, 14 bp, Artificial Sequence
  Artificially synthesized oligonucleotide sequence
ctggacaagaagct

SEQ ID NO: 50 - DNA, 15 bp, Artificial Sequence
  Artificially synthesized oligonucleotide sequence
gctggacaagaagct

SEQ ID NO: 51 - DNA, 16 bp, Artificial Sequence
  Artificially synthesized oligonucleotide sequence
agctggacaagaagct

SEQ ID NO: 52 - DNA, 17 bp, Artificial Sequence
  Artificially synthesized oligonucleotide sequence
aagctggacaagaagct

SEQ ID NO: 53 - DNA, 18 bp, Artificial Sequence
  Artificially synthesized oligonucleotide sequence
aaagctggacaagaagct

SEQ ID NO: 54 - DNA, 19 bp, Artificial Sequence
  Artificially synthesized oligonucleotide sequence
taaagctggacaagaagct

SEQ ID NO: 55 - DNA, 19 bp, Artificial Sequence
  Artificially synthesized oligonucleotide sequence
ataaagctggacaagaagc

SEQ ID NO: 56 - DNA, 18 bp, Artificial Sequence
  Artificially synthesized oligonucleotide sequence
ataaagctggacaagaag

SEQ ID NO: 57 - DNA, 17 bp, Artificial Sequence
  Artificially synthesized oligonucleotide sequence
ataaagctggacaagaa

SEQ ID NO: 58 - DNA, 16 bp, Artificial Sequence
  Artificially synthesized oligonucleotide sequence
ataaagctggacaaga

SEQ ID NO: 59 - DNA, 15 bp, Artificial Sequence
  Artificially synthesized oligonucleotide sequence
ataaagctggacaag

SEQ ID NO: 60 - DNA, 14 bp, Artificial Sequence
  Artificially synthesized oligonucleotide sequence
ataaagctggacaa

SEQ ID NO: 61 - DNA, 13 bp, Artificial Sequence
  Artificially synthesized oligonucleotide sequence
ataaagctggaca

SEQ ID NO: 62 - DNA, 12 bp, Artificial Sequence
  Artificially synthesized oligonucleotide sequence
ataaagctggac

SEQ ID NO: 63 - DNA, 18 bp, Artificial Sequence
  Artificially synthesized oligonucleotide sequence
taaagctggacaagaagc

SEQ ID NO: 64 - DNA, 17 bp, Artificial Sequence
  Artificially synthesized oligonucleotide sequence
aaagctggacaagaagc

SEQ ID NO: 65 - DNA, 17 bp, Artificial Sequence
  Artificially synthesized oligonucleotide sequence
taaagctggacaagaag

SEQ ID NO: 66 - DNA, 16 bp, Artificial Sequence
  Artificially synthesized oligonucleotide sequence
aaagctggacaagaag

SEQ ID NO: 67 - DNA, 15 bp, Artificial Sequence
  Artificially synthesized oligonucleotide sequence
aagctggacaagaag

SEQ ID NO: 68 - DNA, 15 bp, Artificial Sequence
  Artificially synthesized oligonucleotide sequence
aaagctggacaagaa

SEQ ID NO: 69 - DNA, 14 bp, Artificial Sequence
  Artificially synthesized oligonucleotide sequence
aagctggacaagaa

SEQ ID NO: 70 - DNA, 13 bp, Artificial Sequence
  Artificially synthesized oligonucleotide sequence
agctggacaagaa

SEQ ID NO: 71 - DNA, 13 bp, Artificial Sequence
  Artificially synthesized oligonucleotide sequence
aagctggacaaga

SEQ ID NO: 72 - DNA, 12 bp, Artificial Sequence
  Artificially synthesized oligonucleotide sequence
agctggacaaga

SEQ ID NO: 73 - DNA, 11 bp, Artificial Sequence
  Artificially synthesized oligonucleotide sequence
gctggacaaga

SEQ ID NO: 74 - DNA, 11 bp, Artificial Sequence
  Artificially synthesized oligonucleotide sequence
agctggacaag

SEQ ID NO: 75 - DNA, 10 bp, Artificial Sequence
  Artificially synthesized oligonucleotide sequence
gctggacaag

SEQ ID NO: 76 - DNA, 10 bp, Artificial Sequence
  Artificially synthesized oligonucleotide sequence
acaagaagct

SEQ ID NO: 77 - DNA, 11 bp, Artificial Sequence
  Artificially synthesized oligonucleotide sequence
gacaagaagct

SEQ ID NO: 78 - DNA, 12 bp, Artificial Sequence
  Artificially synthesized oligonucleotide sequence
ggacaagaagct

SEQ ID NO: 79 - DNA, 13 bp, Artificial Sequence
  Artificially synthesized oligonucleotide sequence
tggacaagaagct

SEQ ID NO: 80 - DNA, 14 bp, Artificial Sequence
  Artificially synthesized oligonucleotide sequence
ctggacaagaagct

SEQ ID NO: 81 - DNA, 15 bp, Artificial Sequence
  Artificially synthesized oligonucleotide sequence
gctggacaagaagct

SEQ ID NO: 82 - DNA, 16 bp, Artificial Sequence
  Artificially synthesized oligonucleotide sequence
agctggacaagaagct

SEQ ID NO: 83 - DNA, 17 bp, Artificial Sequence
  Artificially synthesized oligonucleotide sequence
aagctggacaagaagct

SEQ ID NO: 84 - DNA, 18 bp, Artificial Sequence
  Artificially synthesized oligonucleotide sequence
aaagctggacaagaagct

SEQ ID NO: 85 - DNA, 19 bp, Artificial Sequence
  Artificially synthesized oligonucleotide sequence
taaagctggacaagaagct

SEQ ID NO: 86 - DNA, 19 bp, Artificial Sequence
  Artificially synthesized oligonucleotide sequence
ataaagctggacaagaagc

SEQ ID NO: 87 - DNA, 18 bp, Artificial Sequence
  Artificially synthesized oligonucleotide sequence
ataaagctggacaagaag

SEQ ID NO: 88 - DNA, 17 bp, Artificial Sequence
  Artificially synthesized oligonucleotide sequence
ataaagctggacaagaa

SEQ ID NO: 89 - DNA, 16 bp, Artificial Sequence
  Artificially synthesized oligonucleotide sequence
ataaagctggacaaga

SEQ ID NO: 90 - DNA, 15 bp, Artificial Sequence
  Artificially synthesized oligonucleotide sequence
ataaagctggacaag

SEQ ID NO: 91 - DNA, 14 bp, Artificial Sequence
  Artificially synthesized oligonucleotide sequence
ataaagctggacaa

SEQ ID NO: 92 - DNA, 13 bp, Artificial Sequence
  Artificially synthesized oligonucleotide sequence
ataaagctggaca

SEQ ID NO: 93 - DNA, 12 bp, Artificial Sequence
  Artificially synthesized oligonucleotide sequence
ataaagctggac

SEQ ID NO: 94 - DNA, 11 bp, Artificial Sequence
  Artificially synthesized oligonucleotide sequence
ataaagctgga

SEQ ID NO: 95 - DNA, 10 bp, Artificial Sequence
  Artificially synthesized oligonucleotide sequence
ataaagctgg

SEQ ID NO: 96 - DNA, 18 bp, Artificial Sequence
  Artificially synthesized oligonucleotide sequence
taaagctggacaagaagc

SEQ ID NO: 97 - DNA, 17 bp, Artificial Sequence
  Artificially synthesized oligonucleotide sequence
aaagctggacaagaagc

SEQ ID NO: 98 - DNA, 17 bp, Artificial Sequence
  Artificially synthesized oligonucleotide sequence
taaagctggacaagaag

SEQ ID NO: 99 - DNA, 16 bp, Artificial Sequence
  Artificially synthesized oligonucleotide sequence
aaagctggacaagaag

SEQ ID NO: 100 - DNA, 15 bp, Artificial Sequence
  Artificially synthesized oligonucleotide sequence
aagctggacaagaag

SEQ ID NO: 101 - DNA, 15 bp, Artificial Sequence
  Artificially synthesized oligonucleotide sequence
aaagctggacaagaa

SEQ ID NO: 102 - DNA, 14 bp, Artificial Sequence
  Artificially synthesized oligonucleotide sequence
aagctggacaagaa

SEQ ID NO: 103 - DNA, 13 bp, Artificial Sequence
  Artificially synthesized oligonucleotide sequence
agctggacaagaa

SEQ ID NO: 104 - DNA, 13 bp, Artificial Sequence
  Artificially synthesized oligonucleotide sequence
aagctggacaaga

SEQ ID NO: 105 - DNA, 12 bp, Artificial Sequence
  Artificially synthesized oligonucleotide sequence
agctggacaaga

SEQ ID NO: 106 - DNA, 11 bp, Artificial Sequence
  Artificially synthesized oligonucleotide sequence
gctggacaaga

SEQ ID NO: 107 - DNA, 11 bp, Artificial Sequence
  Artificially synthesized oligonucleotide sequence
agctggacaag

SEQ ID NO: 108 - DNA, 10 bp, Artificial Sequence
  Artificially synthesized oligonucleotide sequence
gctggacaag

SEQ ID NO: 109 - DNA, 20 bp, Artificial Sequence
  Artificially synthesized oligonucleotide sequence
agcutcttgtccagcuuuau

SEQ ID NO: 110 - DNA, 20 bp, Artificial Sequence
  Artificially synthesized oligonucleotide sequence
agcuucttgtccagctuuau

SEQ ID NO: 111 - DNA, 20 bp, Artificial Sequence
  Artificially synthesized oligonucleotide sequence
agcutcttgtccagctuuau

SEQ ID NO: 112 - DNA, 20 bp, Artificial Sequence
  Artificially synthesized oligonucleotide sequence
agcttcttgtccagctuuau

SEQ ID NO: 113 - DNA, 20 bp, Artificial Sequence
  Artificially synthesized oligonucleotide sequence
agcttcttgtccagcttuau

SEQ ID NO: 114 - DNA, 20 bp, Artificial Sequence
  Artificially synthesized oligonucleotide sequence
agcttcttgtccagctttau

SEQ ID NO: 115 - DNA, 20 bp, Artificial Sequence
  Artificially synthesized oligonucleotide sequence
agcttcttgtccagcttuau

SEQ ID NO: 116 - DNA, 20 bp, Artificial Sequence
  Artificially synthesized oligonucleotide sequence
agcttcttgtccagctttau

SEQ ID NO: 117 - DNA, 20 bp, Artificial Sequence
  Artificially synthesized oligonucleotide sequence
agcttcttgtccagctttau

SEQ ID NO: 118 - DNA, 20 bp, Artificial Sequence
  Artificially synthesized oligonucleotide sequence
agcttcttgtccagctttau

SEQ ID NO: 119 - DNA, 20 bp, Artificial Sequence
  Artificially synthesized oligonucleotide sequence
agcttcttgtccagctttau

SEQ ID NO: 120 - DNA, 20 bp, Artificial Sequence
  Artificially synthesized oligonucleotide sequence
agcttcttgtccagctttat

SEQ ID NO: 121 - DNA, 20 bp, Artificial Sequence
  Artificially synthesized oligonucleotide sequence
agcttcttgtccagctttau

SEQ ID NO: 122 - DNA, 20 bp, Artificial Sequence
  Artificially synthesized oligonucleotide sequence
agcttcttgtccagctttat

SEQ ID NO: 123 - DNA, 20 bp, Artificial Sequence
  Artificially synthesized oligonucleotide sequence
agcuucuuguccagctttat

SEQ ID NO: 124 - DNA, 20 bp, Artificial Sequence
  Artificially synthesized oligonucleotide sequence
agcuucuugtccagctttau

SEQ ID NO: 125 - DNA, 20 bp, Artificial Sequence
  Artificially synthesized oligonucleotide sequence
agcuucuugtccagctttau

SEQ ID NO: 126 - DNA, 20 bp, Artificial Sequence
  Artificially synthesized oligonucleotide sequence
agcuucutgtccagcttuau

SEQ ID NO: 127 - DNA, 20 bp, Artificial Sequence
  Artificially synthesized oligonucleotide sequence
agcuucttgtccagctuuau

SEQ ID NO: 128 - DNA, 20 bp, Artificial Sequence
  Artificially synthesized oligonucleotide sequence
agcutcttgtccagcuuuau

SEQ ID NO: 129 - DNA, 20 bp, Artificial Sequence
  Artificially synthesized oligonucleotide sequence
agcttcttgtccagcuuuau

SEQ ID NO: 130 - DNA, 20 bp, Artificial Sequence
  Artificially synthesized oligonucleotide sequence
agcttcttgtccagcuuuau

SEQ ID NO: 131 - DNA, 20 bp, Artificial Sequence
  Artificially synthesized oligonucleotide sequence
agcttcttgtccagcuuuau

SEQ ID NO: 132 - DNA, 20 bp, Artificial Sequence
  Artificially synthesized oligonucleotide sequence
agcttcttgtccagcuuuau

SEQ ID NO: 133 - DNA, 19 bp, Artificial Sequence
  Artificially synthesized oligonucleotide sequence
gcuucttgtccagcuuuau

SEQ ID NO: 134 - DNA, 19 bp, Artificial Sequence
  Artificially synthesized oligonucleotide sequence
agcuucttgtccagcuuua

SEQ ID NO: 135 - DNA, 18 bp, Artificial Sequence
  Artificially synthesized oligonucleotide sequence
gcuucttgtccagcuuua

SEQ ID NO: 136 - DNA, 17 bp, Artificial Sequence
  Artificially synthesized oligonucleotide sequence
cuucttgtccagcuuua

SEQ ID NO: 137 - DNA, 17 bp, Artificial Sequence
  Artificially synthesized oligonucleotide sequence
gcuucttgtccagcuuu

SEQ ID NO: 138 - DNA, 16 bp, Artificial Sequence
  Artificially synthesized oligonucleotide sequence
cuucttgtccagcuuu

SEQ ID NO: 139 - DNA, 15 bp, Artificial Sequence
  Artificially synthesized oligonucleotide sequence
uucttgtccagcuuu

SEQ ID NO: 140 - DNA, 15 bp, Artificial Sequence
  Artificially synthesized oligonucleotide sequence
cuucttgtccagcuu

SEQ ID NO: 141 - DNA, 14 bp, Artificial Sequence
  Artificially synthesized oligonucleotide sequence
uucttgtccagcuu

SEQ ID NO: 142 - DNA, 20 bp, Artificial Sequence
  Artificially synthesized oligonucleotide sequence
gccucagtctgcttcgcacc

SEQ ID NO: 143 - DNA, 15 bp, Artificial Sequence
  Artificially synthesized oligonucleotide sequence
ugcgaagcagactga

SEQ ID NO: 144 - DNA, 13 bp, Artificial Sequence
  Artificially synthesized oligonucleotide sequence
gcgaagcagactg

SEQ ID NO: 145 - DNA, 20 bp, Artificial Sequence
  Artificially synthesized oligonucleotide sequence
ucuuggttacatgaaauccc

SEQ ID NO: 146 - DNA, 13 bp, Artificial Sequence
  Artificially synthesized oligonucleotide sequence
catgtaaccaaga

SEQ ID NO: 147 - DNA, 13 bp, Artificial Sequence
  Artificially synthesized oligonucleotide sequence
gggauttcatgta

SEQ ID NO: 148 - DNA, 15 bp, Artificial Sequence
  Artificially synthesized oligonucleotide sequence
auttcatgtaaccaa

SEQ ID NO: 149 - DNA, 15 bp, Artificial Sequence
  Artificially synthesized oligonucleotide sequence
gauttcatgtaacca

SEQ ID NO: 150 - DNA, 13 bp, Artificial Sequence
  Artificially synthesized oligonucleotide sequence
auttcatgtaacc

SEQ ID NO: 151 - DNA, 16 bp, Artificial Sequence
  Artificially synthesized oligonucleotide sequence
ctatttggatgtcagc

SEQ ID NO: 152 - DNA, 11 bp, Artificial Sequence
  Artificially synthesized oligonucleotide sequence
gcugacatcca

SEQ ID NO: 153 - DNA, 13 bp, Artificial Sequence
  Artificially synthesized oligonucleotide sequence
cugacatccaaau

SEQ ID NO: 154 - DNA, 14 bp, Artificial Sequence
  Artificially synthesized oligonucleotide sequence
cugacatccaaaua

SEQ ID NO: 155 - DNA, 21 bp, Artificial Sequence
  Artificially synthesized oligonucleotide sequence
aaacaacacagaatccacugg

SEQ ID NO: 156 - DNA, 15 bp, Artificial Sequence
  Artificially synthesized oligonucleotide sequence
guggattctgtguug

SEQ ID NO: 157 - DNA, 20 bp, Artificial Sequence
  Artificially synthesized oligonucleotide sequence
agcuucttgtccagcuuuau

SEQ ID NO: 158 - DNA, 20 bp, Artificial Sequence
  Artificially synthesized oligonucleotide sequence
agcuucttgtccagcuuuau

SEQ ID NO: 159 - DNA, 20 bp, Artificial Sequence
  Artificially synthesized oligonucleotide sequence
agcuucttgtccagcuuuau

SEQ ID NO: 160 - DNA, 20 bp, Artificial Sequence
  Artificially synthesized oligonucleotide sequence
agcuucttgtccagcuuuau

SEQ ID NO: 161 - DNA, 20 bp, Artificial Sequence
  Artificially synthesized oligonucleotide sequence
agcuucutgtccagcuuuau

SEQ ID NO: 162 - DNA, 16 bp, Artificial Sequence
  Artificially synthesized oligonucleotide sequence
cuucttgtccagcuuu

SEQ ID NO: 163 - DNA, 14 bp, Artificial Sequence
  Artificially synthesized oligonucleotide sequence
uucttgtccagcuu

SEQ ID NO: 164-165 - skipped (2 entries)

SEQ ID NO: 166 - DNA, 10 bp, Artificial Sequence
  Artificially synthesized oligonucleotide sequence
cttgtccagc

SEQ ID NO: 167 - DNA, 12 bp, Artificial Sequence
  Artificially synthesized oligonucleotide sequence
ucttgtccagcu

SEQ ID NO: 168 - DNA, 20 bp, Artificial Sequence
  Artificially synthesized oligonucleotide sequence
ataaagctggacaagaagct

SEQ ID NO: 169 - DNA, 24 bp, Artificial Sequence
  Artificially synthesized oligonucleotide sequence
gcagcutcttgtccagctuuauug

SEQ ID NO: 170 - DNA, 28 bp, Artificial Sequence
  Artificially synthesized oligonucleotide sequence
uagcagcttcttgtccagcttuauuggg

SEQ ID NO: 171 - DNA, 32 bp, Artificial Sequence
  Artificially synthesized oligonucleotide sequence
cauagcagcttcttgtccagctttauugggag

SEQ ID NO: 172 - DNA, 36 bp, Artificial Sequence
  Artificially synthesized oligonucleotide sequence
ucauagcagcttcttgtccagctttauugggaggcc

SEQ ID NO: 173 - DNA, 24 bp, Artificial Sequence
  Artificially synthesized oligonucleotide sequence
caataaagctggacaagaagctgc

SEQ ID NO: 174 - DNA, 28 bp, Artificial Sequence
  Artificially synthesized oligonucleotide sequence
cccaataaagctggacaagaagctgcta

SEQ ID NO: 175 - DNA, 32 bp, Artificial Sequence
  Artificially synthesized oligonucleotide sequence
ctcccaataaagctggacaagaagctgctatg

SEQ ID NO: 176-177 - skipped (2 entries)

SEQ ID NO: 178 - DNA, 12 bp, Artificial Sequence
  Artificially synthesized oligonucleotide sequence
caataaagctgg

SEQ ID NO: 179 - DNA, 12 bp, Artificial Sequence
  Artificially synthesized oligonucleotide sequence
cccaataaagct

SEQ ID NO: 180 - DNA, 12 bp, Artificial Sequence
  Artificially synthesized oligonucleotide sequence
ctcccaataaag

SEQ ID NO: 181 - DNA, 12 bp, Artificial Sequence
  Artificially synthesized oligonucleotide sequence
ggcctcccaata

SEQ ID NO: 182 - DNA, 20 bp, Artificial Sequence
  Artificially synthesized oligonucleotide sequence
agcttcttgtccagcttuau

SEQ ID NO: 183 - DNA, 20 bp, Artificial Sequence
  Artificially synthesized oligonucleotide sequence
agctucttgtccagcutuau

SEQ ID NO: 184 - DNA, 20 bp, Artificial Sequence
  Artificially synthesized oligonucleotide sequence
agcuucttgtccagctutau

SEQ ID NO: 185 - DNA, 20 bp, Artificial Sequence
  Artificially synthesized oligonucleotide sequence
agcuucutgtccagctutau

SEQ ID NO: 186 - DNA, 20 bp, Artificial Sequence
  Artificially synthesized oligonucleotide sequence
agcttcttgtccagctttau

SEQ ID NO: 187 - DNA, 20 bp, Artificial Sequence
  Artificially synthesized oligonucleotide sequence
agcuucttctccagcuuuau

SEQ ID NO: 188 - DNA, 16 bp, Artificial Sequence
  Artificially synthesized oligonucleotide sequence
aaagctggagaagaag

SEQ ID NO: 189 - DNA, 20 bp, Artificial Sequence
  Artificially synthesized oligonucleotide sequence
agcuugttgtccagcuauau

SEQ ID NO: 190 - DNA, 16 bp, Artificial Sequence
  Artificially synthesized oligonucleotide sequence
atagctggacaacaag

SEQ ID NO: 191 - DNA, 20 bp, Artificial Sequence
  Artificially synthesized oligonucleotide sequence
agguucatgtccagauuuau

SEQ ID NO: 192 - DNA, 16 bp, Artificial Sequence
  Artificially synthesized oligonucleotide sequence
aaatctggacatgaag

SEQ ID NO: 193 - DNA, 20 bp, Artificial Sequence
  Artificially synthesized oligonucleotide sequence
uucuatttggatgtcagcaa

SEQ ID NO: 194 - DNA, 16 bp, Artificial Sequence
  Artificially synthesized oligonucleotide sequence
gctgacatccaaatag

SEQ ID NO: 195 - RNA, 20 nt, Artificial Sequence
  Artificially synthesized oligonucleotide sequence
uucuauuuggaugucagcaa

SEQ ID NO: 196 - RNA, 20 nt, Artificial Sequence
  Artificially synthesized oligonucleotide sequence
gcugacauccaaauagaauu

SEQ ID NO: 197 - DNA, 10 bp, Artificial Sequence
  Artificially synthesized oligonucleotide sequence
cttgtccagc

SEQ ID NO: 198 - DNA, 12 bp, Artificial Sequence
  Artificially synthesized oligonucleotide sequence
ucttgtccagcu

SEQ ID NO: 199 - DNA, 14 bp, Artificial Sequence
  Artificially synthesized oligonucleotide sequence
uucttgtccagcuu

SEQ ID NO: 200 - DNA, 16 bp, Artificial Sequence
  Artificially synthesized oligonucleotide sequence
taagcaggacaacaag

SEQ ID NO: 201 - DNA, 20 bp, Artificial Sequence
  Artificially synthesized oligonucleotide sequence
agcuucutgtccagcuuuau

SEQ ID NO: 202 - DNA, 20 bp, Artificial Sequence
  Artificially synthesized oligonucleotide sequence
agcuucutgtccagcuuuau

SEQ ID NO: 203 - DNA, 20 bp, Artificial Sequence
  Artificially synthesized oligonucleotide sequence
agcuucuugtccagcuuuau

SEQ ID NO: 204 - DNA, 20 bp, Artificial Sequence
  Artificially synthesized oligonucleotide sequence
agcuucuugtccagcuuuau

(19) United States
(12) Patent Application Publication
(10) Pub. No.: US 2024/0271137 A1
(43) Pub. Date: Aug. 15, 2024

(54) Asymmetric Short Duplex DNA as a Novel Gene Silencing Technology and Use Thereof

(71) Applicant: 1Globe Health Institute LLC, Norwood, MA (US)

(72) Inventors: Chiang J. LI, Cambridge, MA (US); Xiangao SUN, Norwood, MA (US); Charles LI, Boston, MA (US)

(21) Appl. No.: 18/564,562

(22) PCT Filed: May 31, 2022
(86) PCT No.: PCT/US22/31658

§ 371 Date: Nov. 27, 2023

Related U.S. Application Data
(60) Provisional application No. 63/195,008, filed on May 29, 2021.

(51) Int. Cl. C12N 15/113

(52) U.S. Cl. CPC C12N 15/113; C12N 2310/14; C12N 2310/313; C12N 2310/321; C12N 2310/3341; C12N 2310/335; C12N 2310/533

(57) ABSTRACT

The present invention discloses a novel type of gene silencing technology for modulation of target nucleic acid and/or protein in cells, tissues, organisms and animals. The new technology provides compositions for use in gene targeting or gene silencing applications, including prevention and treatment of human diseases. The composition comprises an asymmetric, short, duplex DNA molecule where the sense strand is shorter than the antisense strand. The duplex DNA molecule further includes at least one interspersed segment of ribonucleotide monomer. The present invention further provides methods of using the compositions for modulating expression or function of a target gene, or for treatment or prevention of diseases as well as for other medical or biological applications.

### CROSS-REFERENCE TO RELATED APPLICATIONS

This application claims priority to and the benefit of co-pending U.S. provisional patent application Ser. No. 63/195,008, filed May 29, 2021, which application is incorporated herein by reference in its entirety.

### FIELD OF THE INVENTION

The invention relates to asymmetric short duplex DNA to be used as gene silencing technology as well as related compositions and methods that can be used in biological or medical research, in the treatment and prevention of diseases and for gene silencing applications in other biological fields.

### BACKGROUND OF THE INVENTION

Modern medical therapeutics is dependent on two fundamental technologies, namely small molecule chemistry and protein/antibody technology. However, only about 10% of the targets identified by genomic and biomedical research can be addressed by the two aforementioned cornerstone technologies. Oligonucleotides hold promise for addressing numerous targets, including non-druggable ones by small molecule chemistry and antibody/protein technologies. More than four decades of research have created Antisense oligonucleotide (ASO) and small interfering RNA (siRNA) technologies (Cy A. Stein et al., 2017). However, despite of over 40 years of research, other than a small number of clinical orphan indications, significant druggability issues have blocked the development of ASO and siRNA technologies from becoming a mainstay therapeutic platform. Such druggability issues include, among others, low silencing efficiency, off-target effects, stimulation of non-intended immune response, tissue penetration challenges, and in vivo delivery, etc. There is hence a significant unmet need to create novel technologies to target genes of interest in various biological and medical applications.

ASO is a gene silencing technology based upon a concept originally proposed in 1978 (Zamecnik P. C. et al., 1978). Generally, the principle behind the ASO technology is that an antisense oligonucleotide hybridizes to a target nucleic acid and modulates gene expression activities or function, such as transcription/post-transcription or translation. The mechanisms can be broadly categorized as: (1) occupancy only without promoting RNA degradation, in which the binding of the ASO leads to translational arrest, inhibition of splicing, or induction of alternatively spliced variants, or (2) occupancy-induced destabilization, in which the binding of the ASO promotes degradation of the RNA through endogenous enzymes, such as ribonuclease H1 (RNase H1); and (3) translation modulation: ASO can block upstream open reading frames (uORFs) or other inhibitory or regulatory elements in the 5′UTR, increasing or modulating translation efficiency (Stanley T. Crooke et al., 2008; C′. Frank Bennett, 2010; Richard G. Lee, 2013; Stanley T. (rooke, 2017). The ASO structure is a single-stranded deoxyribonucleotide sequence that can bind to target RNA through base-pairing. After 40 years of research, ASO technology has been improved through various chemical modifications of the single stranded oligonucleotide, such as phosphorothioate substitution or other modified nucleotides (See Iwamoto N et al 2017, Crooke S T, 2017; Crooke S T et al., 2018; U.S. Pat. Nos. 7,919,472 and 9,045,754).

RNAi is a mechanism by which short double-stranded RNA triggers the loss of RNA of homologous sequence, and was first observed in plants and demonstrated in nematodes (Caenorhabditis elegans) (A. Fire et al, 1998). The mechanism involved degradation of a long dsRNA into short interfering duplex RNAs (siRNA), and interaction of the siRNA with a multi-protein RNA-Induced Silencing Complex (RISC); within the RISC, the siRNA is unwound, the sense strand is discarded, and the antisense or guide strand binds to the RISC endonuclease AGO2, which then cleaves the target RNA (de Fougerolles et al., 2007; Ryszard Kole, 2016). In mammalian cells, synthetic siRNA or asymmetric short interfering RNAs (aiRNA or asymmetric siRNA) can be used to induce gene silencing through RISC-dependent mechanism (See Elbashir S M et al., 2001; Sun X et al., 2008; U.S. Pat. Nos. 7,056,704 and 9,328,345).

Oligonucleotides have been studied for decades and are considered to hold significant promise for becoming a whole new class of therapeutics. However, limited silencing efficiency, delivery challenges and dose-dependent adverse effects, including hybridization-dependent toxicities and hybridization-independent toxicities, of oligonucleotides continue to limit the development of these novel classes of therapeutics (C. Frank Bennett, 2010; C. Frank Bennett, 2019; Roberts T O et al., 2020; Crooke S T et al., 2018; and Setten R L et al., 2020). In general, ASO compounds are less potent than siRNA-based compounds in inducing gene silencing, yet ASO compounds have some pharmaceutical advantages than siRNA compounds. Currently ASO and siRNAs remain the two equally important platform technologies for designing gene-silencing therapeutics (Crooke S T et al 2018; Roberts T O et al 2020). The Hybridization-dependent toxicities of oligonucleotides are mainly attributed to hybridization to non-target genes (“off-target effects”) (Jackson et al., 2003; Lin X et al., 2005). Hybridization-independent toxicities happened through the interactions of the oligonucleotide with proteins: the effects include increased coagulation time, pro-inflammatory effects and activation of the complement pathway. These effects tend to occur at higher doses of oligonucleotides and are dose-dependent. For example, at higher concentrations, ASOs lead to renal tubule changes and thrombocytopenia (Geary, R S. et al., 2007; Kwoh J T, 2008). Clinically, the primary tolerability and safety issues for first-generation PS antisense oligodeoxynucleotides and second-generation 2′-MOE-modified antisense oligonucleotides have proven to be hybridization-independent effects such as prolongation of activated partial thromboplastin time, injection site reaction, and constitutional symptoms such as fever, chills, and headache (C. Frank Bennett, 2010; Henry S P, 2008; Kwoh J T, 2008). Even the most optimized ASOs generally are still far less potent than siRNA and have proven to have dosage-dependent stereotypic toxicity (Kendall S. Frazier, 2015). In order to alleviate oligonucleotides dose-dependent toxicities, efforts have been made in the past 40 years to overcome limited efficacy issues and associated safety problems through various chemical modifications (Iwamoto N et al 2017, Crooke S T et al., 2018; and Roberts T O et al., 2020).

Compared to ASOs, off-target effects of siRNA duplex are considered to be mediated by sense strand-mediated silencing, competition with endogenous miRNA pathways and interaction with TLR or other proteins (Setten R L. et al 2019). In addition, the typical siRNA duplex of 21 nt/19 bp is not efficient in cell and tissue penetration, also requires extensive chemical modifications to enhance stability and other pharmaceutical properties. Asymmetric siRNA (or aiRNA) was designed to overcome off-target effects mediated by sense strand of the symmetric siRNA as well as other off-target mechanism (See Sun X et al., 2008; Grimm D), 2009; Selbly (R et al., 2010; and PCT Patent publication WO2009029688).

In summary, after more than 40 years of innovation in ASO technology and more than 20 years of research in RNAi-based technologies, successful development of gene-targeted therapies against nearly 90% of targets implicated in human diseases remain challenging. Moreover, the current approved oligonucleotide drugs cost more than half a million USD per patient/per year, making it impossible to address diseases affecting the general populations. As such, novel technologies to overcome these challenges are urgently needed.

The references cited herein are not admitted to be prior art to the claimed invention.

### SUMMARY OF THE INVENTION

The present invention is based on a surprising discovery of potent gene silencing triggered by asymmetric short duplex deoxyribonucleotides (asdDNA, asymmetric sdDNA). This novel type of gene silencing technology enabled by asdDNA with one or more interspersed ribonucleotides employs a short, duplex molecule made up by linked nucleotide monomers that are each selected from the group of naturally occurring nucleotide, analogs thereof, and modified nucleotide (hereinafter, collectively referred to as “nucleotide monomers”). In other words, nucleotide monomers used in an embodiment of the present invention include “deoxyribonucleotide monomers” selected from the group of naturally occurring deoxyribonucleotides, analogs thereof, and modified deoxyribonucleotides. Furthermore, the gene silencing function of asdDNA can be dramatically enabled or enhanced by incorporating one or a few interspersed ribonucleotide monomers. The “ribonucleotide monomers” can be selected from the group of naturally occurring ribonucleotides, analogs thereof, and modified ribonucleotides.

In the present invention, a short duplex DNA (sdDNA) molecule, or even more specifically, an asymmetric short duplex DNA (asdDNA) molecule is further interspersed with ribonucleotide monomers, which form at least one interspersed segment of ribonucleotide monomer(s) (“ISR”).

The great gene silencing effect of the asdDNA-based novel platform technology contained in the present disclosure is, in one embodiment, achieved through a sense strand of oligonucleotide monomers and an antisense strand of oligonucleotide monomers that is substantially complementary to a targeted ribonucleotide sequence. Our data have shown that asdDNA molecules of the present invention, with their unique and novel compositions, can trigger gene silencing at pico molar concentrations, which are more potent than existing antisense technology and siRNA technology, and therefore enabling reduction of dose-dependent toxicities. The asdDNA molecules of the present invention are also expected to have at least one of the following advantages over existing gene silencing technologies including better tissue penetration; enabling gene silencing in in nucleus, in mitochondria etc., in contrast siRNA-based gene silencing only occurs in cytoplasm; reduced off-target effects; better stability; elimination or reduction of undesired competition with endogenous microRNA pathways associated with siRNAs; lower synthesis cost and other improved pharmaceutical properties. Therefore, the asdDNA molecules of the present invention have great potential for addressing a variety of challenges facing ASO, siRNA and other existing gene silencing technologies. The asdDNA molecules of the present invention can be used in all areas that current oligonucleotides are being applied or contemplated for use, including research, diagnosis, disease prevention and therapies as well as other applications in biological fields, including agriculture and veterinary medicine.

In a first aspect, the present invention provides a composition comprising a short duplex DNA (sdDNA) molecule that has a first and longer strand than the second strand. In other words, the sdDNA molecule is an asymmetric short duplex DNA (asdDNA) molecule where the second strand is shorter than the first strand. The first strand is substantially complementary to a targeted segment of a targeted RNA through at least one targeting region, and can therefore be considered an antisense strand or an antisense oligonucleotide. Further, the second strand, which can be considered a sense strand or a sense oligonucleotide, is substantially complementary to the first strand, and forms at least one double-stranded region with the first strand. The asdDNA molecule includes at least one interspersed segment of ribonucleotide monomer(s) (ISR). In a feature, the ISR in the asdDNA molecule includes at least one ribonucleotide monomer(s), which can be in either or both strands.

The composition provided by the present invention is used for modulating gene expression or function in a eukaryotic cell, wherein the asdDNA is caused to contact a cell or administered to a subject.

In some embodiments, the asdDNA molecule includes at least one or at least two interspersed segment of ribonucleotide monomer(s) (ISR). In a feature of the asdDNA molecule of the invention, the first strand of the molecule includes at least one ISR. In an embodiment, the first strand includes at least one ISR and the second strand also includes at least one ISR. In a feature, each ISR, independently of each other, either consists of one ribonucleotide monomer, or comprises at least 2, 3, 4 or 5 contiguous ribonucleotide monomers. In another feature, the ISR includes at least 2 ribonucleotide monomers, whether they are contiguous or spaced apart with at least one (1, 2, 3, 4, 5, 6, 7, 8, 9, 10 or more) intervening monomer of a different kind. In yet another feature, the total number of ribonucleotide monomers of all ISR(s) in the first strand is at least 2.

In one feature, at least one ISR is disposed in at least one targeting region of the first (antisense) strand. In another feature, at least one ISR is disposed in at least one double-stranded region of the second (sense) strand. In yet another feature, at least one ISR is disposed in at least one targeting region of the first strand and at least one ISR is disposed in at least one double-stranded region of the second (sense) strand. In some embodiments, at least one ISR can be deposited at any position of the first strand. In some embodiments, at least one ISR is positioned at or near (within 7 nucleobases or within 33% of the total number of nucleobases in the said strand from a terminal counting said terminal, e.g., starting from the terminal, position no. 1, 2, 3, 4, 5, 6, or 7 for a strand that is about 21 nucleobases long) the 5′ end of the first strand; and/or at or near (within 7 nucleobases or within 33% of the total number of nucleobases in the said strand from a terminal counting said terminal) the 3′ end of the first strand; and/or at a more central part of the first strand. In some embodiments, at least one ISR disposed in the first strand is positioned at only overhang region of the first strand. In some embodiments, at least one ISR disposed in the first strand is positioned at both overhang region and double-stranded region of the first strand. In some embodiments, ISR(s) in the first strand comprise at least one ribonucleotide monomer positioned at the 5′ end of the first strand or the 3′ end of the first strand. In some embodiments, at least one ISR is positioned at or near (within 7 nucleobases or within 33% of the total number of nucleobases in the said strand from a terminal counting said terminal) the 5′ end of the second strand; and/or at or near (within 7 nucleobases or within 33% of the total number of nucleobases in the said strand from a terminal counting said terminal) the 3′ end of the second strand; and/or at a more central part of the second strand.

In one feature, the first strand or the antisense strand includes multiple linked nucleotide monomers forming a nucleobase sequence, and is at least 70%, 80%, 85%, 90%, 95% complementary or fully complementary to the targeted segment of the targeted gene's RNA. In certain embodiments, the targeted RNA is selected from mRNA or non-coding RNA where the RNA either encodes a protein or regulates a part of a biological pathway implicated in a disease, e.g., a mammalian disease. The terms “target” and “targeted” are used interchangeably in the present disclosure and share the same meaning.

In various embodiments, the first/antisense strand has a backbone length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 linked nucleotide monomers, or equivalents thereof, or of a range bracketed by any two of the above values (both range endpoints included). For example, some of the ranges of the length of the first antisense strand include: (a) 8-33 nucleotide monomers; (b) 10-30 nucleotide monomers; (c) 10-29 nucleotide monomers; (d) 12-29 nucleotide monomers; (e) 12-28 nucleotide monomers; (f) 12-26 nucleotide monomers; (g) 12-25 nucleotide monomers; (h) 13-25 nucleotide monomers; (i) 13-24 nucleotide monomers; (j) 13-23 nucleotide monomers; (k) 15-23 nucleotide monomers; (1) 8-50 nucleotide monomers; (m) 10-36 nucleotide monomers; (n) 12-36 nucleotide monomers; (o) 12-32 nucleotide monomers; (p) 14-36 nucleotide monomers; and (q) at least 8 nucleotide monomers.

In one feature, the second strand or the sense strand includes multiple linked nucleotide monomers forming a nucleobase sequence, and is at least 70%, 75%, 80%, 85%, 90%, 95% complementary or fully complementary to at least one linked region of the first strand or the antisense strand. In some embodiments, the sense strand is fully complementary to at least one linked region of the first/antisense strand, and forms the at least one double-stranded region without any mismatch. In some embodiments, the sense strand is complementary to at least one linked region of the first/antisense strand, and forms the at least one double-stranded region with 1, 2, 3 or more mismatches. In one feature, the mismatched monomer(s) in the sense strand has a nucleobase selected from the group consisting of A, G, C, and T or a modified nucleobase. In some embodiments, at least one of the first base and the last base of the second strand is complementary to base in the first strand. In some embodiments, at least the first base and the last base of the second strand are complementary to a nucleobase in the first strand.

In one feature, the second strand or the sense strand has a backbone length shorter than the first strand or the antisense strand by at least a number of nucleotide monomers as follows: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 and 38. In various embodiments, the second or sense strand has a backbone length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or 36 linked nucleotide monomers, or equivalents thereof, or of a range bracketed by any two of the above values (both range endpoints included). In certain embodiments, for example, some of the ranges of the length of the second sense strand include: (a) 8-32 nucleotide monomers; (b) 8-30 nucleotide monomers; (c) 8-29 nucleotide monomers; (d) 9-29 nucleotide monomers; (e) 9-26 nucleotide monomers; (f) 9-25 nucleotide monomers; (g) 10-29 nucleotide monomers; (h) 10-28 nucleotide monomers; (i) 10-26 nucleotide monomers; (j) 10-25 nucleotide monomers; (k) 11-24 nucleotide monomers; (1) 12-23 nucleotide monomers; (m) 12-23 nucleotide monomers; (n) 12-22 nucleotide monomers tides; (o) 13-23 nucleotide monomers; (p) 15-23 nucleotide monomers tides; (q) 8-35 nucleotide monomers tides; (r) 8-33 nucleotide monomers tides; (s) 9-35 nucleotide monomers tides; (t) 9-34 nucleotide monomers tides; (u) 9-32 nucleotide monomers tides; (v) 9-30 nucleotide monomers tides; (w) 10-30 nucleotide monomers tides; (x) 10-32 nucleotide monomers tides; (y) at least 8 nucleotide monomers and (z) at least 6 nucleotide monomers. In certain embodiments, the second strand can have a backbone length of any number of nucleotide monomers that is fewer than that of the first strand, provided that a duplex can be formed with the first strand thermodynamically.

In a feature, the two ends of the first strand are one of the following configurations: a 3′-overhang and a 5′-overhang; a 3′-overhang and a blunt end at 5′ end; a 5′-overhang and a blunt end at 3′ end; 3′-overhang and 5′ recessed-end; or 5′-overhang and 3′ recessed-end. In certain embodiments, the 3′-overhang of the first strand has a length of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide monomers, or of a range bracketed by any two of the above values (both range endpoints included). In various embodiments, the 3′-overhang of the first strand has a length of 1-15, 1-10, 1-8, or 1-5 nucleotide monomers (both range endpoints included).

In certain embodiments, the 5′-overhang of the first strand has a length of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide monomers, or of a range bracketed by any two of the above values (both range endpoints included). In various embodiments, the 5′-overhang of the first strand has a length of 1-15, 1-10, 1-8, or 1-5 nucleotide monomers (both range endpoints included).

In an embodiment of the invention, the first strand has a 3′-overhang of 1-15 nucleotide monomers and a 5′-overhang of 1-15 nucleotide monomers. In another embodiment, the first strand has a 3′-overhang of 1-26 nucleotide monomers and a 5′ blunt end or a 5′ recessed end. In yet another embodiment, the first strand has a 5′-overhang of 1-26 nucleotide monomers and a 3′ blunt end or a 3′ recessed end.

In a feature, the two ends of the second strand are one of the following configurations: a 3′-overhang and a 5′ recessed-end; a 5′-overhang and a 3′ recessed-end; 3′-blunt-end and 5′ recessed-end; 5′ blunt-end and 3′ recessed-end; 3′ recessed-end and a 5′ recessed end. In certain embodiments, the 3′-overhang of the second strand has a length of 1, 2, 3, 4, 5, 6, 7, 8, 9, 10, 11, 12, 13, 14, 15, 16, 17, 18, 19, 20, 21, 22, 23, 24, or 25 nucleotide monomers. In various embodiments, the 3′-overhang of the second strand has a length of 1-15, 1-10, 1-8, or 1-5 nucleotide monomers (both range endpoints included). In certain embodiments, the 5′-overhang of the second strand has a length of 1, 2, 3, 4, 5, 6, 7, 8, 9, 10, 11, 12, 13, 14, 15, 16, 17, 18, 19, 20, 21, 22, 23, 24, or 25 nucleotide monomers. In various embodiments, the 5′-overhang of the second strand has a length of 1-15, 1-10, 1-8, or 1-5 nucleotide monomers (both range endpoints included).

In a feature of the asdDNA molecule of the invention, at least one nucleotide monomer in the first strand and/or the second strand is a modified nucleotide or nucleotide analogue, e.g., a sugar-, backbone-, and/or base-modified nucleotide. In an embodiment, such a backbone-modified nucleotide has at least a modification in an internucleoside linkage, e.g., to include at least one of a nitrogen or sulphur heteroatom. In some embodiments, the modified internucleoside linkage is or includes: phosphorothioate (P═S) group, phosphotriesters, methylphosphonates, or phosphoramidate.

In certain embodiments, the first strand and/or the second strand comprises at least one modified internucleoside linkage, where the modified internucleoside linkage is a phosphorothioate internucleoside linkage. In some embodiments, each internucleoside linkage of the first strand and/or the second strand is a phosphorothioate internucleoside linkage. In various embodiments, the internucleoside linkages of the first strand and/or the second strand are a mixture of phosphorothioate and phosphodiester linkages.

In a feature, the first strand and/or the second strand of the molecule of the invention comprises at least one modified nucleotide or nucleotide analogue that includes a modified sugar moiety. In certain embodiments, the 2′ position of the modified sugar moiety is replaced by a group selected from OR, R, halo, SH, SR, NH₂, NHR, NR₂, or CN, where each R is independently C₁-C₆ alkyl, alkenyl or alkynyl, and halo is F, Cl, Br or I. In some embodiments, the 2′ position of the modified sugar moiety is replaced by a group selected from allyl, amino, azido, thio, O-allyl, O-C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), or O—CH₂—C(—O)—N(Rₗ)—(CH₂)₂—N(Rₘ)(Rₙ), where each Rₗ, Rₘ and Rₙ is, independently, H or substituted or unsubstituted C₁-C₁₀ alkyl. In some embodiments, the modified sugar moiety has substituent group(s) selected from the group of 5′-vinyl, 5′-methyl (R or S), 4′-S, 2′-F, 2′-OCH₃2′-OCH₂CH₃, 2′-OCH₂CH₂F, 2′-O-aminopropylation (2′-AP) and 2′-O(CH₂)₂OCH₃. In some embodiments, the modified sugar moiety is substituted by a bicyclic sugar selected from the group of 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, 4′-C(CH₃)(CH₃) 0-2′, 4′-CH₂—N(OCH₃)-2′, 4′-CH₂—O—N(CH₃)-2′, 4′-CH₂—N(R)—O-2′ (where R is H, C₁-C₁₂ alkyl, or a protecting group), 4′-CH₂—C(H)(CH₃)-2′, and 4′-CH₂—C—(—CH₂)-2′. In some embodiments, the modified sugar moiety is selected from the group of 2′-O-methoxyethyl modified sugar (MOE), a 4′-(CH₂)—O-2′ bicyclic sugar (LNA), 2′-deoxy-2′-fluoroarabinose (a 2′-F-arabino, FANA), and a methyl(methyleneoxy) (4′-CH(CH₃)—O-2) bicyclic sugar (cEt).

In a feature of the asdDNA molecule of the invention, the sugar moiety of the deoxyribonucleotide monomer is either the sugar moiety of a naturally occurring deoxyribonucleotide (2-H) or 2′-deoxy-2′-fluoroarabinose (FA).

In a feature of the asdDNA molecule of the invention, the sugar moiety of the ribonucleotide monomer is selected from a naturally occurring ribonucleotide (2-OH), 2′-F modified sugar, 2′-OMe modified sugar, 2′-O-methoxyethyl modified sugar (MOE), a 4′-(CH₂)—O-2′ bicyclic sugar (LNA) and a methyl(methyleneoxy) (4′-CH(CH₃)—O-2) bicyclic sugar (cEt).

In another feature, the first strand and/or the second strand of the molecule of the invention includes at least one nucleotide monomer comprising a modified nucleobase. In some embodiments, the modified nucleobase is selected from the group of: 5-methylcytosine (5-Me-C), inosine base, a tritylated base, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 1-methyl-pseudo-uracil, 8-halo, 8-amino, 8-thiol, 8-thioalkyl, 8-hydroxyl and other 8-substituted adenines and guanines, 5-halo particularly 5-bromo, 5-trifluoromethyl, 5-methyl uridine and other 5-substituted uracils and cytosines, 7-methylguanine and 7-methyladenine, 2-F-adenine, 2-aminoadenine, 8-azaguanine and 8-azaadenine, and 7-deazaguanine and 7-deazaadenine and 3-deazaguanine and 3-deazaadenine. In a particular embodiment, the modified nucleobase is a 5-methylcytosine. In an embodiment, each cytosine base in the molecule of the invention is 5-methylcytosine. In an embodiment, each uridine base in the ISR of the asdDNA molecule of the invention is 5-methyluridine.

In a feature, the asdDNA molecule of the present invention may include at least one CpG motif that can be recognized by the pattern recognition receptors (PRR), e.g., Toll-like receptors.

In one feature, the first strand and/or the second strand of the molecule of the invention is conjugated to a ligand or a moiety. In certain embodiments, the ligand or moiety is selected from the group of: peptide, antibody, polymer, polysaccharide, lipid, hydrophobic moiety or molecule, cationic moiety or molecule, lipophilic compound or moiety oligonucleotide, cholesterol, GalNAc and aptamer.

In a feature of the invention, the asdDNA molecule is used for modulating gene expression or function in a cell, e.g., a eukaryotic cell such as a mammalian cell.

In certain embodiments, the targeted RNA, which dictates at least part of the nucleotide monomer sequence of the asdDNA molecule according to principles of the invention, is selected from mRNA or non-coding RNA wherein such RNA either encodes a protein or regulates a part of a biological pathway implicated in a disease. Such target RNA, in various embodiments, can be selected from: an mRNA of a gene implicated in human or animal diseases or condition; an mRNA of a gene of a pathogenic microorganism; a viral RNA, and a RNA implicated in a disease or disorder selected from the group consisting of autoimmune diseases, inflammatory diseases, degenerative diseases, infectious diseases, proliferative diseases, metabolic diseases, immune-mediated disorders, allergic diseases, dermatological diseases, malignant diseases, gastrointestinal disorders, respiratory disorders, cardiovascular disorders, renal disorders, rheumatoid disorders, neurological disorders, endocrine disorders, and aging-related disorders or diseases.

In an embodiment, the invention provides an asymmetric short duplex DNA (asdDNA) molecule comprising a first strand and a second strand each comprising linked nucleotide monomers selected from the group of nucleotides, analogs thereof, and modified nucleotides, where: (a) the first strand is longer than the second strand by at least a number of monomers selected from the group of 1, 2, 3, 4, 5, 6, 7, 8, 9 and 10 monomers; (b) the first strand is substantially complementary to a targeted segment of a targeted RNA through at least one targeting region, and wherein the first strand consists of 10-36 (both range endpoints included) nucleoside monomers linked through a linkage selected from the group consisting of a phosphorothioate linkage, a phosphodiester linkage, and a mixture of phosphorothioate and phosphodiester linkages between adjacent monomers; (c) the second strand is substantially complementary to the first strand, and forms at least one double-stranded region with the first strand, and wherein the second strand consists of 8-32 (both range endpoints included) nucleoside monomers linked through a linkage selected from the group consisting of a phosphorothioate linkage, a phosphodiester linkage, and a mixture of phosphorothioate and phosphodiester linkages between adjacent monomers; (d) the asdDNA molecule comprises at least one interspersed segment of ribonucleotide monomers (ISR) linked to at least one deoxyribonucleotide monomer selected from the group consisting of a deoxyribonucleotide, an analog thereof, and a modified deoxyribonucleotide; (e) the ISR in the asdDNA molecule comprises at least one ribonucleotide monomer selected from the group consisting of a ribonucleotide, an analog thereof, and a modified ribonucleotide. In a feature, the asdDNA molecule is used for modulating a target gene expression or function in a cell, e.g., a eukaryotic cell such as a mammalian cell. In a further feature, the asdDNA molecule is more potent or more efficacious at silencing the expression of the target gene than a corresponding ASO in a cell.

In a second aspect, the present invention provides a pharmaceutical composition comprising the composition in the first aspect as active agent, and a pharmaceutically acceptable excipient, carrier, or diluent. Examples of such carriers include and are not limited to: a pharmaceutical carrier, a positive-charge carrier, a liposome, a lipid nanoparticle, a protein carrier, a hydrophobic moiety or molecule, a cationic moiety or molecule, GalNAc, a polysaccharide a polymer, a nanoparticle, a nanoemulsion, a cholesterol, a lipid, a lipophilic compound or moiety, and a lipoid.

In a third aspect, the present invention provides a method of using the composition in the first aspect or the pharmaceutical composition in the second aspect for treating or preventing a disease or a condition by administering a therapeutically effective amount of an asdDNA molecule of the invention or a pharmaceutical composition containing such a molecule. The administration method is a route selected from the group of intravenous injection (iv), subcutaneous injection (sc), per os (po), intramuscular (im) injection, oral administration, inhalation, topical, intrathecal, and other regional administrations.

In a feature, the disease or condition being prophylactically or therapeutically treated is selected from the group of cancer, autoimmune disease, inflammatory diseases, degenerative diseases, infectious diseases, proliferative diseases, metabolic diseases, immune-mediated disorders, allergic diseases, dermatological diseases, malignant diseases, gastrointestinal disorders, hepatic disorders, respiratory disorders, cardiovascular disorders, dermatological disorders, renal disorders, rheumatoid disorders, neurological disorders, psychiatric disorders, endocrine disorders, and aging-related disorders or diseases.

In a fourth aspect, the present invention provides a method of using the composition in the first aspect or the pharmaceutical composition in the second aspect for regulating or modulating a gene expression or gene function in a eukaryotic cell. The method comprises the step of contacting the cell with an effective amount of any asdDNA molecule of the invention or a pharmaceutical composition containing such a molecule.

In one embodiment, said contacting step comprises the step of introducing a composition comprising said asdDNA molecule into a target cell in culture or in an organism in which the selective gene silencing can occur. In a further embodiment, the introducing step is selected from the group consisting of simple mixing, transfection, lipofection, electroporation, infection, injection, oral administration, intravenous injection (iv), subcutaneous injection (sc), per os (po), intramuscular (im) injection, inhalation, topical, intrathecal, and other regional administrations. In another embodiment, the introducing step comprises using a pharmaceutically acceptable excipient, carrier, or diluent selected from the group that includes a pharmaceutical carrier, a positive-charge carrier, a lipid nanoparticle, a liposome, a protein carrier, a hydrophobic moiety or molecule, a cationic moiety or molecule, GalNAc, a polysaccharide a polymer, a nanoparticle, a nanoemulsion, a cholesterol, a lipid, a lipophilic compound or moiety, and a lipoid.

In certain embodiments, the target gene is mRNA. In certain embodiments, the target gene is non-coding RNA, such as microRNA and lncRNA.

In an embodiment, the target gene is associated with a disease, a pathological condition, or an undesirable condition in a mammal. In a further embodiment, the target gene is a gene of a pathogenic microorganism. In an even further embodiment, the target gene is a viral gene. In another embodiment, the target gene is a tumor-associated gene. In yet another embodiment, the target gene is a gene associated with a disease selected from the group listed with respect to the third aspect.

In another aspect, the invention provides an asymmetric oligomeric duplex comprising (a) one or more deoxyribonucleosides, analogs thereof or modified deoxyribonucleosides, and (b) one or more ISR comprising ribonucleosides, analogs thereof or modified ribonucleosides, linked into an antisense sequence of at least 8 nucleobases in length. The antisense sequence is at least 70% complementary to a target sequence.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention. While several embodiments have been shown and described, any modifications may be made without departing from the spirit and scope of the present invention.

### BRIEF DESCRIPTION OF FIGURES

FIG. 1 shows representative target genes, and representative target sequences used in examples, as well as exemplary sequences of corresponding antisense strand of a molecule that can be used for silencing the target gene.

FIG. 2A illustrate exemplary structures of some embodiments of asymmetric short duplex DNAs (asdDNAs) with at least one interspersed segment of ribonucleotide monomers (ISR) in antisense strand (first strand) and/or sense strand (second strand). In each duplex depicted here, the sense strand is listed on top of the antisense strand. FIG. 2B shows exemplary sequences of asdDNAs having structure in FIG. 2A for targeting the APOCIII gene.

FIG. 2C shows the gene silencing potency of asdDNAs (FIG. 2B) targeting APOCIII. Relative mRNA levels of the APOCIII gene were detected after transfecting the asdDNAs at 100 pM in HepaRG cells.

FIG. 3A illustrates exemplary structures of some embodiments of asdDNAs with at least one ISR in the antisense strand. FIG. 3B shows exemplary sequences of asdDNAs having structure in FIG. 3A for targeting the APOCIII gene. FIG. 3C shows the gene silencing potency of asdDNAs (FIG. 3B) targeting the APOCIII gene. Relative mRNA levels of the APOCIII gene were detected after transfecting the asdDNAs at 100 pM in HepaRG cells.

FIG. 4A illustrates exemplary structures of some embodiments of asdDNAs with non-modified DNA sense strand and at least one ISR in the antisense strand. FIG. 4B shows exemplary sequences of asdDNAs in FIG. 4A for targeting the APOCIII gene. FIG. 4C shows the gene silencing potency of asdDNAs (FIG. 4B) targeting the APOCIII gene. Relative mRNA levels of the APOCIII gene were detected after transfecting the asdDNAs at 100 pM in HepaRG cells.

FIG. 5A illustrates exemplary structures of asdDNAs with various motif of ISR in antisense strand and exemplary sequences of the asdDNAs for targeting the APOCIII gene. The various motif of ISR in the antisense strand in FIG. 5A have various number of ribonucleotide monomers and positions of the ISR(s) in the antisense strand. FIG. 5B shows the gene silencing potency of asdDNAs targeting the APOCIII gene shown in FIG. 5A and comparison with the gene silencing potency of corresponding ASO (each corresponding ASO has the same sequence as the antisense strand of each asdDNAs in FIG. 5A). Relative mRNA levels of the APOCIII gene were detected after transfecting the asdDNAs and corresponding ASO at 100 pM in HepaRG cells.

FIG. 6A illustrates exemplary structures of some embodiments of asdDNAs with various positions of ISR in the antisense strand and exemplary sequences of the asdDNAs for targeting the APOCIII gene. FIG. 6B shows the gene silencing potency of asdDNAs targeting the APOCIII gene shown in FIG. 6A and comparison with the gene silencing potency of corresponding ASO (each corresponding ASO has the identical sequence as the antisense strand of each asdDNAs in FIG. 6A). Relative mRNA levels of the APOCIII gene were detected after transfecting the asdDNAs and corresponding ASO at 100 pM in HepaRG cells.

FIG. 7A shows exemplary structures of some embodiments of asdDNAs with different lengths of antisense strand and exemplary sequences of the asdDNAs for targeting the APOCIII gene. FIG. 7B shows the gene silencing potency of asdDNAs targeting the APOCIII gene shown in FIG. 7A and comparison with the gene silencing potency of corresponding ASO (each corresponding ASO has the identical sequence as the antisense strand of each asdDNAs in FIG. 7A). Relative mRNA levels of the APOCIII gene were detected after transfecting the asdDNAs and corresponding ASO at 100 pM in HepaRG cells.

FIG. 8A shows exemplary structure and sequence of some embodiments of asdDNAs with various lengths motif of antisense strand and sense strand. FIG. 8B shows the gene silencing potency for targeting the APOCIII gene of asdDNAs shown in FIG. 8A. FIG. 8C shows the gene silencing potency for targeting the APOCIII gene of corresponding ASO has the identical sequence as the antisense strand of each asdDNAs shown in FIG. 8A. The gene silencing potency of relative mRNA levels of the APOCIII gene were detected after transfecting the asdDNAs and corresponding ASO at 100 pM in HepaRG cells.

FIG. 9A shows exemplary structure and sequence of some embodiments of asdDNAs with various lengths motif of antisense strand and sense strand. FIG. 9B shows the gene silencing potency for targeting the APOCIII gene of asdDNAs shown in FIG. 9A. The relative mRNA levels of the APOCIII gene were detected after transfecting the asdDNAs at 100 pM in HepaRG cells.

FIG. 10A shows exemplary structure and sequence of some embodiments of asdDNAs with various lengths of sense strand and a fixed length of antisense strand. FIG. 10B shows the gene silencing potency for targeting the APOCIII gene of asdDNAs shown in FIG. 10A. The relative mRNA levels of the APOCIII gene were detected after transfecting the asdDNAs at 100 pM in HepaRG cells.

FIG. 11A also shows exemplary structure and sequence of some other embodiments of asdDNAs with various lengths of sense strand and a fixed length of antisense strand. FIG. 11B shows the gene silencing potency for targeting the APOCIII gene of asdDNAs shown in FIG. 11A. The relative mRNA levels of the APOCIII gene were detected after transfecting the asdDNAs at 100 pM in HepaRG cells.

FIG. 12A also shows exemplary structure and sequence of some other embodiments of asdDNAs with various lengths of antisense strand and a fixed length of sense strand. FIG. 12B shows the gene silencing potency for targeting the APOCIII gene of asdDNAs shown in FIG. 12A. The relative mRNA levels of the APOCIII gene were detected after transfecting the asdDNAs at 100 pM in HepaRG cells.

FIG. 13A shows exemplary structure and sequence of some embodiments of asdDNAs with various motif of ISR in antisense strand. FIG. 13B shows the gene silencing potency for targeting the APOCIII gene of asdDNAs shown in FIG. 13A. The relative mRNA levels of the APOCIII gene were detected after transfecting the asdDNAs at 100 pM in HepaRG cells.

FIG. 14A shows exemplary structure and sequence of some embodiments of asdDNAs with at least one mismatch in the antisense strand when hybridize to target gene. FIG. 14B shows the gene silencing potency for targeting the APOCIII gene of asdDNAs shown in FIG. 14A. The relative mRNA levels of the APOCIII gene were detected after transfecting the asdDNAs at 100 pM in HepaRG cells.

FIG. 15A shows exemplary structure and sequence of some embodiments of asdDNAs with at least one mismatch in the sense strand when form double-stranded region with the antisense strand. FIG. 15B shows the gene silencing potency for targeting the APOCIII gene of asdDNAs shown in FIG. 15A. The relative mRNA levels of the APOCIII gene were detected after transfecting the asdDNAs at 100 pM in HepaRG cells.

FIG. 16A shows the structure and sequence of an exemplary asdDNA of this invention and its corresponding siRNA for targeting the STAT3 gene as well as gene silencing potency comparison between the asdDNA and siRNA as determined by IC50 and IC90. FIG. 16B illustrates the comparison of gene silencing potency by asdDNA and by siRNA shown in FIG. 16A at 100 pM, 1 nM and 10 nM in HepaRG cells, respectively.

FIG. 17 shows structures, sequences and gene silencing potency of exemplary asdDNAs targeting the APOCIII gene in comparison with corresponding ASOs.

FIG. 18 shows structures, sequences and gene silencing potency of exemplary asdDNAs targeting the APOB gene in comparison with corresponding ASO.

FIG. 19 shows structures, sequences and gene silencing potency of exemplary asdDNAs targeting the TTR gene in comparison with corresponding ASO.

FIG. 20 shows structures, sequences and gene silencing potency of exemplary asdDNAs targeting the STAT3 gene.

FIG. 21 shows structures, sequences and gene silencing potency of exemplary asdDNA targeting the β-Catenin gene at 100 pM, 200 pM, 1 nM, 3 nM, 10 nM and 30 nM in DLD1 cells, respectively.

### DETAILED DESCRIPTION OF THE INVENTION

The present invention refers to gene or RNA modulation/silencing technology using short duplex DNAs. This new technology is used for modulation of gene expression or function in vitro and in vivo by using an asymmetric short duplex DNA (asdDNA) composition. The present invention also provides methods of using the compositions for modulating expression or function of a target gene, or for treatment or prevention of diseases as well as for other medical and biological applications. These composition and methods provide high potency in regulating gene expression or gene function, but also reduces dose-dependent toxicities.

### 1. DEFINITIONS

As used herein, the singular form “a”, “an”, and “the” include plural references unless the context clearly dictates otherwise. For example, the term “a cell” includes a plurality of cells including mixtures thereof.

When the term “about” is used in conjunction with a numerical range, it modifies that range by extending the boundaries above and below those numerical values. In general, the term “about” is used herein to modify a numerical value above and below the stated value by a variance of 20%, 10%, 5%, or 1%. In some embodiments, the term “about” is used to modify a numerical value above and below the stated value by a variance of 10%. In some embodiments, the term “about” is used to modify a numerical value above and below the stated value by a variance of 5%. In some embodiments, the term “about” is used to modify a numerical value above and below the stated value by a variance of 1%.

As used herein, the term “analog” or “analogue,” interchangeably, means a functional or structural equivalent. For instance, nucleoside and nucleotide analogues have been used in clinical treatment of cancer and viral infections for decades and new compounds are continually synthesized and evaluated by the researchers and the pharmaceutical industry, see, e.g., Jordheim L. P. et al., Nat Rev Drug Discov 12, 447-464 (2013).

As used herein, the term “deoxyribonucleoside monomer” means a nucleoside monomer that includes a naturally occurring deoxyribonucleoside, an analog thereof, and a modified deoxyribonucleoside. The term “deoxyribonucleotide monomer” means a nucleotide monomer that includes a naturally occurring deoxyribonucleotide, an analog thereof, and a modified deoxyribonucleotide.

As used herein, the term “ribonucleoside monomer” means a nucleoside monomer that includes a naturally occurring ribonucleoside, an analog thereof, and a modified ribonucleoside. The term “ribonucleotide monomer” means a nucleotide monomer that includes a naturally occurring ribonucleotide, an analog thereof, and a modified ribonucleotide.

As used herein, the term “nucleoside” means a compound comprising a nucleobase moiety and a sugar moiety. Nucleoside monomers include, but are not limited to, naturally occurring nucleosides (e.g., deoxyribonucleosides and ribonucleosides as found in DNA and RNA, respectively), analogs thereof and modified nucleosides. A nucleoside monomer can be either a deoxyribonucleoside monomer or a ribonucleoside monomer. Nucleoside monomers may be linked to a phosphate moiety to become, for example, nucleotide monomers.

As used herein, the term “nucleotide” means a nucleoside further comprising a phosphate linking group. Nucleotide monomers include, but are not limited to, naturally occurring nucleotides (e.g., deoxyribonucleotides and ribonucleotides as found in DNA and RNA, respectively), analogs thereof and modified nucleotides. A nucleotide monomer can be either a deoxyribonucleotide monomer or a ribonucleotide monomer. A modified nucleotide may be modified at one of more of the following: its nitrogen-containing nucleobase moiety, its five-carbon sugar moiety, and its phosphate linking group that results in changes in the internucleoside linkage.

As used herein, the term “oligo” or “oligonucleotide” refers to a compound comprising a plurality of linked nucleoside monomers. In certain embodiments, one or more of nucleoside monomers or one or more of the internucleoside linkages are modified.

The terms “deoxynucleoside” and “deoxyribonucleoside” are used interchangeably herein. The terms “deoxynucleotide” and “deoxyribonucleotide” are also used interchangeably herein. As used herein, a “deoxynucleoside” or “deoxynucleotide” is a nucleoside or nucleotide, respectively, that contains a deoxy sugar moiety.

As used herein, the term “duplex DNA” as in “short duplex DNA (sdDNA)” or “asymmetric short duplex DNA (asdDNA)” means a molecule composed of two strands or chains of nucleotide monomers that hybridize with each other to form as duplex oligonucleotides and are caused to contact a cell or administered to a subject, and where the majority, i.e., 50% or more of the linked nucleotide monomers of the key RNA-targeting motifs are deoxyribonucleotide monomers including modified deoxyribonucleotides.

As used herein, the term “motif” means the pattern of chemically distinct regions, e.g., in an antisense strand or a sense strand.

As used herein, the term “immediately adjacent” means there are no intervening elements in between two elements, for example, between regions, segments, nucleotides and/or nucleosides.

As used herein, the term “modified nucleotide” means a nucleotide having at least one modified sugar moiety, modified internucleoside linkage, and/or modified nucleobase.

As used herein, the term “modified nucleoside” means a nucleoside having at least one modified sugar moiety, and/or modified nucleobase.

As used herein, the term “modified oligonucleotide” means an oligonucleotide comprising at least one modified nucleotide.

As used herein, the term “naturally occurring internucleoside linkage” means a 3′ to 5′ phosphodiester linkage.

As used herein, the term “modified internucleoside linkage” refers to a substitution or any change from a naturally occurring internucleoside bond. For example, a phosphorothioate linkage is a modified internucleoside linkage.

As used herein, the term “natural sugar moiety” means a sugar naturally found in DNA (2-H) or RNA (2-OH).

As used herein, the term “modified sugar” refers to a substitution or change from a natural sugar. For example, a 2′-O-methoxyethyl modified sugar is a modified sugar.

As used herein, the term “bicyclic sugar” means a furosyl ring modified by the bridging of two non-geminal ring atoms. A bicyclic sugar is a modified sugar.

As used herein, the term “bicyclic nucleic acid,” “BNA,” “bicyclic nucleoside,” or “bicyclic nucleotide” refers to a nucleoside or nucleotide where the furanose portion of the nucleoside or nucleotide includes a bridge connecting two carbon atoms on the furanose ring, thereby forming a bicyclic ring system.

As used herein, the term “2′-O-methoxyethyl” (also 2′-MOE, 2′-O(CH₂)₂—OCH₃ and 2′-O-(2-methoxyethyl)) refers to an O-methoxy-ethyl modification of the 2′ position of a furosyl ring. A 2′-O-methoxyethyl modified sugar is a modified sugar. As used herein, the term “2′-O-methoxyethyl nucleotide” (also 2′-MOE RNA) means a modified nucleotide comprising a 2′-O-methoxyethyl modified sugar moiety.

As used herein, the term “modified nucleobase” refers to any nucleobase other than adenine, cytosine, guanine, thymidine, or uracil. For example, 5-methylcytosine is a modified nucleobase. In contrast, an “unmodified nucleobase,” as used herein, means the purine bases adenine (A) and guanine (G), and the pyrimidine bases thymine (T), cytosine (C), and uracil (U).

As used herein, the term “5-methylcytosine” means a cytosine modified with a methyl group attached to the 5 position. A 5-methylcytosine is a modified nucleobase.

As used herein, “RNA-like nucleotide” means a modified nucleotide that adopts a northern configuration and functions like RNA when incorporated into an oligonucleotide. RNA-like nucleotides include but are not limited to 2′-endo furanosyl nucleotides, bridged nucleic acid (BNA), LNA, cEt, 2′-O-methylated nucleic acid, 2′-O-methoxyethylated (2′-MOE) nucleic acid, 2′-fluorinated nucleic acid, 2′-O-aminopropylated (2′-AP) nucleic acid, hexitol nucleic acid (HNA), cyclohexane nucleic acid (CeNA), peptide nucleic acid (PNA), glycol nucleic acid (GNA), threose nucleic acid (TNA), morpholino nucleic acid, tricyclo-DNA (tcDNA) and RNA surrogates.

As used herein, “DNA-like nucleotide” means a modified nucleotide that functions like DNA when incorporated into an oligonucleotide. DNA-like nucleotides include but are not limited to 2′-deoxy-2′-fluoroarabinose (FANA) nucleotides and DNA surrogates.

As used herein, “non-coding RNA” means an RNA molecule that is not translated into a protein. Examples of non-coding RNAs include transfer RNAs (tRNAs) and ribosomal RNAs (rRNAs), as well as small non-coding RNAs and the long ncRNAs (lncRNAs). As used herein, examples of “small non-coding RNA” includes, but are not limited to, microRNAs (miRNAs), asRNA, pre-miRNAs, pri-miRNAs, piRNAs, snoRNAs, snRNAs, exRNAs, scaRNAs and mimics of any of the foregoing. As used herein, “lncRNA”, “long non-coding RNA” are transcribed RNA molecules containing greater than 200 nucleotides that do not code for protein. LncRNAs can also be subjected to common post-transcriptional modifications, including 5′-capping, 3′-polyadenylation, and splicing. Generally, lncRNA are a diverse class of molecules that play a variety of roles in modulation of gene and genome function. For example, lncRNAs are known to regulate gene transcription, translation, and epigenetic regulation. Examples of lncRNAs include, but are not limited to Konqlotl, Xlsirt, Xist, ANRIL, NEAT1, NRON, DANCR, OIP5-AS1, TUG1, CasC7, HOTAIR and MALATI. As used herein, “splice” or “splicing” refers to a natural process that removes unnecessary regions of RNA and reforms the RNA. An example of modulation of RNA target function by oligonucleotides including duplex thereof is modulation of non-coding RNA function. In some embodiments, an oligonucleotide or an oligonucleotide duplex is designed to target one of the foregoing small non-coding RNAs. In some embodiments, the oligonucleotide or oligonucleotide duplex is designed to target miRNA. In some embodiments, the oligonucleotide or oligonucleotide duplex is designed to target pre-miRNA. In some embodiments, the oligonucleotide or oligonucleotide duplex is designed to target pri-miRNA. In some embodiments, the oligonucleotide or oligonucleotide duplex is designed to target lncRNA. In some embodiments, the oligonucleotide or oligonucleotide duplex is designed to target splice.

The term “isolated” or “purified” as used herein refers to a material being substantially or essentially free from components that normally accompany it in its native state. Purity and homogeneity are typically determined using analytical chemistry techniques such as polyacrylamide gel electrophoresis or high-performance liquid chromatography.

The term “interspersed” as used herein refers to having a different kind of moiety at an adjacent space, for instance, by a different kind of nucleotide or nucleotide analogue, a different modification on the same kind of nucleotide or nucleotide analogue. In various embodiments of the invention, an “interspersed segment of ribonucleotide monomer(s) (ISR)” refers to a section in an oligonucleotide strand where one or multiple ribonucleotide(s) are connected to at least one moiety that is a different kind from said ribonucleotide(s). For example: if said ribonucleotide(s) are unmodified, then a different kind of moiety may be a deoxynucleotide or an analog thereof, a modified deoxynucleotide, a modified ribonucleotide, or a ribonucleotide analog. if said ribonucleotide(s) are modified, then a different kind of moiety may be a deoxynucleotide or an analogue thereof, a modified deoxynucleotide, an unmodified ribonucleotide, a differently modified ribonucleotide, or a different kind of ribonucleotide analog.

As used herein, “modulating”, “regulating” and its grammatical equivalents refer to either increasing or decreasing (e.g., silencing), in other words, either up-regulating or down-regulating. As used herein, “gene silencing” refers to reduction of gene expression and may refer to a reduction of gene expression about 20%, 30%, 40%, 50%, 60%, 70%, 80%, 90% or 95% of the targeted gene.

As used herein, the terms “inhibiting”, “to inhibit” and their grammatical equivalents, when used in the context of a bioactivity, refer to a down-regulation of the bioactivity, which may reduce or eliminate the targeted function, such as the production of a protein or the phosphorylation of a molecule. In particular embodiments, inhibition may refer to a reduction of about 20%, 30%, 40%, 50%, 60%, 70%, 80%, 90% or 95% of the targeted activity. When used in the context of a disorder or disease, the terms refer to success at preventing the onset of symptoms, alleviating symptoms, or eliminating the disease, condition or disorder.

As used herein, the term “substantially complementary” or “complementary” refers to complementarity in a base-paired, double-stranded region between two chains of linked nucleosides and not any single-stranded region such as a terminal overhang or a gap region between two double-stranded regions. The complementarity does not need to be perfect; there may be any number of base pair mismatches, for example, between the two chains of linked nucleosides. However, if the number of mismatches is so great that no hybridization can occur under even the least stringent hybridization conditions, the sequence is not a substantially complementary sequence. Specifically, when two sequences are referred to as “substantially complementary” herein, it means that the sequences are sufficiently complementary to each other to hybridize under the selected reaction conditions. The relationship of nucleic acid complementarity and stringency of hybridization sufficient to achieve specificity is well known in the art. Two substantially complementary strands can be, for example, perfectly complementary or can contain from 1 to many mismatches so long as the hybridization conditions are sufficient to allow, for example discrimination between a pairing sequence and a non-pairing sequence. Accordingly, substantially complementary sequences can refer to sequences with base-pair complementarity of at least, 70%, 75%, 80%, 85%, 86%, 87%, 88%, 89%, 90%, 91%, 92%, 93%, 94%, 95%, 96%, 97%, 98%, 99%, or 100%, or any number in between, in a double-stranded region.

As used herein, “fully complementary” or “100% complementary” means each nucleobase of a nucleobase sequence of a first strand of linked nucleosides has a complementary nucleobase in a second nucleobase sequence of a second strand of linked nucleosides. In certain embodiments, a first strand of linked nucleosides is an antisense compound and a second strand of linked nucleosides is a target nucleic acid. In certain embodiments, a first strand of linked nucleosides is a sense compound and a second strand of linked nucleosides is an antisense compound or vice versa.

As used herein, the term “targeting region” refers to a region in an oligonucleotide strand that is substantially or fully complementary to another oligonucleotide strand such that the two strands, under the right conditions, hybridize or anneal to each other at such targeting region. For example, an antisense strand can include a targeting region through which it can hybridize with a targeted mRNA.

The terms “administer,” “administering,” or “administration” are used herein in their broadest sense. These terms refer to any method of introducing to a subject a compound or pharmaceutical composition described herein and can include, for example, introducing the compound systemically, locally, or in situ to the subject. Thus, a compound of the present disclosure produced in a subject from a composition (whether or not it includes the compound) is encompassed in these terms. When these terms are used in connection with the term “systemic” or “systemically,” they generally refer to in vivo systemic absorption or accumulation of the compound or composition in the blood stream followed by distribution throughout the entire body.

The terms “effective amount” and “therapeutically effective amount” refer to that amount of a compound or pharmaceutical composition described herein that is sufficient to affect the intended result including, but not limited to, disease treatment, as illustrated below. In some embodiments, the “therapeutically effective amount” is the amount that is effective for detectable killing or inhibition of the growth or spread of cancer cells, the size or number of tumors, and/or other measure of the level, stage, progression and/or severity of the cancer. In some embodiments, the “therapeutically effective amount” refers to the amount that is administered systemically, locally, or in situ (e.g., the amount of compound that is produced in situ in a subject). The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose may vary depending on, for example, the particular pharmaceutical composition, subject and their age and existing health conditions or risk for health conditions, the dosing regimen to be followed, the severity of the disease, whether it is administered in combination with other agents, timing of administration, the tissue to which it is administered, and the physical delivery system in which it is carried.

The term “cancer” in a subject refers to the presence of cells possessing characteristics typical of cancer-causing cells, such as uncontrolled proliferation, immortality, metastatic potential, rapid growth and proliferation rate, and certain morphological features. Often, cancer cells will be in the form of a tumor or mass, but such cells may exist alone within a subject, or may circulate in the blood stream as independent cells, such as leukemic or lymphoma cells. Examples of cancer as used herein include, but are not limited to, lung cancer, pancreatic cancer, bone cancer, skin cancer, head or neck cancer, cutaneous or intraocular melanoma, breast cancer, uterine cancer, ovarian cancer, peritoneal cancer, colon cancer, rectal cancer, colorectal adenocarcinoma, cancer of the anal region, stomach cancer, gastric cancer, gastrointestinal cancer, gastric adenocarcinoma, adrenocorticoid carcinoma, uterine cancer, carcinoma of the fallopian tubes, carcinoma of the endometrium, carcinoma of the vagina, carcinoma of the vulva, Hodgkin's Disease, esophageal cancer, gastroesophageal junction cancer, gastroesophageal adenocarcinoma, chondrosarcoma, cancer of the small intestine, cancer of the endocrine system, cancer of the thyroid gland, cancer of the parathyroid gland, cancer of the adrenal gland, sarcoma of soft tissue, Ewing's sarcoma, cancer of the urethra, cancer of the penis, prostate cancer, bladder cancer, testicular cancer, cancer of the ureter, carcinoma of the renal pelvis, mesothelioma, hepatocellular cancer, biliary cancer, kidney cancer, renal cell carcinoma, chronic or acute leukemia, lymphocytic lymphomas, neoplasms of the central nervous system (CNS), spinal axis tumors, brain stem glioma, glioblastoma multiforme, astrocytomas, schwannomas, ependymomas, medulloblastomas, meningiomas, squamous cell carcinomas, pituitary adenomas, including refractory versions of any of the above cancers, or a combination of one or more of the above cancers. Some of the exemplified cancers are included in general terms and are included in this term. For example, urological cancer, a general term, includes bladder cancer, prostate cancer, kidney cancer, testicular cancer, and the like; and hepatobiliary cancer, another general term, includes liver cancers (itself a general term that includes hepatocellular carcinoma or cholangiocarcinoma), gallbladder cancer, biliary cancer, or pancreatic cancer. Both urological cancer and hepatobiliary cancer are contemplated by the present disclosure and included in the term “cancer.”

The term “pharmaceutical composition” is a formulation containing the active ingredient, e.g., the molecule or composition disclosed herein, in a form suitable for administration to a subject, often in mixture with other substances, e.g., a pharmaceutical carrier such as a sterile aqueous solution. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient in a unit dose of composition is an effect the amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, intranasal, and the like. Dosage forms for the topical or transdermal administration of an asdDNA of this invention include powders, sprays, ointments, pastes, creams, lotions, gels, solutions, patches and inhalants.

The term “pharmaceutical agent” means a substance that provides a therapeutic benefit when administered to an individual.

The term “pharmaceutically acceptable carrier” means a medium or diluent that does not interfere with the structure of the compound. Certain of such carriers enable pharmaceutical compositions to be formulated as, for example, tablets, pills, dragees, capsules, liquids, gels, syrups, slurries, suspension and lozenges for the oral ingestion by a subject. Certain of such carriers enable pharmaceutical compositions to be formulated for injection, infusion or topical administration. For example, a pharmaceutically acceptable carrier can be a sterile aqueous solution.

The term “pharmaceutically acceptable derivative” encompasses derivatives of the compounds described herein such as solvates, hydrates, esters, prodrugs, polymorphs, isomers, isotopically labelled variants, pharmaceutically acceptable salts and other derivatives known in the art.

The term “pharmaceutically acceptable salts” means physiologically and pharmaceutically acceptable salts of compounds, i.e., salts that retain the desired biological activity of the parent compound and do not impart undesired toxicological effects thereto. The term “pharmaceutically acceptable salt” or “salt” includes a salt prepared from reacting the parent compound with pharmaceutically acceptable non-toxic acids or bases, including inorganic or organic acids and bases. Pharmaceutically acceptable salts of the compounds described herein may be prepared by methods well-known in the art. For a review of pharmaceutically acceptable salts, see Stahl and Wermuth, Handbook of Pharmaceutical Salts: Properties, Selection and Use (Wiley-VCH, Weinheim, Germany, 2002). Pharmaceutically acceptable salt can include, but is not limited to, acid addition salts including hydrochlorides, hydrobromides, phosphates, sulphates, hydrogen sulphates, alkylsulphonates, arylsulphonates, acetates, benzoates. citrates, maleales, fumarates, succinates, lactates, and tartrates; alkali metal cations such as Na, K, Li, alkali earth metal salts such as Mg or Ca, or organic amine salts. In particular, sodium salts of oligonucleotides have proven to be useful and are well accepted for therapeutic administration to humans. Accordingly, in one embodiment, the compounds described herein are in the form of a sodium salt.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

Terms such as “treating” or “treatment” or “to treat” or “alleviating” or “to alleviate” as used herein refer to both (1) therapeutic measures that cure, slow down, lessen symptoms of, and/or halt progression of a diagnosed pathologic condition or disorder and (2) prophylactic or preventative measures that prevent or slow the development of a targeted pathologic condition or disorder. Thus, those in need of treatment include those already with the disorder; those prone to have the disorder; and those in whom the disorder is to be prevented. A subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer into soft tissue and bone; inhibition of or an absence of tumor metastasis; inhibition or an absence of tumor growth; relief of one or more symptoms associated with the specific cancer; reduced morbidity and mortality; and improvement in quality of life.

The term “carrier” as used herein means a pharmaceutically acceptable material, composition or vehicle, such as, for example, a liquid or solid filler, diluent, excipient, solvent or encapsulating material involved in or capable of carrying or transporting the subject pharmaceutical compound from one organ, or portion of the body, to another organ, or portion of the body. Each carrier must be “acceptable” in the sense of being compatible with the other ingredients of the formulation and not injurious to the patient. Non-limiting examples of pharmaceutically acceptable carriers, carriers, and/or diluents include: sugars, such as lactose, glucose and sucrose; starches, such as corn starch and potato starch; cellulose and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers, and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

### 2. CERTAIN EMBODIMENTS

Certain embodiments of the present invention provide a duplex composition where the antisense and sense strands are both made of linked nucleoside monomers. Fifty percent or more of nucleoside monomers in the key RNA-targeting motifs are deoxyribonucleoside monomers, or fifty percent or more nucleobase pair in one strand of the double-stranded region of the asdDNA molecule comprises deoxyribonucleoside monomer, and some of the deoxyribonucleoside monomers contained therein and/or the internucleoside linkage(s) may be modified from those found in natural DNAs. The duplex DNA molecule of the invention further includes ribonucleoside monomers in one or more interspersed segments of ribonucleotide monomer(s) (“ISRs”). One or more ISRs may be found in either the antisense or the sense strand, or both. In some embodiments, each ISR independently consists of 1 ribonucleotide monomer, or 2, 3, 4, or 5 contiguous ribonucleotide monomers. In some embodiments, an ISR has at least two contiguous and linked ribonucleotide monomers.

Both the antisense and sense strands of the duplex molecule of the invention are relatively short, with the antisense strand being longer of the two, hence an “asymmetric short duplex DNA (asdDNA).”

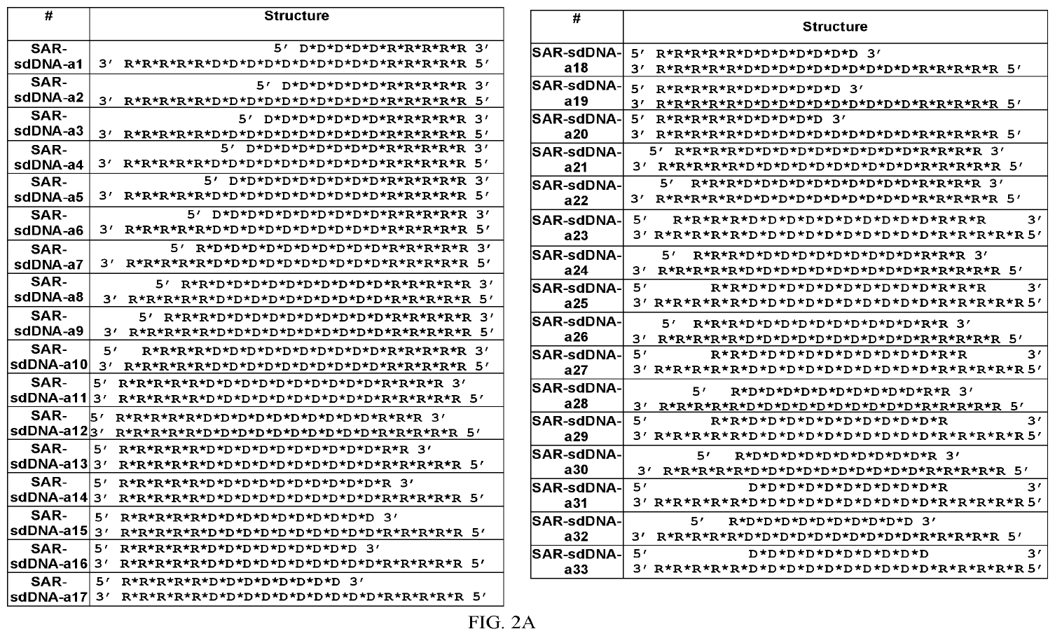

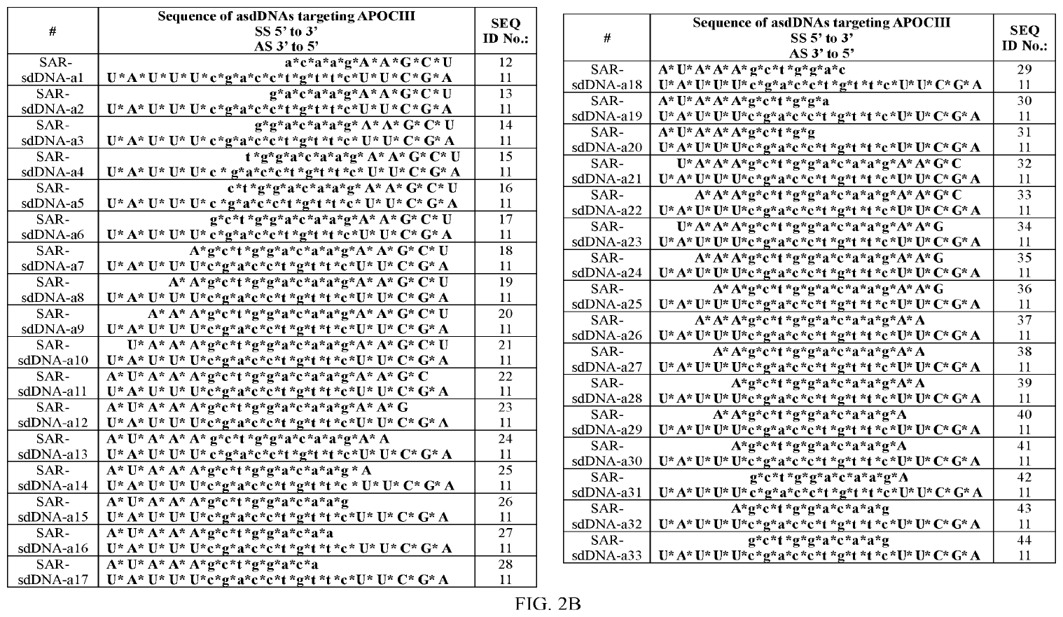

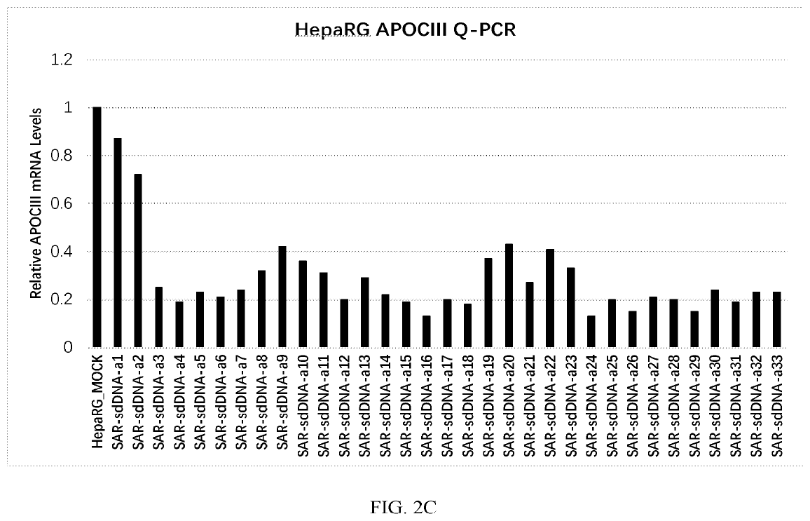

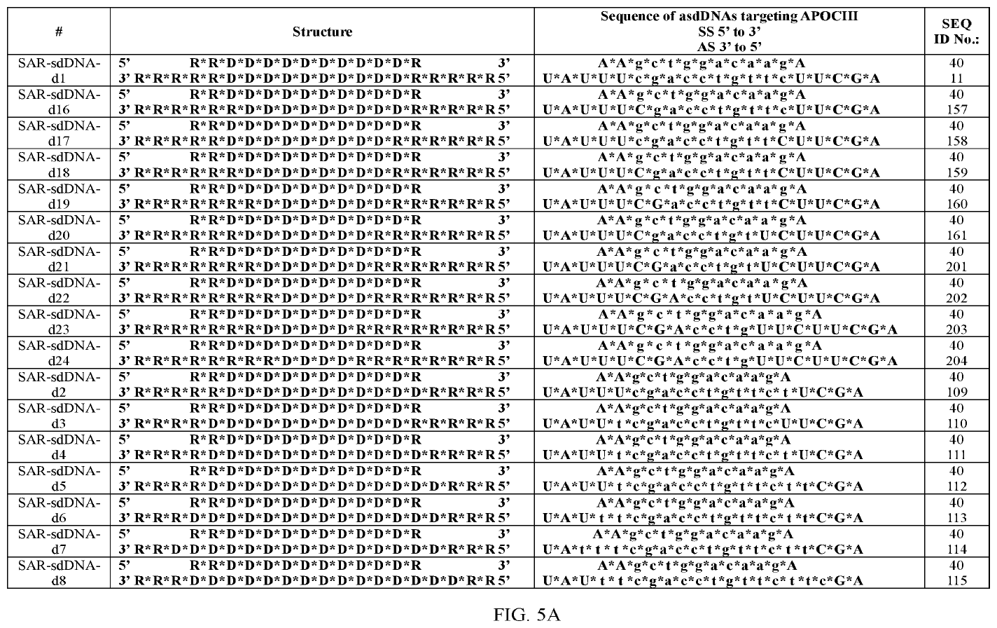

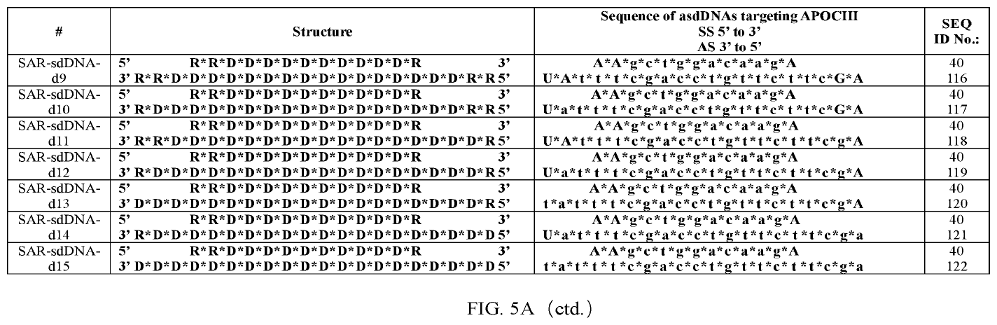

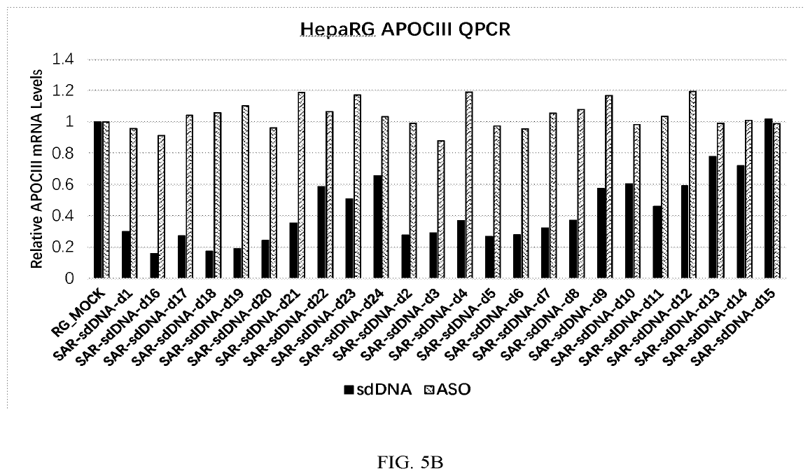

Exemplary structures of the duplex molecule of the invention are shown in FIG. 2A, 3A, 4A, 5A, 6A, 7A, 17, 18, 19, 20 where ISRs are found in both strands or is only found in the longer antisense strand in almost all duplexes, except for the last one in FIG. 5A where ISR is only found in the shorter sense strand, which is also the only one shows relatively low gene silencing activity at 100 pm.

In some embodiments, the length asymmetry between antisense and sense strands leads to at least one overhang in the antisense strand at its 5′ end (e.g., first three on the right side in FIG. 2A) or its 3′ end (e.g., first ten on the left side in FIG. 2A) with the other end being blunt or recessed. In other embodiments, there are overhangs on both ends of the antisense strand (e.g., last thirteen on the right side in FIG. 2A).

The composition of the invention can be used for modulating gene expression or function in eukaryotic cell in at least three ways: (i) one kind of asdDNA molecules are caused to contact a cell or administered to a subject; (ii) different kinds of asdDNA molecules are caused to contact a cell or administered to a subject separately at different times; (ii) different kinds of asdDNA molecules are caused to contact a cell or administered to a subject simultaneously.

In certain embodiments, the antisense oligonucleotide strand includes a nucleobase sequence region, called a “targeting region,” that is at least 70%, 75%, 80%, 85%, 86%, 87%, 88%, 89%, 90%, 91%, 92%, 93%, 94%, 95%, 96%, 97%, 98%, 99%, or 100% complementary to the target segment of a target gene to which it is targeted, including an mRNA and a non-coding RNA. In certain embodiments, the antisense oligonucleotide has a nucleobase sequence comprising a fully complementary sequence of the target segment of a target gene to which it is targeted. In certain embodiments, the antisense oligonucleotide has a nucleobase sequence comprising no more than 1, 2 or 3 mismatch(es) when hybridized to the target segment of a target gene to which it is targeted. In certain embodiments, the target gene is selected from mRNA or non-coding RNA that are implicated in a mammalian disease. In some embodiments, at least one ISR is disposed in a targeting region of the antisense strand. In certain embodiments, an ISR is positioned at or near (i.e., within one third of the length of strand, which means, e.g., for a strand that is about 21 nucleobases long, within 7 nucleobases counting the terminal) the 5′ end of the antisense strand. Alternately, the ISR is at or near (i.e., within one third of the length of strand, which means, e.g., for a strand that is about 21 nucleobases long, within 7 nucleobases counting the terminal) the 3′ end of the antisense strand. In some embodiments, an ISR or at least part of the ISR is also positioned at a more central part of the antisense strand, i.e., in the middle third of the length, which means, e.g., for a strand that is about 21 nucleobases long, more than 7 nucleobases away from both terminals of the antisense strand.

In various embodiments, the first or antisense strand has a backbone length of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 linked nucleotide monomers, or equivalents thereof, or of a range bracketed by any two of the above values (both range endpoints included). For example, some of the ranges of the first antisense strand include: 8-50 nucleotide monomers; 8-36 nucleotide monomers; 8-33 nucleotide monomers; 10-30 nucleotide monomers; 10-29 nucleotide monomers; 12-29 nucleotide monomers; 12-28 nucleotide monomers; 12-26 nucleotide monomers; 12-25 nucleotide monomers; 13-25 nucleotide monomers; 13-24 nucleotide monomers; 13-23 nucleotide monomers; 15-23 nucleotide monomers; 10-36 nucleotide monomers; 12-36 nucleotide monomers; 12-32 nucleotide monomers; 14-36 nucleotide monomers; and at least 8 nucleotide monomers.

In certain embodiments, the antisense oligonucleotide strand is 10 to 36 (both range endpoints included) nucleotide monomers in length. In other words, antisense strands are from 10 to 36 (both range endpoints included) linked nucleobase monomers. In other embodiments, the antisense strand comprises a modified oligonucleotide consisting of 8 to 100, 10 to 80, 12 to 50, 14 to 30, 15 to 23, 16 to 22, 16 to 21, or 20 (both range endpoints included) linked nucleobases.

In certain embodiments, the antisense oligonucleotide consists of 13-23 (both range endpoints included) linked nucleoside monomers. In certain embodiments, the antisense oligonucleotide consists of 23 linked nucleoside monomers. In certain embodiments, the antisense oligonucleotide consists of 20 linked nucleoside monomers. In certain embodiments, the antisense oligonucleotide consists of 16 linked nucleoside monomers.

In certain embodiments, the sense strand includes a nucleobase sequence that is substantially complementary to the antisense strand and is at least 70%, 75%, 80%, 85%, 86%, 87%, 88%, 89%, 90%, 91%, 92%, 93%, 94%, 95%, 96%, 97%, 98%, 99%, or 100% complementary to a sequence of a linked region of the antisense oligonucleotide, as measured over the entire nucleobase sequence of the sense strand. These substantially complementary sequences from both strands form one or more double-stranded regions. In certain embodiments, the sense strand has a nucleobase sequence comprising fully complementary sequence of a linked region of the antisense strand. In some embodiments, at least one ISR is disposed in a double-stranded region of the sense strand.

In certain embodiments, an ISR is positioned at or near (within 33% of total number of nucleobases from a terminal counting said terminal) the 5′ end of the sense strand, or at or near (within 33% of total number of nucleobases from a terminal counting said terminal) the 3′ end of the sense strand. In some embodiments, an ISR or at least a part of the ISR is also positioned at a more central part of the sense strand, i.e., more than 33% of total number of nucleobases away from both terminals of the sense strand. In some embodiments, an ISR is not necessary to be deposited in the sense strand.

In a feature, the sense oligonucleotide strand has a length shorter than the antisense oligonucleotide strand. In certain embodiments, the sense strand has a length from about half to about full length of the antisense strand. In certain embodiments, the sense strand has a length from about one quarter to about full length of the antisense strand. In certain embodiments, the sense strand is 6 to 29 (both range endpoints included) nucleotide monomers in length. In other words, those sense strands are from 6 to 29 (both range endpoints included) linked nucleobases. In other embodiments, the sense strand comprises an oligonucleotide consisting of 13, 4 to 30, 6 to 16, 10 to 20, or 12 to 16 (both range endpoints included) linked nucleobases. In certain such embodiments, the sense strand comprises an oligonucleotide consisting of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 and 49 linked nucleobases in length, or a range defined by any two of the above values (both range endpoints included). In some embodiments, the sense strand is a sense oligonucleotide.

In one feature, the second or sense strand has a backbone length shorter than the first strand or the antisense strand by a number of nucleotide monomers as follows: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38. In various embodiments, the second or sense strand has a backbone length of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48 or 49 linked nucleotide monomers, or equivalents thereof, or of a range bracketed by any two of the above values (both range endpoints included). In certain embodiments, for example, some of the ranges of the second, sense strand include: 6-49 nucleotide monomers; 8-46 nucleotide monomers; 8-35 nucleotide monomers; 9-35 nucleotide monomers; 10-46 nucleotide monomers; 10-40 nucleotide monomers; 10-34 nucleotide monomers; 8-32 nucleotide monomers; 8-30 nucleotide monomers; 8-29 nucleotide monomers; 9-29 nucleotide monomers; 9-26 nucleotide monomers; 9-25 nucleotide monomers; 10-29 nucleotide monomers; 10-28 nucleotide monomers; 10-26 nucleotide monomers; 10-25 nucleotide monomers; 11-24 nucleotide monomers; 11-23 nucleotide monomers; 12-23 nucleotide monomers; 13-23 nucleotide monomers; 12-22 nucleotide monomers tides; 13-23 nucleotide monomers; 15-23 nucleotide monomers tides; and at least 6 nucleotide monomers. In certain embodiments, the second strand can have a backbone length of any nucleotide monomers when duplex can be formed with the first strand thermodynamically.

In certain embodiments, the sense strand is 1, 2, 3, 4, 5, 6, 7, 8, 9 or 10 nucleotide monomers shorter than the antisense strand. In certain embodiments, the sense strand consists of 8-23 (both range endpoints included) linked nucleoside monomers. In certain embodiments, the sense strand consists of 13 linked nucleoside monomers. In certain embodiments, the sense strand consists of 15 linked nucleoside monomers.

In various embodiments of the invention, the two ends of the antisense strand are one of the following configurations: a 3′-overhang and a 5′-overhang; a 3′-overhang and a blunt end at 5′ end; a 5′-overhang and a blunt end at 3′ end; 3′-overhang and 5′ recessed-end; or 5′-overhang and 3′ recessed-end.

In certain embodiments, the 3′-overhang of the antisense strand has a length of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide monomers. In various embodiments, the 3′-overhang of the antisense strand has a length of 1-15, 1-10, 1-8, or 1-5 nucleotide monomers (both range endpoints included).

In certain embodiments, the 5′-overhang of the antisense strand has a length of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide monomers. In various embodiments, the 5′-overhang of the antisense strand has a length of 1-15, 1-10, 1-8, or 1-5 nucleotide monomers (both range endpoints included).

In an embodiment of the invention, the antisense strand has a 3′-overhang of 1-15 (both range endpoints included) nucleotide monomers and a 5′-overhang of 1-15 (both range endpoints included) nucleotide monomers. In another embodiment, the antisense strand has a 3′-overhang of 1-26 (both range endpoints included) nucleotide monomers and a 5′ blunt end or a 5′ recessed end. In yet another embodiment, the antisense strand has a 5′-overhang of 1-26 (both range endpoints included) nucleotide monomers and a 3′ blunt end or a 3′ recessed end.

In various embodiments of the invention, the two ends of the second (sense) strand are one of the following configurations: a 3′-overhang and a 5′ recessed-end; a 5′-overhang and 3′ recessed-end; a 3′ blunt-end and a 5′ recessed-end; a 5′ blunt-end and a 3′ recessed-end; or a 3′ recessed-end and a 5′ recessed-end. In certain embodiments, the 3′-overhang of the second strand has a length of 1, 2, 3, 4, 5, 6, 7, 8, 9, 10, 11, 12, 13, 14, 15, 16, 17, 18, 19, 20, 21, 22, 23, 24, or 25 nucleotide monomers. In various embodiments, the 3′-overhang of the second strand has a length of 1-15, 1-10, 1-8, or 1-5 nucleotide monomers (both range endpoints included). In certain embodiments, the 5′-overhang of the second strand has a length of 1, 2, 3, 4, 5, 6, 7, 8, 9, 10, 11, 12, 13, 14, 15, 16, 17, 18, 19, 20, 21, 22, 23, 24, or 25 nucleotide monomers. In various embodiments, the 5′-overhang of the second strand has a length of 1-15, 1-10, 1-8, or 1-5 nucleotide monomers.

In the asdDNA molecule of the invention, at least one nucleotide monomer in the first strand and/or the second strand can be a modified nucleotide or nucleotide analogue, e.g., a sugar-, backbone-, and/or base-modified nucleotide. In an embodiment, such a backbone-modified nucleotide has at least a modification in an internucleoside linkage, e.g., to include at least one of a nitrogen or sulphur heteroatom. In some embodiments, the modified internucleoside linkage is or includes: phosphorothioate (P═S) group, phosphotriesters, methylphosphonates, or phosphoramidate.

In certain embodiments, the antisense strand and/or the sense strand comprises at least one modified internucleoside linkage. Such modified internucleoside linkage may be between two deoxyribonucleoside monomers, two ribonucleoside monomers, or one deoxyribonucleoside monomer and one ribonucleoside monomer. Alternately, the phosphate group on at least one of the terminal nucleoside monomers may be modified. In certain embodiments, the internucleoside linkage is a phosphorothioate internucleoside linkage. In certain embodiments, the internucleoside linkage is a thio-phosphoramidate internucleoside linkage. In certain embodiments, each internucleoside linkage of the oligonucleotide strand is a phosphorothioate internucleoside linkage. In certain embodiments, all the internucleoside linkages in a strand, antisense or sense or both, are phosphorothioate internucleoside linkages, or a mixture of phosphorothioate and phosphodiester linkages.

In certain embodiments, the antisense strand and/or the sense strand comprises at least one nucleoside monomer having a modified sugar moiety. Such a nucleoside monomer can be a deoxyribonucleoside monomer or a ribonucleoside monomer.

In certain embodiments, the 2′ position of the modified sugar moiety is replaced by a group selected from OR, R, halo, SH, SR, NH₂, NHR, NR₂, or CN, where each R is independently C₁-C₆ alkyl, alkenyl or alkynyl, and halo is F, Cl, Br or I. In some embodiments, the 2′ position of the modified sugar moiety is replaced by a group selected from allyl, amino, azido, thio, O-allyl, O-C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), or O—CH₂—C(═O)—N(Rₗ)—(CH₂)₂—N(Rₘ)(Rₙ), where each Rₗ, Rₘ and Rₙ is, independently, H or substituted or unsubstituted C₁-C₁₀ alkyl. In some embodiments, the modified sugar moiety is selected from the group of 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. In some embodiments, the modified sugar moiety is substituted by bicyclic sugar selected from the group of 4′-(CH₂)—O-2′ (LNA); 4′-(CH₂)—S-2; 4′-(CH₂)₂—O-2′ (ENA); 4′-CH(CH₃)—O-2′ (cEt) and 4′-CH(CH₂OCH₃)—O-2′, 4′-C(CH₃)(CH₃)— O-2′, 4′-CH₂—N(OCH₃)-2′, 4′-CH₂—O—N(CH₃)-2′, 4′-CH₂—N(R)—O-2′, where R is H, C1-C12 alkyl, or a protecting group, 4′-CH₂—C(H)(CH₃)-2′, and 4′-CH₂—C—(═CH₂)-2′.

In some embodiments, the modified sugar moiety is selected from the group of 2′-O-methoxyethyl modified sugar (MOE), a 4′-(CH₂)—O-2′ bicyclic sugar (LNA), 2′-deoxy-2′-fluoroarabinose (FANA), and a methyl(methyleneoxy) (4′-CH(CH₃)—O-2) bicyclic sugar (cEt).

In some embodiments, the antisense strand and/or the sense strand of the molecule of the invention includes at least one nucleotide monomer having a modified nucleobase. Such a nucleoside monomer can be a deoxyribonucleoside monomer or a ribonucleoside monomer.

In some embodiments, the modified nucleobase is selected from the group of: 5-methylcytosine (5-Me-C), inosine base, a tritylated base, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, and 7-deazaguanine and 7-deazaadenine and 3-deazaguanine and 3-deazaadenine.

In a particular embodiment, the modified nucleobase in the molecule of the invention is a 5-methylcytosine. In an embodiment, each cytosine base in the molecule of the invention is 5-methylcytosine. In certain embodiments, the modified nucleobase is a 5-methyluracil. In certain embodiments, each uracil is a 5-methyluracil.

In certain embodiments, either the antisense strand or the sense strand or both of the molecule of the invention comprise linked deoxynucleoside monomers. In certain embodiments, an entire strand, antisense or sense, consists exclusively of linked deoxynucleoside monomers. In certain embodiments, an entire sense strand consists exclusively of linked deoxynucleoside monomers. In a feature, either the antisense strand or the sense strand or both, in addition to the linked deoxynucleoside monomers, further includes an ISR that consist of one or more linked ribonucleoside monomers. In another feature, either the antisense strand or both antisense and sense strand, in addition to the linked deoxynucleoside monomers, further includes an ISR that consist of one or more linked ribonucleoside monomers. Further, there may be even more ISR segments. The ISR can be anywhere in either strand. In some embodiments, one or more ISRs include a terminal nucleoside monomer, or a penultimate terminal nucleoside monomer. In some embodiments, one or more ISRs are inserted in a segment of deoxynucleoside monomers, separating them into multiple segments. In certain embodiments, each of the ISRs independently consists of 1 ribonucleoside monomers, 2, 3, 4, or 5 linked ribonucleoside monomers.

In certain embodiments, at least half of the nucleobases in at least one strand of the double-stranded region are deoxyribonucleotide monomer.

In certain embodiments, at least 50% of nucleotides in one strand in the RNA-targeting part of the double-stranded region are deoxyribonucleotide monomer.

In certain embodiments, the total number of ribonucleotide monomer(s) in the asdDNA molecule is no more than the total number of deoxyribonucleotide monomers in the same asdDNA molecule.

In certain embodiments, at least one or each of the linked ribonucleoside monomers of the ISRs is a modified ribonucleotide or ribonucleotide analog. The ribonucleotide may be modified in the same or a similar way as follows: have a modified internucleoside linkage, a modified sugar moiety and/or a modified nucleobase.

In some embodiments, the sugar moiety of the deoxyribonucleotide monomer is either the sugar moiety of a naturally occurring deoxyribonucleotide (2-H) or 2′-deoxy-2′-fluoroarabinose (FANA).

In some embodiments, the sugar moiety of the ribonucleotide monomer is selected from the group consisting of a naturally occurring ribonucleotide (2-OH), 2′-F modified sugar, 2′-OMe modified sugar, 2′-O-methoxyethyl modified sugar (MOE), a 4′-(CH₂)—O-2′ bicyclic sugar (LNA) and a methyl(methyleneoxy) (4′-CH(CH₃)—O-2) bicyclic sugar (cEt).

In certain embodiments, in the antisense strand, the sense strand or both strands, at least one or each ribonucleoside monomer of each ISR therein has a modified sugar moiety selected from the group of 2′-O-methoxyethyl modified sugar (MOE), a 4′-(CH₂)—O-2′ bicyclic sugar (LNA), and a methyl(methyleneoxy) (4′-CH(CH₃)—O-2) bicyclic sugar (cEt). In certain embodiments, in the antisense strand, the sense strand or both strands, at least one deoxyribonucleoside monomer has a modified sugar moiety of 2′-deoxy-2′-fluoroarabinose (FANA). In certain embodiments, each ribonucleoside monomer of each ISR has a 2′-O-methoxyethyl modified sugar, a 4′-(CH₂)—O-2′ bicyclic sugar, or a methyl(methyleneoxy) (4′-CH(CH₃)—O-2) bicyclic sugar (cEt), where each cytosine is a 5-methylcytosine, where each uracil is a 5-methyluracil, or methyl-pseudouracil, and where each internucleoside linkage is a phosphorothioate linkage.

In certain embodiments, the molecule of the invention has either an antisense strand or a sense strand consisting of deoxynucleoside monomers where each internucleoside linkage is a phosphorothioate linkage. In certain embodiments, the molecule of the invention has either an antisense strand or a sense strand consisting of deoxynucleoside monomers wherein each internucleoside linkage is a natural phosphate linkage without the phosphorothioate modification.

In certain embodiments, the molecule of the invention comprises a sense strand, wherein each nucleotide monomer of the sense strand comprising the same modification as the complementary nucleotide monomer of the antisense strand.

Exemplary structures and exemplary sequences of exemplary molecules of the invention with an antisense oligonucleotide strand and a sense oligonucleotide strand are showed in FIGS. 2A, 3A, 4A, 5A, 6A, 7A, 8A, 9A, 10A, 11A, 12A, 13A, 14A, 15A, 16A, 17, 18, 19, 20 and 21.

In certain embodiments, asymmetric short DNA duplex and at least one ISR in the antisense strand of the duplex molecular enable potent gene silencing. Data shown in all Examples below suggest that a new platform technology based on the asymmetric duplex with an antisense oligodeoxyribonucleotide and at least one ISR in the antisense oligodeoxyribonucleotide enabled extremely potent gene silencing. Further studies on SAR (structure-activity relationship) features of the asdDNA, including length motif, complementary and mismatches, various modification motif, etc., were carried out, which help to define various structure factors that may influence gene silencing activities. Such SAR factors are important for designing optimized gene silencers to target various sequences and structures of more than 100,000 different mRNA in a typical mammalian cell as well as much more non-coding RNAs. Our data on the gene silencing activity and the SAR of asdDNA suggest that gene silencing features of asdDNA are vastly different from siRNA and ASO, indicating a novel and distinct mechanism of gene silencing mechanism which is yet to be identified.

In certain embodiments, the molecule of the invention can be stabilized against degradation, either through at least one chemical modification or a secondary structure. The sense oligonucleotide strand and antisense oligonucleotide strand can have unmatched or imperfectly matched nucleotide monomers. The sense oligonucleotide strand and/or antisense oligonucleotide strand may have one or more nicks (a cut in the nucleic acid backbone), gaps (a fragmented strand with one or more missing nucleotides), and modified nucleotides or nucleotide analogues. Not only can any or all of the nucleotide monomers in the sense and antisense oligonucleotide strands chemically modified, each strand may be conjugated to one or more moieties or ligands to enhance its functionality, for example, with moieties or ligands selected from: peptide, antibody, antibody fragment, polymer, polysaccharide, lipid, hydrophobic moiety or molecule, cationic moiety or molecule, lipophilic compound or moiety oligonucleotide, cholesterol, GalNAc and aptamer.

In certain embodiments, the double-stranded region of the duplex molecule of the invention does not contain any mismatch or bulge, and the two strands are perfectly complementary to each other in the double-stranded region. In another embodiment, the double-stranded region of the duplex contains mismatch and/or bulge.

In certain embodiments, the target is mRNA or non-coding RNA implicated in a mammalian disease. In certain embodiments, the target is mRNA. In certain embodiments, the target is non-coding RNA, such as microRNA and lncRNA. The antisense strand can occupy the target by hybridizing to the target sequence as long as they are substantially complementary to each other, and inactive the target gene.

### 3. UNMATCHED OR MISMATCHED REGIONS

The complementary region between the antisense strand and the sense strand of the present invention can have at least one unmatched or imperfectly matched region containing, e.g., one or more mismatches. In some embodiments, the sense strand of the asdDNA provided in this invention can tolerate three or more (at least 15% of the targeting region) mismatches without any effect on gene silencing activities of the asdDNA. Mismatches in sense strand are sometimes desired for reducing off-target effects or enable other features to the asdDNA.

As is well known to one skilled in the art, it is possible to introduce mismatch bases without eliminating activity. Similarly, the antisense oligonucleotides strands of the asdDNA of the present invention can include unmatched or mismatched region(s). In some embodiments, antisense oligonucleotides strands of the asdDNA of the present invention can tolerate at least three (at least 15% of the targeting region) mismatches while maintaining gene silencing activities of the asdDNA. Mismatches in antisense strand are sometimes desired for reducing off-target effects or enable other features to the asdDNA.

### 4. MODIFICATIONS

A nucleoside monomer is a base-sugar composition. The nucleobase (also known as base) portion of the nucleoside monomer is normally a heterocyclic base moiety. Nucleotide monomers are nucleoside monomers that further include a phosphate group covalently linked to the sugar portion of the nucleoside. For those nucleoside monomers that include a pentofuranosyl sugar, the phosphate group can be linked to the 2′, 3′ or 5′ hydroxyl moiety of the sugar. Oligonucleotides are formed through the covalent linkage of adjacent nucleoside monomers to one another, to form a linear polymeric oligonucleotide. Within the oligonucleotide structure, the phosphate groups are commonly referred to as forming the internucleoside linkages of the oligonucleotide.

Modifications to the asdDNA molecule, antisense strand and/or sense strand of the invention encompass substitutions or changes to internucleoside linkages, sugar moieties, or nucleobases. Modified asdDNA, antisense strand and/or sense strands are in some cases preferred over native forms because of desirable properties such as, for example, increased inhibitory activity, enhanced cellular uptake, enhanced strand affinity, solubility, reduce the non-specific interaction and resistance to RNase degradation or enhanced stability otherwise. Consequently, comparable results can often be obtained with shorter antisense strands that have such chemically modified nucleoside monomers. One or more of the natural nucleotides in the antisense and the sense strands of the invention can be substituted with modified nucleotides or nucleotide analogues. The substitution can take place anywhere in the antisense strand and the sense strand.

The modifications of oligonucleotide molecules have been investigated to improve the stability of various oligonucleotide molecules, including antisense oligonucleotide, ribozyme, aptamer, and RNAi (Chiu and Rana, 2003; (zauderna et al., 2003; de Fougerolles et al., 2007; Kim and Rossi, 2007; Mack, 2007; Zhang et al., 2006; Schrnidt, 2007; Setten R L et al., 2020; (rooke S T et al., 2018; and Roberts T O et al., 2020).

Any stabilizing modification known to a person skilled in the art can be used to improve the stability of the oligonucleotide molecules. Within the oligonucleotide molecules, chemical modifications can be introduced to the phosphate backbone (e.g., phosphorothioate linkages), the sugar (e.g., locked nucleic acids, glycerol nucleic acid, cEt, 2′-MOE, 2′-fluorouridine, 2′-O-methyl), and/or the base (e.g., 2′-fluoropyrimidines).

Several examples of such chemical modifications are summarized in the sections that follow.

In various embodiment, the modified nucleotide or a nucleotide analogue is sugar-, backbone- and/or base-modified nucleotide.

### 4.1 Modified Internucleoside Linkages or Backbone-Modified Nucleotide

The naturally occurring internucleoside linkage of RNA and DNA is a 3′ to 5′ phosphodiester linkage. The asdDNA molecule of the invention having one or more modified, i.e., non-naturally occurring, internucleoside linkages in one or both of its strands are sometimes selected over a corresponding molecule with only naturally occurring internucleoside linkages because of desirable properties such as, for example, enhanced cellular uptake, enhanced affinity for target nucleic acids, and increased stability in the presence of nucleases.

Oligonucleotide strands having modified internucleoside linkages include internucleoside linkages that retain a phosphorus atom as well as internucleoside linkages that do not have a phosphorus atom. In an embodiment, the phosphodiester internucleoside linkage is modified to include at least a nitrogen and/or sulphur heteroatom. Representative phosphorus containing internucleoside linkages include, but are not limited to, phosphodiesters, phosphotriesters, methylphosphonates, phosphoramidate, thio-phosphoramidate and phosphorothioates. Methods of preparation of phosphorous-containing and non-phosphorous-containing linkages are well known.

In one embodiment, a modified nucleotide or nucleotide analogue is a backbone-modified nucleotide. The backbone-modified nucleotide may have a modification in a phosphodiester internucleoside linkage. In a further embodiment, the backbone-modified nucleotide is phosphorothioate internucleoside linkage. In certain embodiments, each internucleoside linkage is a phosphorothioate internucleoside linkage.

### 4.2 Modified Sugar Moieties

The antisense and/or the sense strand of the invention can optionally contain one or more nucleoside monomers where the sugar group has been modified. Such sugar-modified nucleoside monomers may impart enhanced nuclease stability, increased binding affinity, or some other beneficial biological property to the strand. In certain embodiments, nucleoside monomers comprise chemically modified ribofuranose ring moieties. Examples of chemically modified ribofuranose rings include without limitation, addition of substitute groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ are each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′, 2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleoside monomers having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O-C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(Rₗ)—(CH₂)₂—N(Rₘ)(Rₙ), where each Rₗ, Rₘ and Rₙ is, independently, H or substituted or unsubstituted C₁-C₁₀ alkyl.

Bicyclic nucleosides are modified nucleosides having a bicyclic sugar moiety. Examples of bicyclic nucleic acids (BNAs) include without limitation nucleosides comprising a bridge between the 4′ and the 2′ ribosyl ring atoms. In certain embodiments, asdDNA, the antisense strand and/or the sense strand provided herein include one or more BNA nucleosides wherein the bridge comprises one of the formulas: 4′-(CH₂)—O-2′ (LNA); 4′-(CH₂)—S-2; 4′-(CH₂)₂—O-2′ (ENA); 4′-CH(CH₃)—O-2′ and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see PCT/US2008/068922 published as WO/2009/006478, published Jan. 8, 2009); 4′-CH₂—N(OCH₃)-2′ (and analogs thereof see PCT/US2008/064591 published as WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O—2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et al., J. Org. Chem., 2009, 74, 118-134); and 4′-CH₂—C—(═CH₂)-2′ (and analogs thereof see PCT/US2008/066154 published as WO 2008/154401, published on Dec. 8, 2008).

In certain embodiments, bicyclic nucleosides include, but are not limited to, (A) α-L-methyleneoxy (4′-CH₂—O-2) BNA (B) β-D-methyleneoxy (4′-CH₂—O-2) BNA (C) ethyleneoxy (4′-(CH₂)₂—O-2′) BNA, (D) aminooxy (4′-CH₂—O—N(R)-2′) BNA, (E) oxyamino (4′-CH₂—N(R)—O-2) BNA, (F) methyl(methyleneoxy) (4′-CH(CH₃)—O-2) BNA (also referred to as constrained ethyl or cEt), (G) methylene-thio (4′-CH₂—S-2′) BNA, (H) methylene-amino (4′-CH₂—N(R)-2′) BNA, (I) methyl carbocyclic (4′-CH₂—CH(CH₃)-2) BNA, (J) propylene carbocyclic (4′-(CH₂)₃-2′) BNA, and (K) vinyl BNA.

In certain embodiments, a modified nucleotide or a nucleotide analogue is a sugar-modified ribonucleotide, in which the 2′-OH group is replaced by a group selected from: H, OR, R, halo, SH, SR, NH₂, NHR, NR₂, and CN, where each R is independently selected from the group consisting of: C₁-C₆ alkyl, alkenyl and alkynyl, and halo is selected from the group of F, Cl, Br and I. In certain embodiments, the sugar-modified ribonucleotide is selected from the group of 2′-OMe modified nucleotide, 2′-F modified nucleotide, 2′-O-methoxyethyl (2′MOE) modified nucleotide, LNA (Locked nucleic acid) modified nucleotide, GNA (Glycerol nucleic acid) modified nucleotide, and cEt (Constrained ethyl) modified nucleotide.

Chemical modifications at the 2′ position of the ribose, such as 2′-O-methylpurines and 2′-fluoropyrimidines, which increases resistance to endonuclease activity in serum, can be adopted to stabilize the molecules of the present invention. The position for the introduction of the modification should be carefully selected to avoid significantly reducing the silencing/regulating of potency of the molecule. In certain embodiments, the first nucleotide monomer adjacent to the 5′-terminal nucleotide monomer of the antisense strand is a 2′-flouro-ribonucleotide.

### 4.3 Modified Nucleobases

The antisense strand and/or the sense strand in the asdDNA molecule can also have nucleobase (or base) modifications or substitutions. Nucleobase (or base) modifications or substitutions are structurally distinguishable from, yet functionally interchangeable with, naturally occurring or synthetic unmodified nucleobases. Both natural and modified nucleobases are capable of participating in hydrogen bonding. Such nucleobase modifications may impart nuclease stability, binding affinity or some other beneficial biological property to the asdDNA molecule. Modified nucleobases include synthetic and natural nucleobases such as, for example, 5-methylcytosine (5-Me-C). Certain nucleobase substitutions, including 5-methylcytosine substitutions, are particularly useful for increasing the binding affinity of the antisense and the sense strands. For example, 5-methylcytosine substitutions have been shown to increase nucleic acid duplex stability by 0.6-1.2° ° C. (Sanghvi, Y. S., Crooke, S. T. and Lebleu, B., eds., Antisense Research and Applications, CRC Press, Boca Raton, 1993, pp. 276-278).

Additional modified nucleobases include and are not limited to: 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 1-methyl pseudouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine.

Heterocyclic base moieties may include those in which the purine or pyrimidine base is replaced with other heterocycles, for example 7-deaza-adenine, 7-deazaguanosine, 2-aminopyridine and 2-pyridone. Nucleobases that are particularly useful for increasing the binding affinity of antisense and the sense strands include 5-substituted pyrimidines, 6-azapyrimidines and N-2, N-6 and O-6 substituted purines, including 2 aminopropyladenine, 5-propynyluracil and 5-propynylcytosine.

In certain embodiments, a modified nucleotide or a nucleotide analogue is a base-modified nucleotide. In an embodiment, a modified nucleotide or a nucleotide analogue has an unusual base or a modified base. In certain embodiments, the modified base is a 5-methylcytosine (5′-Me-C). In certain embodiments, each cytosine is a 5-methylcytosine. In certain embodiments, the modified base is a 5-methyluracil (5′-Me-U). In certain embodiments, each uracil is a 5-methyluracil.

Any modified nucleotide or analogue that may benefit the stability or affinity can be made without departing from the spirit and scope of the present invention. Several examples of such chemical modifications are same as summarized above.

### 5. PHARMACEUTICAL COMPOSITION

In some embodiments, the present invention also provides pharmaceutical formulations comprising the asdDNA of the present invention, or a pharmaceutically acceptable derivative thereof and at least one pharmaceutically acceptable excipient or carrier. As used herein, “pharmaceutically acceptable excipient” or “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in “Remington: The Science and Practice of Pharmacy, Twentieth Edition,” Lippincott Williams & Wilkins, Philadelphia, PA., which is incorporated herein by reference. Examples of such carriers or diluents include, but are not limited to, water, saline, Ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the asdDNA molecule, use thereof in the compositions is contemplated.

Examples of the pharmaceutically acceptable carrier that can be used with the molecule of the invention include but are not limited to: a pharmaceutical carrier, a positive-charge carrier, a liposome, a lipid nanoparticle, a protein carrier, a hydrophobic moiety or molecule, a cationic moiety or molecule, GalNAc, a polysaccharide a polymer, a nanoparticle, a nanoemulsion, a cholesterol, a lipid, a lipophilic compound or moiety, and a lipoid.

In a certain embodiment, the present invention provides a method of treatment comprising administering a therapeutically effective amount of the pharmaceutical composition to a subject in need thereof. In an embodiment, the pharmaceutical composition is administered via a route selected from the group of: intravenous injection (iv), subcutaneous injection (sc), per os (po), intramuscular (im) injection, oral administration, inhalation, topical, intrathecal, and other regional administrations. In another embodiment, the therapeutically effective amount is 1 ng to 1 g per day, 100 ng to 1 g per day, or 1 μg to 1000 mg per day.

Methods for formulation are disclosed in PCT International Application PCT/US02/24262 (WO03/01 1224), U.S. Patent Application Publication No. 2003/0091639 and U.S. Patent Application Publication No. 2004/0071775, each of which is incorporated by reference herein.

An asdDNA molecule of the present invention is administered in a suitable dosage form prepared by combining a therapeutically effective amount (e.g., an efficacious level sufficient to achieve the desired therapeutic effect through inhibition of tumor growth, killing of tumor cells, treatment or prevention of cell proliferative disorders, etc.) of the asdDNA molecule of the present invention (as an active ingredient) with standard pharmaceutical carriers or diluents according to conventional procedures (i.e., by producing a pharmaceutical composition of the invention).

These procedures may involve mixing, granulating, and compressing or dissolving the ingredients as appropriate to attain the desired preparation. In another embodiment, a therapeutically effective amount of asdDNA molecules is administered in a suitable dosage form without standard pharmaceutical carriers or diluents. In some embodiments, a therapeutically effective amount of the duplex molecule of the invention is administered in a suitable dosage form. Pharmaceutically acceptable carriers include solid carriers such as lactose, terra alba, sucrose, talc, gelatin, agar, pectin, acacia, magnesium stearate, stearic acid and the like. Exemplary liquid carriers include syrup, peanut oil, olive oil, water and the like. Similarly, the carrier or diluent may include time-delay material known in the art, such as glyceryl monostearate or glyceryl distearate, alone or with a wax, eihylcellulose, hydroxypropylmethylcellulose, methylmethacrylate or the like. Other fillers, excipients, flavorants, and other additives such as are known in the art may also be included in a pharmaceutical composition according to this invention.

The pharmaceutical compositions of the present invention may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers comprising excipients and/or auxiliaries which facilitate processing of the sense oligonucleotide and the antisense oligonucleotide into preparations that can be used pharmaceutically. Of course, the appropriate formulation is dependent upon the route of administration chosen.

The composition, compound, combination or the pharmaceutical composition of the invention can be administered to a subject in many of the well-known methods currently used for chemotherapeutic treatment. For example, for treatment of cancers, the asdDNA molecule of the invention may be injected directly into tumors, injected into the blood stream or body cavities or taken orally or applied through the skin with patches. For treatment of psoriatic conditions, systemic administration (e.g., oral administration), or topical administration to affected areas of the skin, are preferred routes of administration. The dose chosen should be sufficient to constitute effective treatment but not as high as to cause unacceptable side effects. The state of the disease condition (e.g., cancer, psoriasis, and the like) and the health of the patient should be closely monitored during and for a reasonable period after treatment.

### 6. UTILITY

### 6.1 Method of Use

The present invention provides a method of modulating gene expression or function in a cell or an organism. The cell may be a eukaryotic cell, e.g., a mammalian cell. The method comprises the steps of contacting said cell or organism with the asdDNA molecule disclosed herein, under conditions wherein selective gene silencing can occur, and mediating a selective gene silencing effected by the asdDNA molecule towards a target nucleic acid having a sequence portion substantially complementary to the antisense strand. The target nucleic acid may be an RNA such as a mRNA or non-coding RNA where such RNA either encodes a protein or regulates a part of a biological pathway implicated in a disease.

In an embodiment, the contacting step comprises the step of introducing asdDNA molecule into a target cell in culture or in an organism in which the selective gene silencing can occur. In a further embodiment, the introducing step comprises a mixing, transfection, lipofection, infection, electroporation, or other delivery technologies. In another embodiment, the introducing step comprises using a pharmaceutically acceptable excipient, carrier, or diluent selected from the group of a pharmaceutical carrier, a positive-charge carrier, a liposome, a lipid nanoparticle, a protein carrier, a polymer, a nanoparticle, a nanoemulsion, a lipid, N-Acetyl-Galactosamine (GalNAc), a lipophilic compound or moiety and a lipoid to be administered via iv, sc, intrathecal, po, inhalation, topical or other clinically acceptable administration methods.

In an embodiment, the silencing method is used for determining the function or utility of a gene in a cell or an organism.

In an embodiment, the gene or RNA targeted by the composition of the invention is associated with or implicated in a disease, e.g., a human disease or an animal disease, a pathological condition, or an undesirable condition. In a further embodiment, the target gene or RNA is that of a pathogenic microorganism. In an even further embodiment, the target gene or RNA is of a viral origin. In another embodiment, the target gene or RNA is tumor-associated.

In an alternative embodiment, the gene or RNA targeted by the composition of the invention is a gene or a RNA associated with, or more specifically, implicated with cancer, autoimmune disease, inflammatory diseases, degenerative diseases, infectious diseases, proliferative diseases, metabolic diseases, immune-mediated disorders, allergic diseases, dermatological diseases, malignant diseases, gastrointestinal disorders, hepatic disorders, respiratory disorders, cardiovascular disorders, dermatological disorders, renal disorders, rheumatoid disorders, neurological disorders, psychiatric disorders, endocrine disorders, or aging-related disorders or diseases.

### 6.2 Treatment Method

The present invention also provides a method of treating or preventing various diseases or conditions, including those summarized for ASO and siRNAs (Czech, 2006; de Fougerolles et al., 2007; Dykxhoorn et al., 2003; Kim and Rossi, 2007; Mack, 2007; Crooke S T et al., 2018; Setten R L et al., 2019; Roberts T O et al., 2020). The method comprises administering an effective amount of the asdDNA molecule to a subject in need thereof under conditions wherein desired gene inhibition described in the section immediately above can occur.

In an exemplary embodiment, a pharmaceutical composition having the asdDNA molecule and a pharmaceutically acceptable excipient, carrier, or diluent is administered to a patient in need thereof for treating or preventing a disease or an undesirable condition in a therapeutically effective amount.

In some embodiments, the present invention can be used as a cancer therapy or to prevent cancer. The composition of the asdDNA can be used to silence or knock down genes involved with cell proliferation disorders or a malignant disease. Examples of these genes are k-Ras, β-catenin, Stat3. These oncogenes are active and relevant in a large number of human cancer.

The novel composition of the invention can also be used to treat or prevent ocular disease, (e.g., age-related macular degeneration (AMD) and diabetic retinopathy (DR)); infectious diseases (e.g., HIV/AIDS, hepatitis B virus (HBV), hepatitis C virus (HCV), human papillomavirus (HPV), herpes simplex virus (HSV), RCV, cytomegalvirus (CMV), dengue fever, west Nile virus); respiratory disease (e.g., respiratory syncytial virus (RSC), asthma, cystic fibrosis); neurological diseases (e.g., Huntingdon's disease (HD), amyotrophic lateral sclerosis (ALS), spinal cord injury, Parkinson's disease, Alzheimer's disease, pain); cardiovascular diseases; metabolic disorders (e.g., hyperlipidemia, hypercholesterolemia, and diabetes); genetic disorders; and inflammatory conditions (e.g., inflammatory bowel disease (IBD), arthritis, rheumatoid disease, autoimmune disorders), dermatological diseases.

In an alternative embodiment, the administration method is a route selected from the group of intravenous injection (iv), subcutaneous injection (sc), per os (po), intrathecal, inhalation, topical, and regional administration.

### EXAMPLES

Examples are provided below to further illustrate different features of the present invention. The examples also illustrate useful methodology for practicing the invention. These examples do not limit the claimed invention.

### Methods and Materials

### Cell Culture

DLD1 cell was purchased from ATCC. Cells were grown in Dulbecco's modified Eagle's medium (DMEM) supplemented with 10% inactivated fetal bovine serum (FBS).

HepaRG cells were grown in William's Medium supplemented with 10% FBS, 10 mg/ml Hydrcortisone, and 4 mg/ml human recombinant insulin.

Transfection of asdDNAs to DLD1 Cells or HepaRG Cells

24 hours before transfection, the DLD1 or HepaRG cells were seeded to 6-well plates (1×105 cells/2 mL/well). The asymmetric sdDNAs were transfected by Lipofectamine® RNAiMAX (Thermo Fisher, USA) at 100 pM, 200 pM, 1 nM, 3 nM, 10 nM, 30 nM or 100 nM final concentrations as described the manufacture methods, briefly asymmetric sdDNAs and RNAiMAX were incubate for 20 minutes in serum free OPTI-MEM (Thermo Fisher), then added to the cell with culture medium.

### Quantitative PCR

Cells transfected with the indicated asymmetric sdDNAs were harvested at 48 hours after transfection. RNA was isolated with TRIZOL, and qRT-PCR performed using TaqMan one-step RT-PCR reagents and CTNNB1 assays (Thermo Fisher) for β-catenin mRNA detection; APOCIII assay for APOCIII mRNA detection; APOB assay for APOB mRNA detection; TTR assay for TTR mRNA detection; STAT3 assay for STAT3 mRNA detection and the gene GAPDH mRNA levels were used as internal control.

### Target Sequences

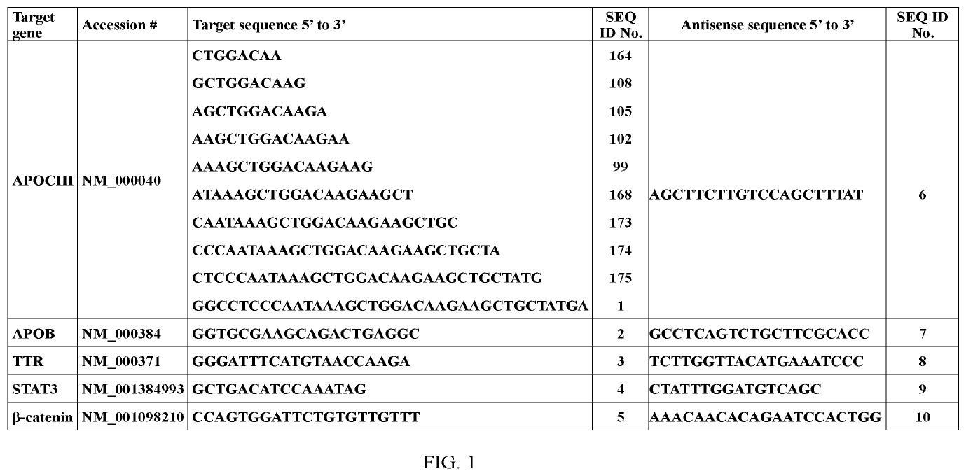

To investigate the gene silencing effects of the asdDNA disclosed in the present invention, asdDNAs were designed and made to target different genes. The target genes, target sequences designed and used in below examples are shown in FIG. 1, and exemplary sequences of corresponding antisense strand of asdDNAs, ASO or siRNA are also shown in FIG. 1.

### Example 1: Structure-Activity Relationship (SAR) on asdDNA with ISR in Both AS and SS

Structures and sequences of asymmetric sdDNAs with ISR in both AS (antisense strand) and SS (sense strand) or only in AS were designed and used, and are listed in FIG. 2A and FIG. 2B.

All the designed asdDNAs (sdDNA a1-a33) targeting APOCIII were transfected to the HepaRG cells. Relative mRNA level of APOCIII was detected after transfecting the sdDNA a1-a33 molecules to HepaRG cells at 100 pM. The gene silencing results are shown in FIG. 2C, which suggest that all the designed asdDNAs have highly potent gene silencing activity at very low concentrations (pico molar level).

In FIG. 2A, all Letters “D” in the illustrated structures represent DNA residues or deoxyribonucleotide monomers; all Letters “R” in the illustrated structures represent RNA residues or ribonucleotide monomers; all “*” in the illustrated structures represent PS (phosphorothioate internucleoside linkage).

In FIG. 2B, all lowercase Letters “a, c, g, t” in the sequences represent DNA residues; all uppercase Letters “A, C, G, U” in the sequences represent 2′-MOE modified RNA residues, wherein all “U” is 5-Methyl Uridine 2′-MOE RNA residues; wherein all “C” and “c” are 5-Me-C; all “*” in the sequences represent PS (phosphorothioate internucleoside linkage).

### Example 2: SAR on asdDNAs with ISR Disposed Exclusively in AS and PS-Modified DNA SS of Various Positions and Lengths

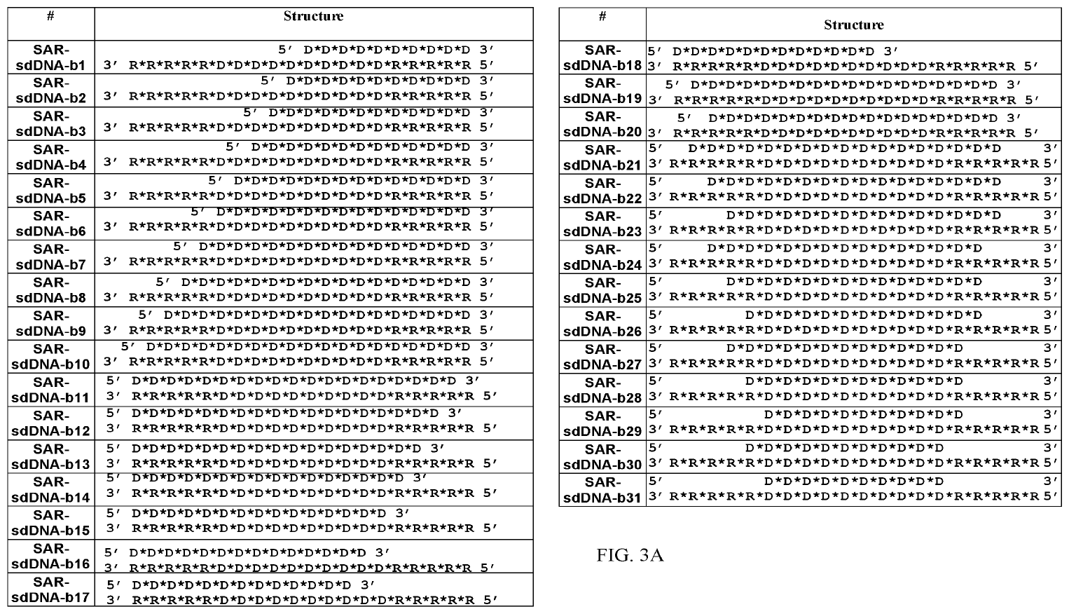

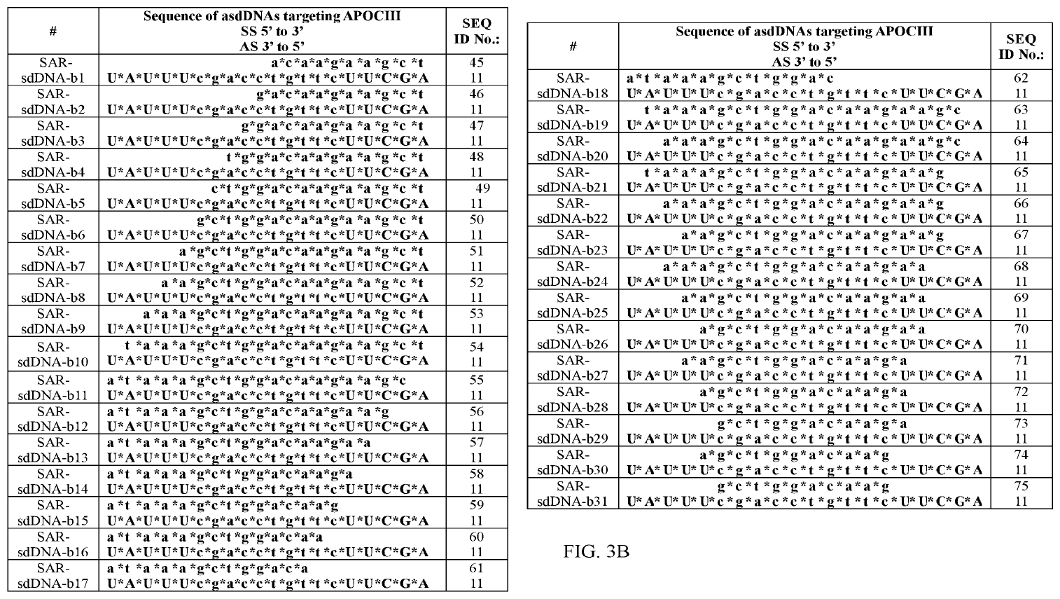

FIG. 3A shows various structures of a series of embodiments of asdDNA where ISRs are found exclusively in the AS. By holding the ISR-containing antisense strand (AS) constant and changing the position and length of the sense strand (SS) made up exclusively of PS-modified DNA (sdDNA b1-b31), the gene-silencing effects of such structural variations were tested. Specifically, asdDNA b1-b31 were designed to target the APOCIII gene (structures and sequences of asdDNA b1-b31 shown in FIG. 3B). Gene silencing activities of these asdDNAs were tested in HepaRG cells (FIG. 3C).

In FIG. 3A, all Letters “D”, “R” and “*” in the illustrated structures represent the same as in FIG. 2A. In FIG. 3B, all lowercase Letters “a, c, g, t”, all uppercase Letters “A, C, G, U” and “*” in the sequences represent the same as in FIG. 2B.

The results suggest that all the designed asdDNAs have highly potent gene silencing activity at very low concentrations (pico molar level), same conclusion as in Example 1.

### Example 3: SAR on asdDNA with ISR Disposed Exclusively in AS and Non-modified DNA SS of Various Position

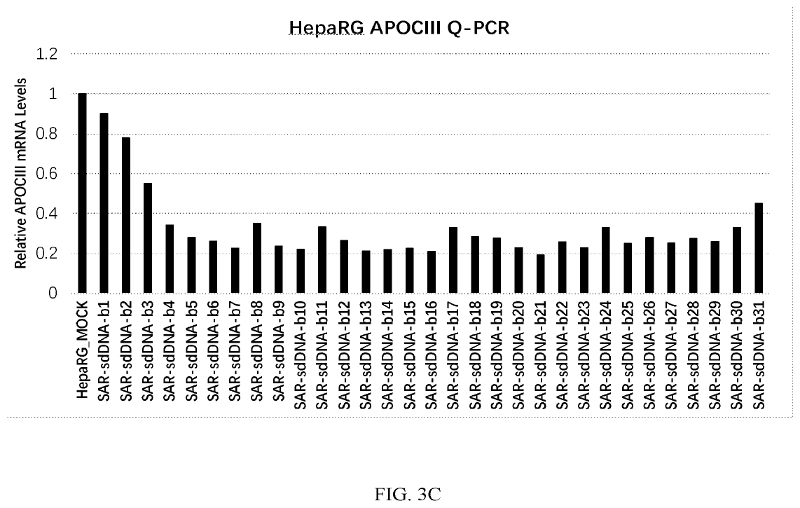

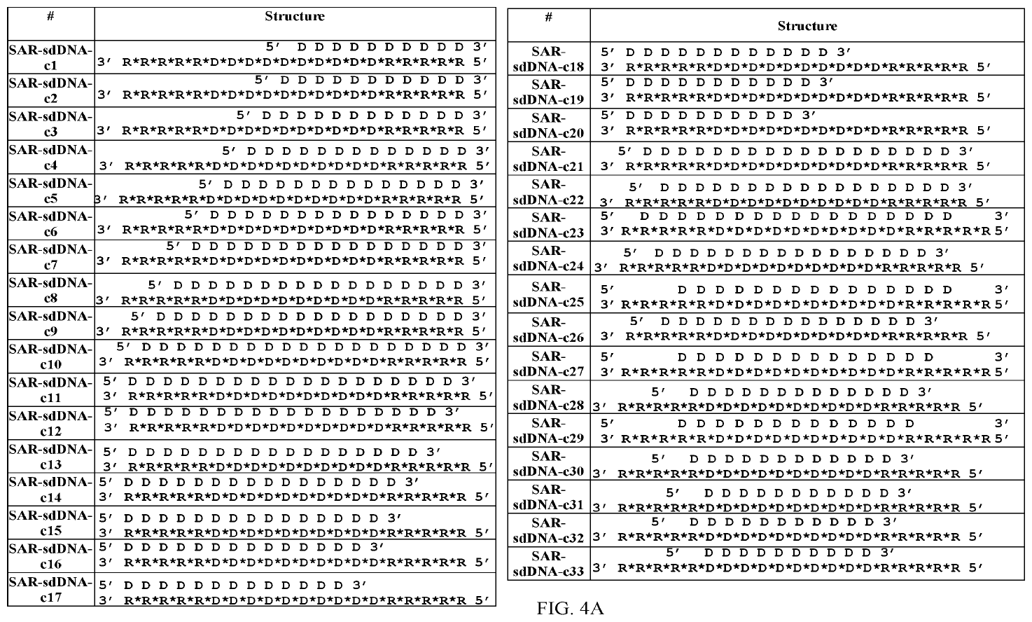

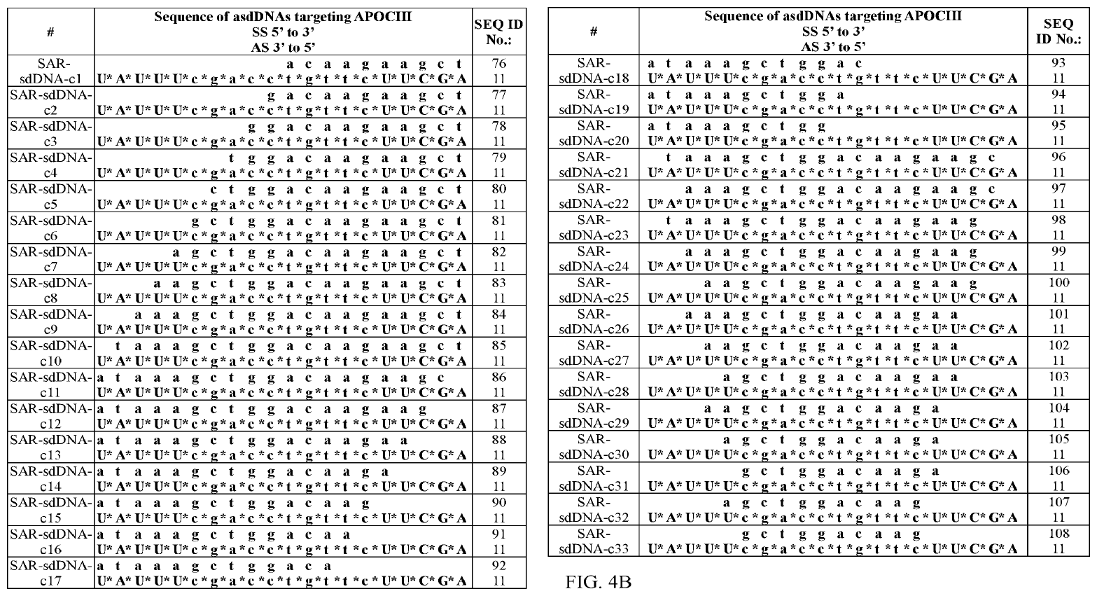

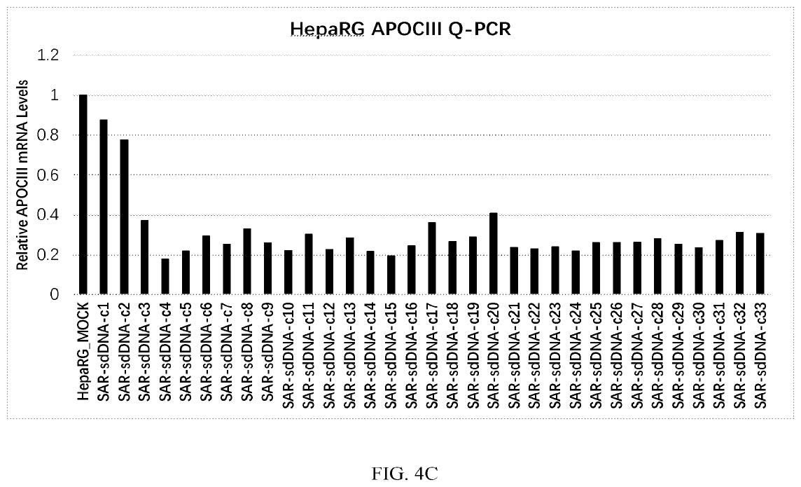

FIG. 4A shows different structural designs of another series of embodiments of asdDNA with ISR disposed exclusively in AS. In these asdDNAs, the AS was kept constant and the SS made up of pure natural DNA monomers is used. Various positions and lengths of SS were designed (sdDNA c1-c31) (structure and sequence shown in FIG. 4B). Gene-silencing effects of these asdDNA c1-c31 targeting APOCIII were tested in HepaRG cells (FIG. 4C).

In FIG. 4A, all Letters “D”, “R” and “*” in the illustrated structures represent the same as in FIG. 2A. In FIG. 4B, all lowercase Letters “a, c, g, t”, all uppercase Letters “A, C, G, U” and “*” in the sequences represent the same as in FIG. 2B.

The results suggest that all the designed asdDNAs have highly potent gene silencing activity at very low concentrations (pico molar level), same conclusion as in Examples 1 and 2.

### Example 4: SAR on asdDNA with ISRs Comprising Various Number of Ribonucleotide monomers Disposed at Various Positions in AS

FIG. 5A shows different structural designs of a further series of asdDNAs. In these asdDNAs, the SS was kept constant while changing ribonucleotide monomers of ISR in the antisense strand (sdDNA d1-d24) (structures and sequences shown in FIG. 5A). The single-stranded antisense oligonucleotide with the identical structure and sequence as the antisense strand of asdDNA d1-d20 are also designed as corresponding ASO of each asdDNA. Gene silencing activities of the asdDNA d1-d24 and each corresponding ASO designed to target APOCIII were tested in HepaRG Cells (the comparison results are shown in FIG. 5B).

In FIG. 5A, all Letters “D”, “R”, lowercase Letters “a, c, g, t”, uppercase Letters “A, C, G, U” and “*” in the illustrated structures and sequences represent the same as in FIG. 2A and FIG. 2B.

The results suggest that all designed asdDNAs with at least one ISR in AS have highly potent gene silencing activity at very low concentrations (pico molar level) and are significant more potent as well as more efficacious than the corresponding ASO.

### Example 5: SAR on asdDNA with ISRs Disposed at Various Positions in AS while Keeping the Total Number of Ribonucleotide Monomers in AS Constant

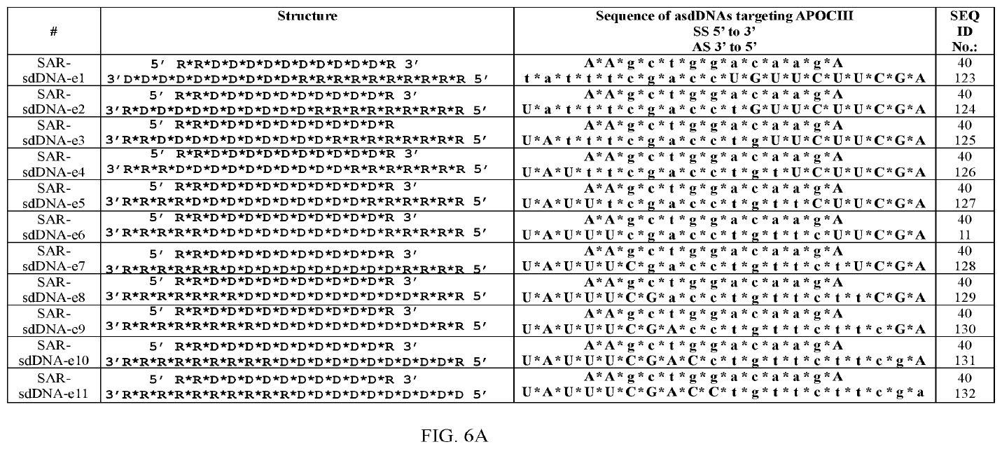

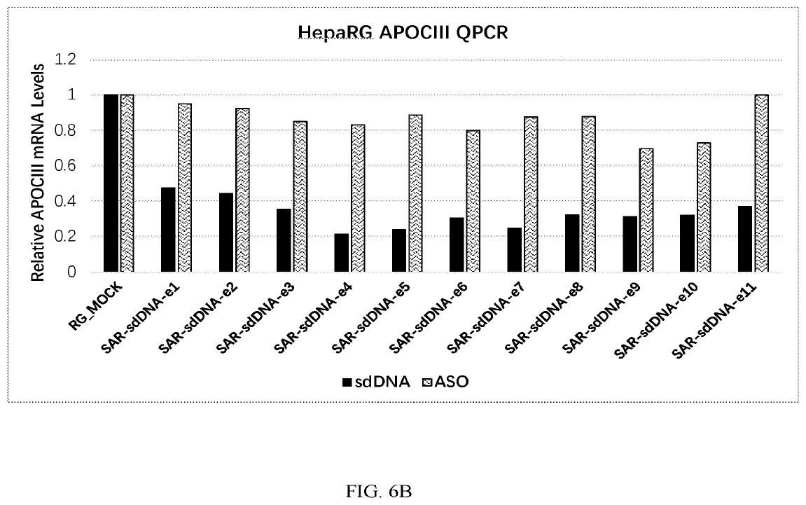

FIG. 6A shows different structural designs of yet another series of asdDNA. In these asdDNAs, the sense strand was kept constant while changing the positions of ISR(s), comprising of a fixed total number of ribonucleotide monomers, in the antisense strand (sdDNA e1-e11, structures and sequences in FIG. 6A). The single-stranded antisense oligonucleotide with the same structure and sequence as the antisense strand of sdDNA e1-e11 are also designed as corresponding ASO of each asdDNA. Gene silencing activities of asdDNA e1-e11 and each corresponding ASO designed to target APOCIII gene were tested in HepaRG Cells (the comparison results are shown in FIG. 6B).

In FIG. 6A, all Letters “D”, “R”, lowercase Letters “a, c, g, t”, uppercase Letters “A, C, G, U” and “*” in the illustrated structures and sequences represent the same as in FIG. 2A and FIG. 2B.

The results suggest that all designed asdDNAs have highly potent gene silencing activity at very low concentrations (pico molar level) and are significant more potent as well as more efficacious than the corresponding ASO, same conclusion as in Example 4.

### Example 6: SAR on asdDNA with Various Lengths of AS

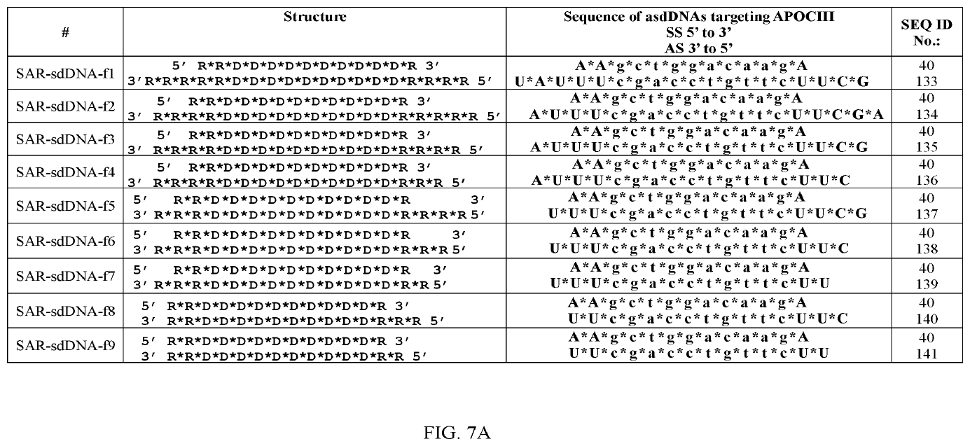

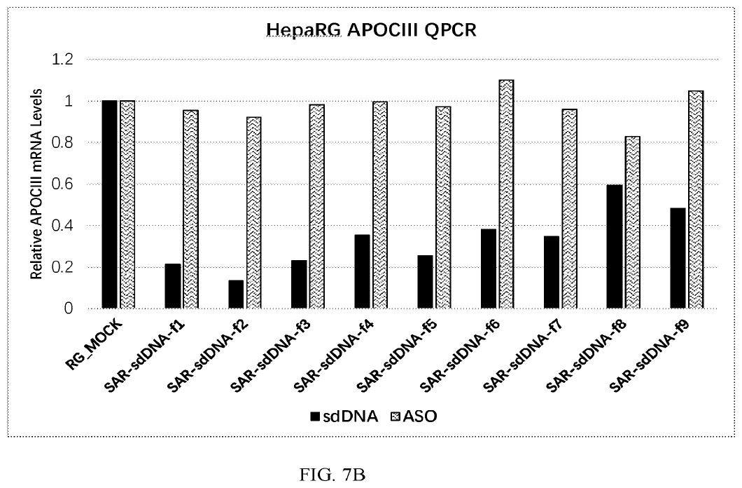

FIG. 7A shows different structural designs of a further series of asdDNAs. In these asdDNAs, the sense strand was kept constant while changing the length of the antisense strand (sdDNA f1-f9, structures and sequences shown in FIG. 7A). The single-stranded antisense oligonucleotide with the same structure and sequence as the antisense strand of asdDNA f1-f9 are also designed as corresponding ASO of each asdDNA. Gene silencing activities of sdDNA f1-f9 and each corresponding ASO designed to target APOCIII were tested in HepaRG Cells (the comparison results are shown in FIG. 7B).

In FIG. 7A, all Letters “D”, “R”, lowercase Letters “a, c, g, t”, uppercase Letters “A, C, G, U” and “*” in the illustrated structures and sequences represent the same as in FIG. 2A and FIG. 2B.

The results suggest that all designed asdDNAs have highly potent gene silencing activity at very low concentrations (pico molar level) and are significant more potent as well as more efficacious than the corresponding ASO, same conclusion as in Examples 4 and 5.

### Example 7: SAR on asdDNAs with Various Lengths of AS and SS

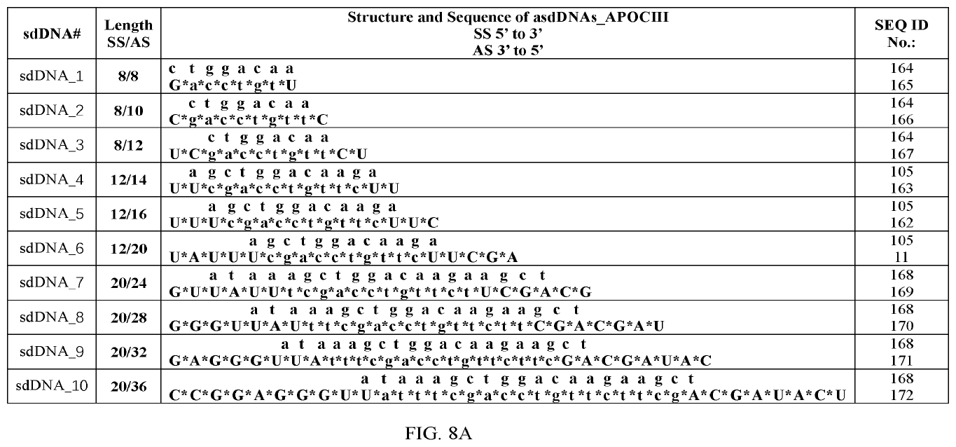

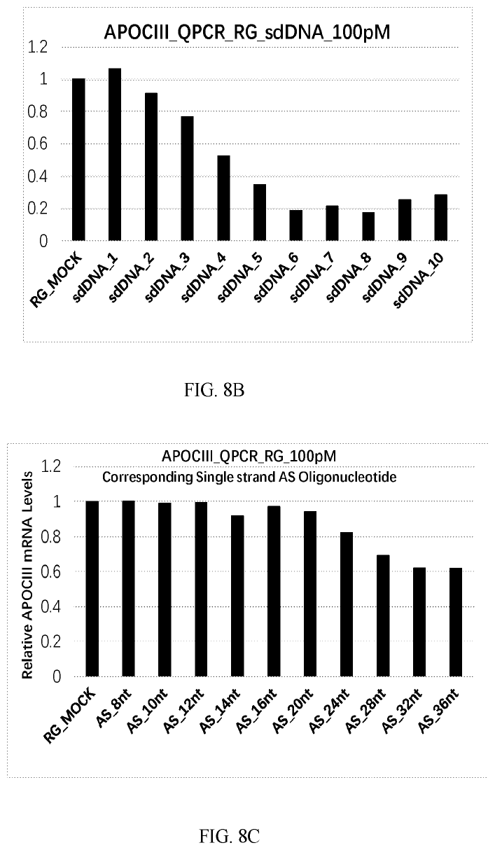

FIG. 8A shows different structural designs of a further series of asdDNAs. In these asdDNAs, various length of the antisense strand and the sense strand for targeting the APOCIII gene were designed (sdDNA_1-10 structures and sequences shown in FIG. 8A). The single-stranded antisense oligonucleotide with the same structure and sequence as the antisense strand of asdDNA_1-10 are also designed as corresponding single-stranded AS of each asdDNA. Gene silencing activities of asdDNA_1-10 and each corresponding single-stranded AS designed to target APOCIII were tested in HepaRG Cells (results of asdDNAs are shown in FIG. 8B, corresponding ASO are shown in FIG. 8C).

In FIG. 8A, all lowercase Letters “a, c, g, t”, uppercase Letters “A, C, G, U” and “*” in the illustrated sequences represent the same as in FIG. 2B.

The results suggest that all designed asdDNAs have highly potentgene silencing activity at very low concentrations (pico molar level) and are significant more potent as well as more efficacious than the corresponding single-stranded AS, similar conclusion as in Examples 4-6. Corresponding single-stranded AS here shows generally very low activities at pico molar level, but can show gene silencing activity at nano molar level (about 10 nM-30 nM) same with known development of well-known ASO technology. It is also a surprising discovery here that single-stranded AS Oligonucleotide with much longer length than typical ASO (generally has length of 16 nt to 20 nt) shows more potency of gene silencing activity than the typical ASO. However, the asdDNA of this invention always possesses much more potency and efficacy than the corresponding single-stranded AS Oligonucleotide, including known much optimized ASO.

### Example 8: SAR on asdDNAs with Various Lengths of AS and SS

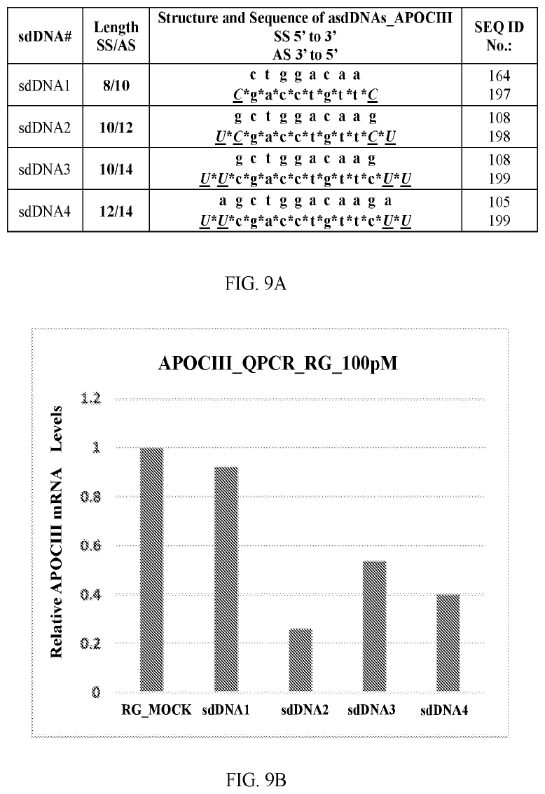

FIG. 9A shows different structural designs of a further series of asdDNAs. In these asdDNAs, various length of the antisense strand and the sense strand for targeting the APOCIII gene were designed (sdDNA1-4, structures and sequences shown in FIG. 9A). In FIG. 9A, all lowercase Letters “a, c, g, t” and “*” in the illustrated sequences represent the same as in FIG. 2B, all uppercase Letters underlined “A, C, G, U” in the illustrated sequences represent LNA modified RNA residues, wherein all “U” is 5-Methyl Uridine LNA RNA residues and all “C” arc 5-Me-C LNA RNA residues.

Gene silencing activities of asdDNA_1-10 and each corresponding single-stranded ASO designed to target APOCIII were tested in HepaRG Cells (results of asdDNAs are shown in FIG. 9B). The results suggest that all designed asdDNAs have highly potent gene silencing activity at very low concentrations (pico molar level), similar conclusion as in Examples 1-3.

### Example 9: SAR on asdDNAs with Various Lengths of SS

FIG. 10A shows different structural designs of a further series of asdDNAs. In these asdDNAs, the antisense strand was kept constant with length of 32 nt while changing the length of the sense strand from 8 nt to 28 nt (asdDNA_1-8, structures and sequences shown in FIG. 10A). In FIG. 10A, all lowercase Letters “a, c, g, t”, uppercase Letters “A, C, G, U” and “*” in the illustrated sequences represent the same as in FIG. 2B. The single-stranded antisense oligonucleotide with the identical structure and sequence as the antisense strand of all asdDNAs, with length of 32 nt, also designed as corresponding single-stranded ASO for comparison. Gene silencing activities of asdDNA_1-8 and the corresponding single-stranded ASO designed to target APOCIII were tested in HepaRG Cells (results are shown in FIG. 10B).

The results suggest that all designed asdDNAs have highly potent gene silencing activity at very low concentrations (pico molar level) and are much more potent as well as much more efficacious than the corresponding single-stranded ASO, even when the ASO has a considerable length, longer than the known typical ASO technology (general has length of 16 nt to 20 nt), same conclusion as in Example 7.

### Example 10: SAR on asdDNAs with Various Lengths of SS

FIG. 11A shows different structural designs of a further series of asdDNAs. In these asdDNAs, the antisense strand was kept constant with length of 36 nt while changing the length of the sense strand from 8 nt to 32 nt (sdDNA_1-9, structures and sequences shown in FIG. 11A). In FIG. 11A, all lowercase Letters “a, c, g, t”, uppercase Letters “A, C, G, U” and “*” in the illustrated sequences represent the same as in FIG. 2B. The single-stranded antisense oligonucleotide with the same structure and sequence as the antisense strand of all asdDNAs, with length of 36 nt, also designed as corresponding single-stranded ASO for comparison. Gene silencing activities of asdDNA_1-9 and the corresponding single-stranded ASO designed to target APOCIII were tested in HepaRG Cells (results are shown in FIG. 11B).

The results suggest that all designed asdDNAs have highly potent gene silencing activity at very low concentrations (pico molar level) and are much more potent as well as more efficacious than the corresponding single-stranded ASO, including when the ASO is considerably longer than the known typical ASO technology (general has length of 16 nt to 20 nt), same conclusion as in Examples 7 and 9.

### Example 11: SAR on asdDNAs with Various Lengths of AS

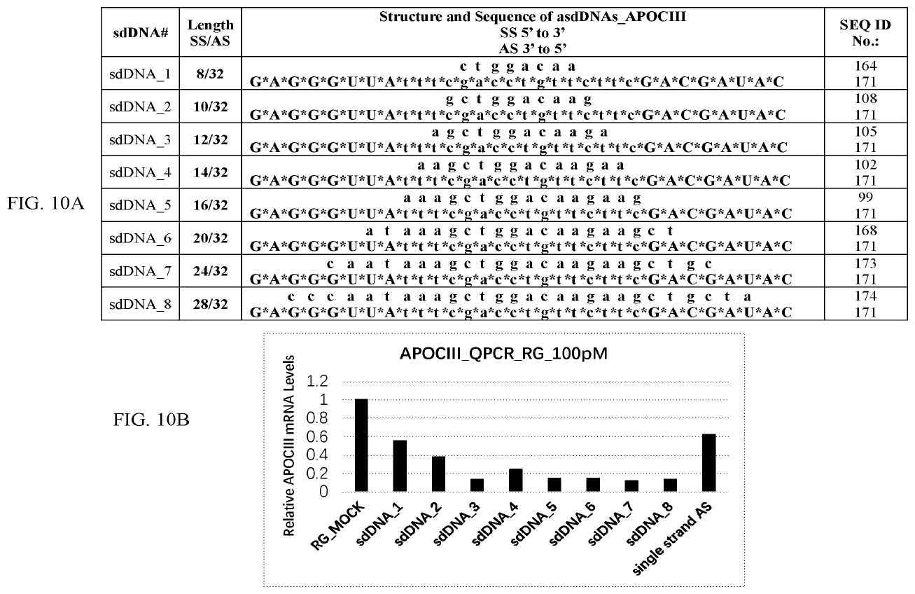

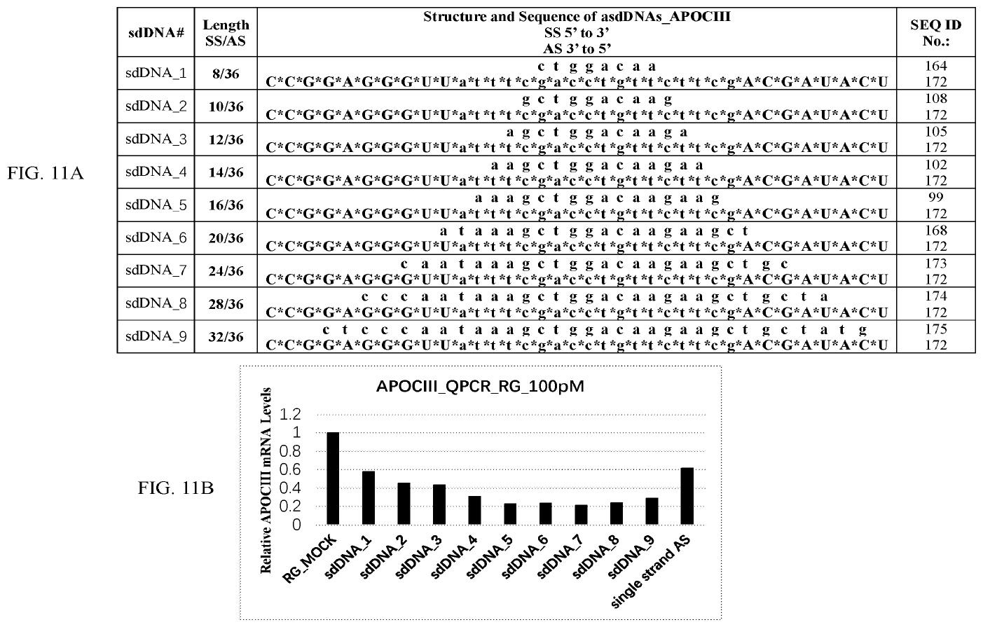

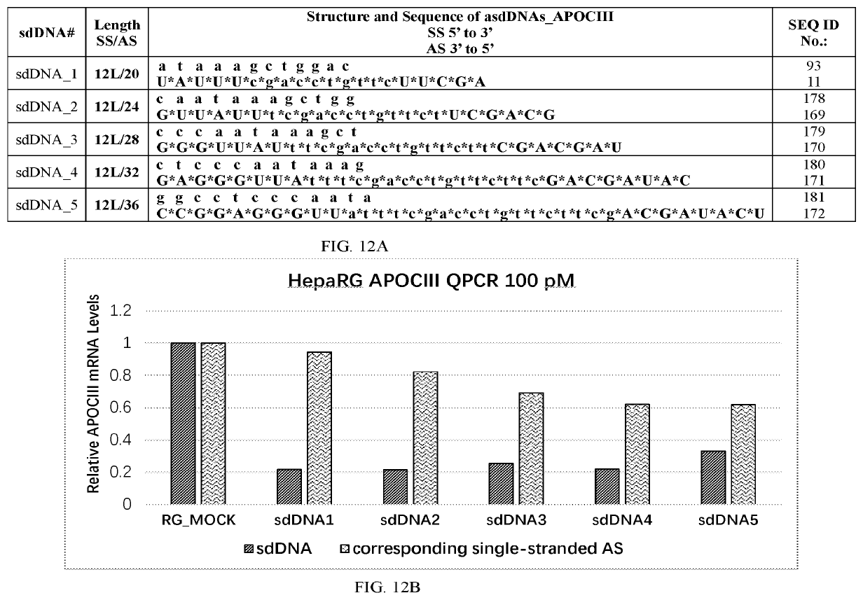

FIG. 12A shows different structural designs of a further series of asdDNAs. In these asdDNAs, the sense strand was kept constant with length of 12 nt while changing the length of the antisense strand from 20 nt to 36 nt (sdDNA_1-5, structures and sequences shown in FIG. 12A). In FIG. 12A, all lowercase Letters “a, c, g, t”, uppercase Letters “A, C, G, U” and “*” in the illustrated sequences represent the same as in FIG. 2B. The single-stranded antisense oligonucleotide with same structure and sequence of the antisense strand of each sdDNAs are also designed as corresponding single-stranded AS for comparison. Gene silencing activities of sdDNA_1-5 and each corresponding single-stranded AS designed to target APOCIII were tested in HepaRG Cells (results are shown in FIG. 12B).

The results suggest that all designed asdDNAs have highly potent gene silencing activity at very low concentrations (pico molar level) and are much more potent as well as much more efficacious than the corresponding single-stranded ASO, including corresponding single-stranded ASOs that was optimized with the most advanced state-of-art know hows, such as single-stranded ASO SEQ ID No.: 11, which is ISIS304801. The results suggest same conclusion as in Examples 7, 9 and 10.

### Example 12: SAR on asdDNA with Various Motif of ISR Disposed at Various Positions in AS

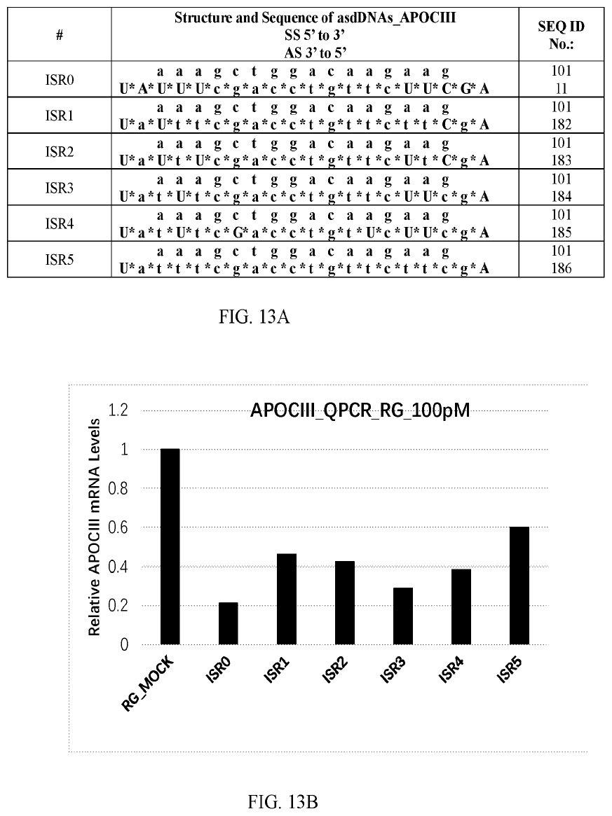

FIG. 13A shows different structural designs of a further series of asdDNAs. In these asdDNAs, the sense strand was kept constant while changing the positions and total number of ribonucleotide monomers of ISR(s) in the AS (ISR_0-5, structures and sequences shown in FIG. 13A). The various motif of ISR in the antisense strand in FIG. 13A shows that each ISR have a number of ribonucleotide monomers as low as 1 or 2 and each ISR is spaced apart with at least one intervening deoxyribonucleotide monomers. In FIG. 13A, all lowercase Letters “a, c, g, t”, uppercase Letters “A, C, G, U” and “*” in the illustrated sequences represent the same as in FIG. 2B. Gene silencing activities of ISR_0-5 designed to target APOCIII were tested in HepaRG Cells (results are shown in FIG. 13B).

All designed asdDNAs have highly potent gene silencing activity at very low concentrations (pico molar level). The results further suggest that the at least one ISR(s) in AS having various numbers of ribonucleotide monomers and disposed at any position in AS enables highly potent gene silencing activities of the asdDNAs provided in this invention.

### Example 13: SAR on asdDNAs with Mismatch in AS

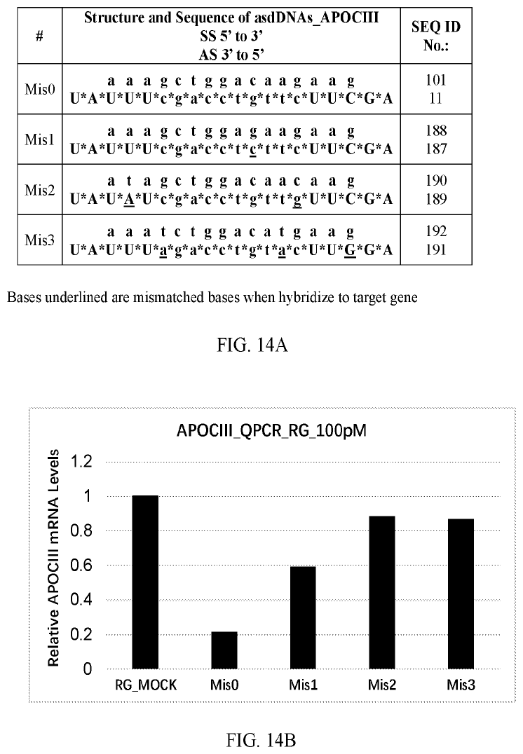

FIG. 14A shows different structural designs of a further series of asdDNAs. In these asdDNAs, the antisense strand was design to comprise at least one mismatch when hybridize to a target gene (Mis1-3, structures and sequences shown in FIG. 14A) and antisense strand has no mismatch (Mis0) was designed as comparison. In FIG. 14A, all lowercase Letters “a, c, g, t”, uppercase Letters “A, C, G, U” and “*” in the illustrated sequences are represent the same as in FIG. 2B. Gene silencing activities of Mis_0-3 designed to target APOCIII were tested in HepaRG Cells (results are shown in FIG. 14B).

All designed asdDNAs have highly potent gene silencing activity at very low concentrations (pico molar level). The results further suggest that the antisense strand of the asdDNA provided in this invention can tolerate at least three (at least 15% of the targeting region) mismatches while maintaining gene silencing activities of the asdDNA provided in this invention. Some mismatch or multiple mismatches in certain position in AS may reduce the gene silencing activity of the asdDNA.

### Example 14: SAR on asdDNA with Mismatch in SS

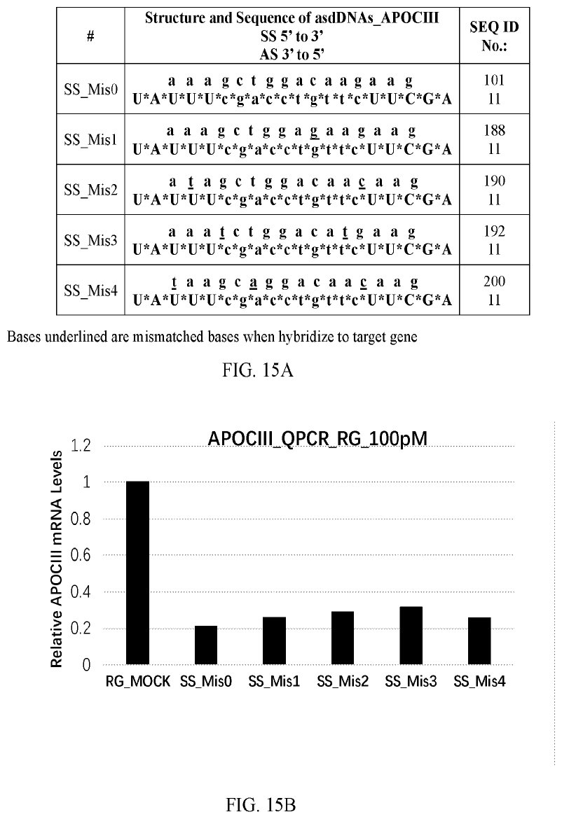

FIG. 15A shows different structural designs of a further series of asdDNAs. In these asdDNAs, the sense strand was design to comprise at least one mismatch when form double-stranded region with antisense strand (Mis1-4, structures and sequences shown in FIG. 15A) and sense strand has no mismatch (Mis0) was designed as comparison. In FIG. 15A, all lowercase Letters “a, c, g, t”, uppercase Letters “A, C, G, U” and “*” in the illustrated sequences represent the same as in FIG. 2B. Gene silencing activities of Mis_0-4 designed to target APOCIII were tested in HepaRG Cells (results are shown in FIG. 15B).

All designed asdDNAs have highly potent gene silencing activity at very low concentrations (pico molar level). The results further suggest that the sense strand of the asdDNA provided in this invention can tolerate three or more (at least 15% of the targeting region) mismatches while maintaining highly potent gene silencing activities of the asdDNA provided in this invention. Mismatches in sense strand can often help to reduce potential off-target effects.

### Example 15: Comparison Between asdDNA and siRNA

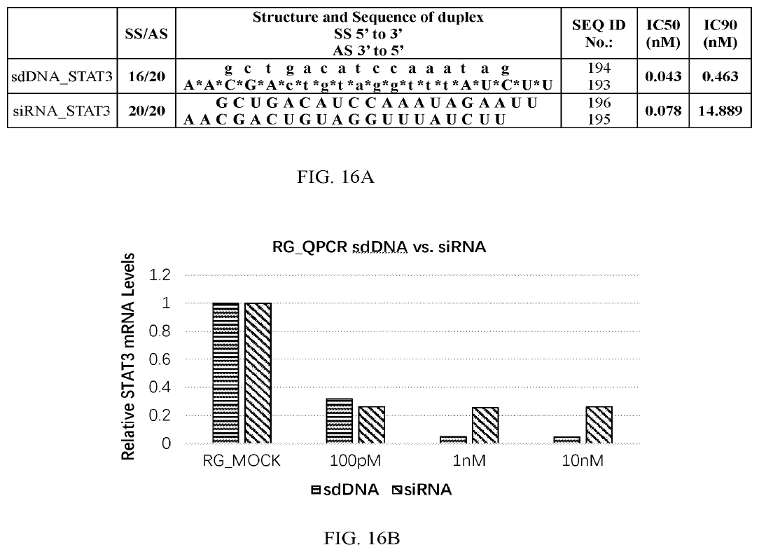

FIG. 16A shows sequence of an exemplary asdDNA of this invention and its corresponding siRNA for targeting the STAT3 gene. In FIG. 16A, all lowercase Letters “a, c, g, t”, uppercase Letters “A, C, G, U” and “*” in the illustrated sequence of asdDNA represent the same as in FIG. 2B, uppercase Letters “A, C, G, U” in the illustrated sequence of siRNA represent RNA residues. The corresponding siRNA has same nucleobase sequence with the antisense strand of the asdDNA. Comparison of gene silencing potency represented by IC₅₀ and IC₉₀ of the asdDNA and corresponding siRNA designed to target STAT3 are shown in FIG. 16A. FIG. 16B shows the comparison of gene silencing activities of the asdDNA and corresponding siRNA tested at 100 pM, 1 nM and 10 nM in HepaRG Cells.

The results suggest that the asdDNA provided in this invention, besides other aforementioned various pharmaceutical advantages than siRNA, may also have improved potency and efficacy of gene silencing activity than corresponding siRNA.

### Example 16: Gene Silencing Potency of asdDNAs Targeting APOCIII

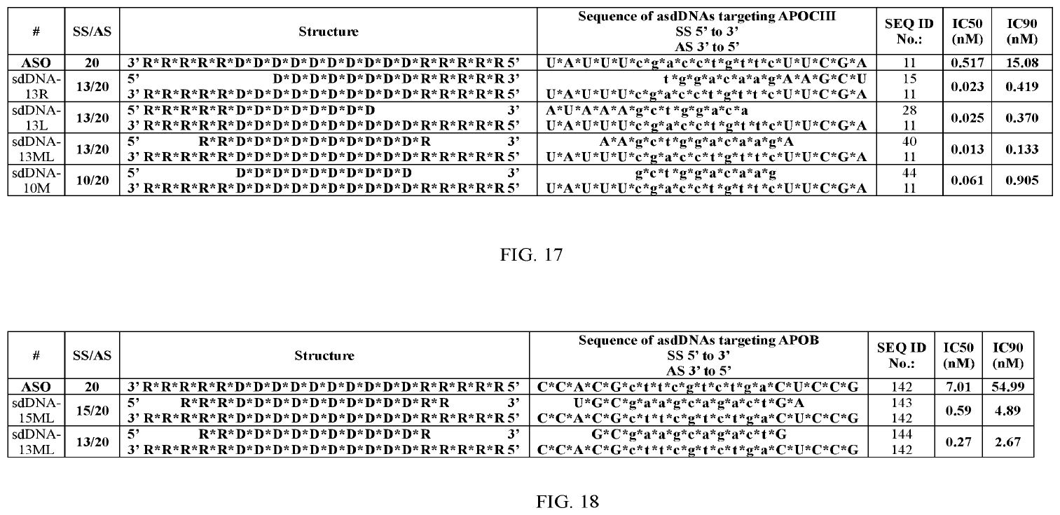

Gene silencing potency of exemplary asdDNAs targeting APOCIII and corresponding ASO were tested. FIG. 17 shows the structures, sequences and the values of IC₅₀ and IC₉₀) of the tested asdDNAs and corresponding ASO targeting APOCIII. In FIG. 17, all lowercase Letters “a, c, g, t”, uppercase Letters “A, C, G, U” and “*” in the illustrated sequence of asdDNA represent the same as in FIG. 2B.

### Example 17: Gene Silencing Potency of asdDNA Targeting APOB

Structures and sequences of exemplary asdDNAs targeting APOB and its corresponding ASO designed and used are shown in FIG. 18. In FIG. 18, all lowercase Letters “a, c, g, t”, uppercase Letters “A, C, G, U” and “*” in the illustrated sequence of asdDNA represent the same as in FIG. 2B.

Gene silencing potency of exemplary asdDNAs targeting APOB and corresponding ASO were tested. FIG. 18 shows the structures and the value of IC₅₀ and IC₉₀ of the asdDNAs and corresponding ASO targeting APOB.

### Example 18: Gene Silencing Potency of asdDNA Targeting TTR

Structures and sequences of exemplary asdDNAs targeting TTR and its corresponding ASO designed and used are shown in FIG. 19. In FIG. 19, all lowercase Letters “a, c, g, t”, uppercase Letters “A, C, G, U” and “*” in the illustrated sequence of asdDNA represent the same as in FIG. 2B.

Gene silencing potency of exemplary asdDNAs targeting TTR and corresponding ASO were tested. FIG. 19 shows the structures and the value of IC₅₀ and IC₉₀ of the asdDNAs and corresponding ASO targeting TTR.

### Example 19: Gene Silencing Potency of asdDNA Targeting STAT3

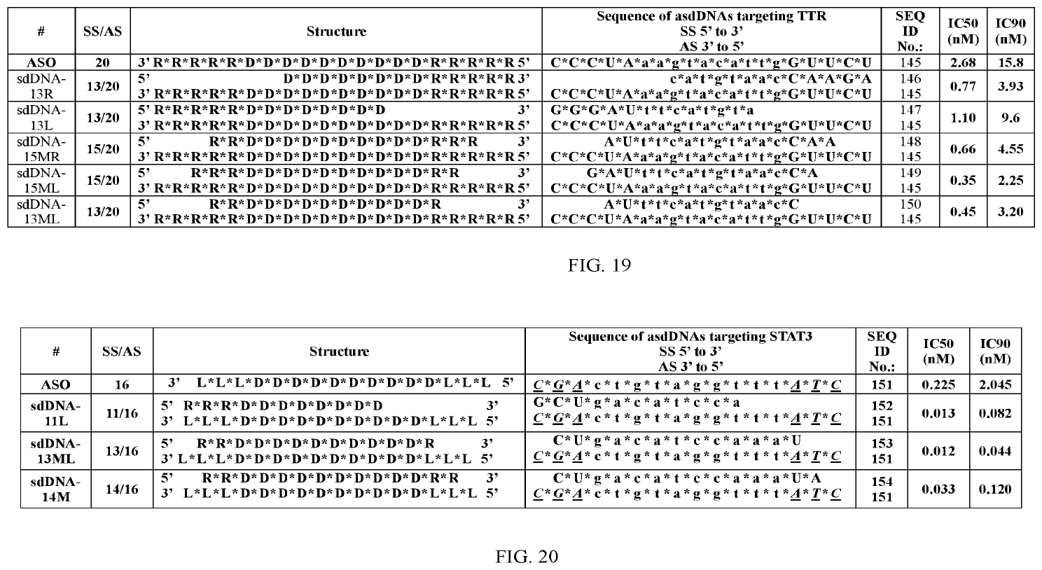

Structures and sequences of exemplary asdDNAs targeting STAT3 and its corresponding ASO designed and used are shown in FIG. 20. In FIG. 20, all lowercase Letters “a, c, g, t”, uppercase Letters underlined “A, C, G, U” and “*” in the illustrated sequence of asdDNA represent the same as in FIG. 9B.

Gene silencing potency of exemplary asdDNAs and corresponding ASO targeting STAT3 were tested. FIG. 20 shows the structures and the value of IC₅₀ and IC₉₀ of the asdDNAs and corresponding ASO targeting STAT3.

### Example 20: Gene Silencing Potency of asdDNA Targeting β-Catenin

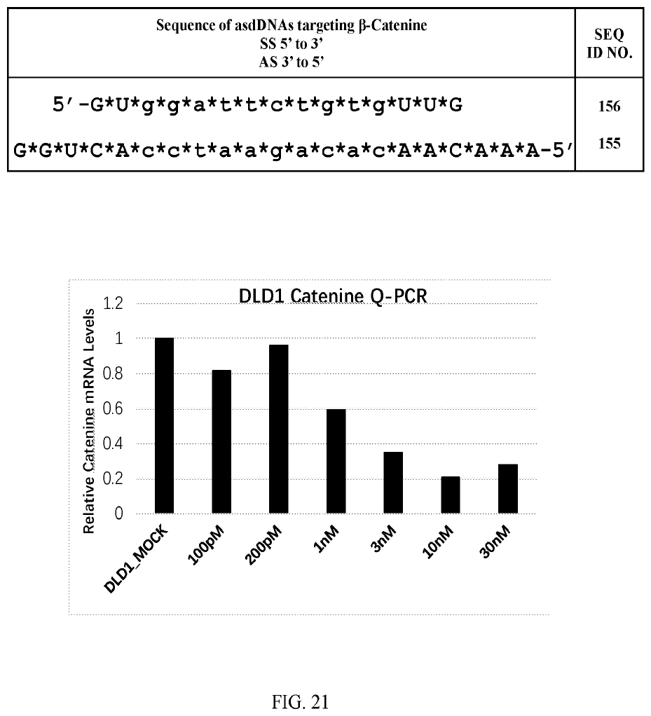

Structure and sequence of an asdDNA targeting ß-catenin designed and used is listed in FIG. 21. Gene silencing potency of the asdDNA targeting ß-catonin at 100 pM, 200 pM, 1 nM, 3 nM, 10 nM and 30 nM in DLD1 cells were tested. Results are shown in FIG. 21. In FIG. 21, all lowercase Letters “a, c, g, t”, uppercase Letters “A, C, G, U” and “*” in the illustrated sequence of asdDNA represent the same as in FIG. 2B.

These results in Examples 1-20 have strongly suggested that asdDNAs designed based on present invention can achieve great gene silencing potency for targeting different genes.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### Incorporation by Reference

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### REFERENCE

1. Elbashir S M, Harborth J, Lendeckel W, Yalcin A, Weber K, Tuschl T. Duplexes of 21-nucleotide RNAs mediate RNA interference in cultured mammalian cells. Nature. 2001 May 24: 411(6836): 494-8. doi: 10.1038/35078107. PMID: 11373684
2. Sun Xiangao, Rogoff Harry A, Li Chiang J. Asymmetric RNA duplexes mediate RNA interference in mammalian cells. Nat Biotechnol. 2008 December: 26(12): 1379-82. doi: 10.1038 nbt.1512. Epub 2008 Nov. 23. Erratum in: Nat Biotechnol. 2009 Feb. 27(2): 205. PMID: 19029911.
3. C. Frank Bennett and Eric E. Swayze, RNA Targeting Therapeutics: Molecular Mechanisms of Antisense Oligonucleotides as a Therapeutic Platform. Annu. Rev. Pharmacol. Toxicol. 2010. 50:259-93.
4. C. Frank Bennett. Therapeutic Antisense Oligonucleotides Are Coming of Age. Annu Rev Med. 2019 Jan. 27:70:307-321. doi: 10.1146 annurev-med-041217-010829. PMID: 30691367.
5. Setten R L, Rossi J J, Han S P. The current state and future directions of RNAi-based therapeutics. Nat Rev Drug Discov. 2019 Jun. 18(6): 421-446. doi: 10.1038 s41573-019-0017-4. Erratum in: Nat Rev Drug Discov. 2019 Mar. 18: Erratum in: Nat Rev Drug Discov. 2019 Apr. 24: PMID: 30846871.
6. Sibley C R, Seow Y, Wood M J. Novel RNA-based strategies for therapeutic gene silencing. Mol Ther. 2010 Mar. 18(3):466-76. doi: 10.1038 mt.2009.306. Epub 2010 Jan. 19. PMID: 20087319: PMCID: PMC2839433.
7. Grimm D. Asymmetry in siRNA design. Gene Ther. 2009 Jul. 16(7):827-9. doi: 10.1038 gt. 2009.45. Epub 2009 Apr. 30. PMID: 19404320.
8. Crooke S T, Witztum J L, Bennett C F, Baker B F. RNA-Targeted Therapeutics. Cell Metab. 2018 Apr. 3:27(4): 714-739. doi: 10.1016 j.cmet. 2018.03.004. Erratum in: Cell Metab. 2019 Feb. 5:29(2):501. PMID: 29617640.
9. Roberts T C, Langer R, Wood M J A. Advances in oligonucleotide drug delivery. Nat Rev Drug Discov. 2020 Oct. 19(10):673-694. doi: 10.1038 s41573-020-0075-7. Epub 2020 Aug. 11. PMID: 32782413: PMCID: PMC7419031.
10. Ryszard Kole, Adrian R. Krainer, Sidney Altman, RNA therapeutics: Beyond RNA interference and antisense oligonucleotids. Nat Rev Drug Discov. 2016. 11(2): 125-140.
11. Cy A. Stein, Daniela Castanotto, FDA-Approved Oligonucleotide Therapies in 2017. Molecular Therapy. 2017. Vol. 25 No 5 May 2017
12. Richard G. Lee, Jeff Crosby, Brenda F. Baker, Mark J. Graham, Rosanne M. Crooke, Antisense Technology: An Emerging Platform for Cardiovascular Disease Therapeutics. J. of Cardiovasc. Trans. Res. 2013. DOI 10.1007/s12265-013-9495-7
13. Zamecnik, P. C., & Stephenson, M. L. Inhibition of Rous sarcoma virus replication and cell transformation by a specific oligodeoxynucleotide. Proceedings of the National Academy of Sciences USA 75, 1978. 280-284.
14. Stanley T. Crooke, Molecular Mechanisms of Antisense Oligonucleotides. NUCLEIC ACID THERAPEUTICS. Volume 27, Number 2, 2017 Mary Ann Liebert, Inc. DOI: 10.1089/nat.2016.0656
15. Antisense Drug Technologies: Principles, Strategies, and Applications. 2. Crooke, S T., editor. CRC Press: Boca Raton, Florida: 2008
16. Fire, A., Xu, S., Montgomery, M. K., Kostas, S. A., Driver, S. E., and Mello, C. C. Potent and specific genetic interference by double-stranded RNA in Caenorhabditis elegans. Nature. 1998. 391, 806-811
17. de Fougerolles A, Vornlocher H P, Maraganore J, Lieberman J. Interfering with disease: a progress report on siRNA-based therapeutics. Nature Rev Drug Discov. 2007: 6:443-453. [PubMed: 17541417]
18. Jackson A L, Bartz S R, Schelter J M, Kobayashi S V, Burchard J, et al. 2003. Expression profiling reveals off-target gene regulation by RNAi. Nat. Biotechnol.21:635-37
19. Lin X, Ruan X, Anderson M G, McDowell J A, Kroeger P, et al. 2005. siRNA-mediated off-target gene silencing triggered by a 7nt complementation. Nucleic Acids Res.33:4527-35
20. Kwoh J T. 2008. An overview of the clinical safety experience of first- and second-generation antisense oligonucleotides. See Ref. 9, pp. 365-99
21. Henry S P, Kim T-W, Kramer-Strickland K, Zanardi T A, Fey R A, Levin A A. 2008. Toxicological properties of 2′-O-methoxyethyl chimeric antisense inhibitors in animals and man. See Ref. 9, pp. 327-63
22. Geary, R S.; Yu, R Z.; Levin, A A. Antisense Drug Technologies: Principles, Strategies, and Applications. See Ref. 9, pp. 183-217
23. Iwamoto N, Butler D, Syrzikapa N, Mohapatra S., Verdine G L. Control of phosphorothioate stereochemistry substantially increases the efficacy of antisense oligonucleotides Nat Biotechnol 35(9):845-851, 2017 doi: 10.1038/nbt.3948. Epub 2017 Aug. 21.

1. An asymmetric short duplex DNA (asdDNA) molecule comprising a first strand and a second strand,
wherein the second strand is shorter than the first strand;
wherein the first strand is substantially complementary to a targeted segment of a targeted RNA through at least one targeting region;
wherein the second strand is substantially complementary to the first strand, and forms at least one double-stranded region with the first strand; and
wherein the asdDNA molecule comprises at least one interspersed segment of ribonucleotide monomer(s) (ISR) that comprises at least one ribonucleotide monomer.

2. The asdDNA molecule of claim 1, wherein the first strand comprises at least one ISR.

3. The asdDNA molecule of claim 1, wherein the second strand comprises at least one ISR.

4. The asdDNA molecule of claim 1, wherein the first strand comprises at least one ISR and the second strand also comprises at least one ISR.

5. The asdDNA molecule of claim 2 or 4, wherein the at least one ISR is disposed in at least one targeting region of the first strand.

6. The asdDNA molecule of claim 5, wherein the total number of ribonucleotide monomers of all ISR(s) in the first strand is at least 2.

7. The asdDNA molecule of claim 3 or 4, wherein the at least one ISR is disposed in at least one double-stranded region of the second strand.

8. The asdDNA molecule of any one of claims 1 to 7, wherein the asdDNA molecule comprises at least two or more ISRs, and wherein each ISR, independently of each other, either consists of one ribonucleotide monomer, or comprises at least 2, 3, 4 or 5 contiguous ribonucleotide monomers.

9. The asdDNA molecule of any one of claims 1 to 7, wherein the at least one ISR comprises at least 2, 3, 4 or 5 contiguous ribonucleotide monomers.

10. The asdDNA molecule of any one of claims 1 to 9, wherein the first strand is at least 70%, 80%, 85%, 90%, 95% complementary or fully complementary to the targeted segment of the targeted RNA.

11. The asdDNA molecule of any one of claims 1 to 10, wherein the first strand comprises no more than 1, 2 or 3 mismatch(es) when hybridized to the targeted RNA.

12. The asdDNA molecule of any one of claims 1 to 11, wherein the first strand has a length selected from the group consisting of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 and 50 nucleotide monomers.

13. The asdDNA molecule of any one of claims 1 to 11, wherein the first strand has a length selected from the group consisting of:
a) 8-50 nucleotide monomers,
b) 10-36 nucleotide monomers,
c) 12-36 nucleotide monomers, and
d) 12-25 nucleotide monomers.

14. The asdDNA molecule of any one of claims 1 to 11, wherein the second strand comprises a substantially complementary region that is at least 70%, 75%, 80%, 85%, 90%, 95% complementary or fully complementary to at least one region of the first strand.

15. The asdDNA molecule of claim 14, wherein the second strand comprises 1, 2, 3 or more mismatch(es) upon forming a complementary duplex to the at least one region of the first strand.

16. The asdDNA molecule of claim 15, wherein the mismatched monomer(s) in the sense strand has a nucleobase selected from the group consisting of A, G, C, and T.

17. The asdDNA molecule of claim 14, wherein the second strand is shorter than the first strand by at least a number of monomers selected from the group consisting of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 and 38.

18. The asdDNA molecule of claim 14, wherein the second strand has a length selected from the group consisting of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, and 36 nucleotide monomers.

19. The asdDNA molecule of claim 14, wherein the second strand has a length of any number of nucleotide monomers that is fewer than that of the first strand, provided that a duplex can be formed with the first strand.

20. The asdDNA molecule of claim 14, wherein at least one of the first base and the last base of the second strand is complementary to a nucleobase in the first strand.

21. The asdDNA molecule of any one of claims 14 to 20, wherein the second strand has a length selected from the group consisting of:
a) 6-36 nucleotide monomers,
b) 6-32 nucleotide monomers,
c) 8-25 nucleotide monomers and
d) 8-23 nucleotide monomers.

22. The asdDNA molecule of any one of claims 1 to 21, wherein the two ends of the first strand are selected from the group consisting of:
a) a 3′-overhang and a 5′-overhang,
b) a 3′-overhang and a blunt end at 5′ end,
c) a 5′-overhang and a blunt end at 3′ end,
d) a 3′-overhang and a 5′-recessed-end, and
e) a 5′-overhang and a 3′-recessed-end.

23. The asdDNA molecule of claim 22, wherein the 3′-overhang of the first strand has a length selected from the group consisting of:
a) 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide monomers,
b) 1-15 nucleotide monomers,
c) 1-10 nucleotide monomers,
d) 1-8 nucleotide monomers, and
e) 1-5 nucleotide monomers.

24. The asdDNA molecule of claim 22, wherein the 5′-overhang of the first strand has a length selected from the group consisting of:
a) 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotide monomers,
b) 1-15 nucleotide monomers,
c) 1-10 nucleotide monomers,
d) 1-8 nucleotide monomers, and
e) 1-5 nucleotide monomers.

25. The asdDNA molecule of claim 22, wherein the first strand has a 3′-overhang of 1-15 nucleotide monomers and a 5′-overhang of 1-15 nucleotide monomers.

26. The asdDNA molecule of claim 22, wherein the first strand has a 3′-overhang of 1-28 nucleotide monomers and a 5′ blunt end or a 5′ recessed end.

27. The asdDNA molecule of claim 22, wherein the first strand has a 5′-overhang of 1-28 nucleotide monomers and a 3′ blunt end or a 3′ recessed end.

28. The asdDNA molecule of any one of claims 1 to 27, wherein at least one nucleotide monomer is a modified nucleotide or nucleotide analogue.

29. The asdDNA molecule of claim 28, wherein the modified nucleotide or nucleotide analogue is a sugar-, backbone-, and/or base-modified nucleotide.

30. The asdDNA molecule of claim 29, wherein the backbone-modified nucleotide has a modification in an internucleoside linkage.

31. The asdDNA molecule of claim 30, wherein the internucleoside linkage is modified to include at least one of a nitrogen or sulphur heteroatom.

32. The asdDNA molecule of claim 31, wherein the modified internucleoside linkage is selected from the group consisting of phosphorothioate (P═S) group, phosphotriesters, methylphosphonates, and phosphoramidate.

33. The asdDNA molecule of claim 28, wherein the first strand and/or the second strand comprises at least one modified internucleoside linkage, and wherein the modified internucleoside linkage is a phosphorothioate internucleoside linkage.

34. The asdDNA molecule of claim 33, wherein each internucleoside linkage of the first strand and/or the second strand is a phosphorothioate internucleoside linkage.

35. The asdDNA molecule of claim 28, wherein the modified nucleotide or nucleotide analogue comprises a modified sugar moiety.

36. The asdDNA molecule of claim 35, wherein the 2′ position of the modified sugar moiety is replaced by a group selected from the group consisting of OR, R, halo, SH, SR, NH₂, NHR, NR₂, and CN, where each R is independently C₁-C₆ alkyl, alkenyl or alkynyl, and halo is F, Cl, Br or I.

37. The asdDNA molecule of claim 35, wherein the 2′ position of the modified sugar moiety is replaced by a group selected from the group consisting of allyl, amino, azido, thio, O-allyl, O-C₁-C₁₀ alkyl, OCF₃, OCH₂F, O(CH₂)₂SCH₃, O(CH₂)₂—O—N(Rₘ)(Rₙ), O—CH₂—C(—O)—N(Rₘ)(Rₙ), and O—CH₂—C(═O)—N(Rₗ)—(CH₂)₂—N(Rₘ)(Rₙ), where each of Rₗ, Rₘ and Rₙ is, independently, H or substituted or unsubstituted C₁-C₁₀ alkyl.

38. The asdDNA molecule of claim 35, the modified sugar moiety is selected from the group consisting of 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups.

39. The asdDNA molecule of claim 35, wherein the modified sugar moiety is substituted by a bicyclic sugar selected from the group consisting of 4′-(CH₂)—O-2′ (LNA); 4′-(CH₂)—S-2; 4′-(CH₂)₂—O-2′(ENA); 4′-CH(CH₃)O-2′(cEt) and 4′-CH(CH₂OCH₃)—O-2′, 4′-C(CH₃)(CH₃)—O-2′, 4′-CH₂—N(OCH₃)-2′, 4′-CH₂—O—N(CH₃)-2′, 4′-CH₂—N(R)—O-2′ (where R is H, C₁-C₁₂ alkyl, or a protecting group), 4′-CH₂—C(H)(CH₃)-2′, and 4′-CH₂—C—(═CH₂)-2′.

40. The asdDNA molecule of claim 35, wherein the modified sugar moiety is selected from the group consisting of 2′-O-methoxyethyl modified sugar (MOE), a 4′-(CH₂)—O-2′ bicyclic sugar (LNA), 2′-deoxy-2′-fluoroarabinose (FANA), and a methyl(methyleneoxy) (4′-CH(CH₃)—O-2) bicyclic sugar (cEt).

41. The asdDNA molecule of claim 28, wherein the modified nucleotide or nucleotide analogue comprises a modified nucleobase.

42. The asdDNA molecule of claim 41, wherein the modified nucleobase is selected from the group consisting of 5-methylcytosine (5-Me-C), inosine base, a tritylated base, 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 1-methyl-pseudo-uracil, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, and 7-deazaguanine and 7-deazaadenine and 3-deazaguanine and 3-deazaadenine.

43. The asdDNA molecule of claim 41, wherein the modified nucleobase is a 5-methylcytosine.

44. The asdDNA molecule of claim 41, wherein each cytosine base is 5-methylcytosine.

45. The asdDNA molecule of any one of claims 1 to 44, wherein the asdDNA is used for modulating gene expression or function in a cell.

46. The asdDNA molecule of any one of claims 1 to 45, wherein the asdDNA molecule is more potent or more efficacious at silencing the target RNA than a corresponding single-stranded antisense oligonucleotide.

47. The asdDNA molecule of any one of claims 1 to 46, wherein the asdDNA molecule is used for modulating gene expression or function in a cell.

48. The asdDNA molecule of claim 47, wherein the cell is a eukaryotic cell.

49. The asdDNA molecule of claim 48, wherein the eukaryotic cell is a mammalian cell.

50. The asdDNA molecule of claim 1, wherein the targeted RNA is either mRNA or non-coding RNA where such RNA either encodes a protein or regulates a part of a biological pathway implicated in a disease.

51. The asdDNA molecule of claim 1, wherein the targeted RNA is selected from the group consisting of:
a) an mRNA of a gene implicated in human or animal diseases or conditions,
b) an mRNA of a gene of a pathogenic microorganism,
c) a viral RNA, and
d) an RNA implicated in a disease or disorder selected from the group consisting of autoimmune diseases, inflammatory diseases, degenerative diseases, infectious diseases, proliferative diseases, metabolic diseases, immune-mediated disorders, allergic diseases, dermatological diseases, malignant diseases, gastrointestinal disorders, respiratory disorders, cardiovascular disorders, renal disorders, rheumatoid disorders, neurological disorders, endocrine disorders, and aging related disorders.

52. The asdDNA molecule of any one of claims 1 to 51, wherein the first strand and/or the second strand is conjugated to a ligand or a moiety.

53. The asdDNA molecule of claim 52, wherein the ligand or moiety is selected from the group consisting of peptide/protein, antibody, polymer, polysaccharide, lipid, hydrophobic moiety or molecule, cationic moiety or molecule, lipophilic compound or moiety oligonucleotide, cholesterol, GalNAc and aptamer.

54. A pharmaceutical composition comprises an asdDNA molecule of any of claims 1-53 as active agent and a pharmaceutically acceptable excipient, carrier, or diluent.

55. The pharmaceutical composition of claim 54, wherein the carrier is selected from the group consisting of a pharmaceutical carrier, a positive-charge carrier, a lipid nanoparticle, a liposome, a protein carrier, a hydrophobic moiety or molecule, a cationic moiety or molecule, GalNAc, a polysaccharide a polymer, a nanoparticle, a nanoemulsion, a cholesterol, a lipid, a lipophilic compound or moiety, and a lipoid.

56. A method for treating or preventing a disease or a condition, wherein the method comprises administering a therapeutically effective amount of the asdDNA molecule of any one of claims 1-53 or the pharmaceutical composition of either claim 54 or claim 55 to a subject in need thereof.

57. The method of claim 56, wherein the disease or condition is selected from the group consisting of cancer, autoimmune disease, inflammatory diseases, degenerative diseases, infectious diseases, proliferative diseases, metabolic diseases, immune-mediated disorders, allergic diseases, dermatological diseases, malignant diseases, gastrointestinal disorders, hepatic disorders, respiratory disorders, cardiovascular disorders, dermatological disorders, renal disorders, rheumatoid disorders, neurological disorders, psychiatric disorders, endocrine disorders, and aging-related disorders or diseases.

58. The method of claim 57, wherein the asdDNA molecule or pharmaceutical composition is administered via a route selected from the group consisting of intravenous injection (iv), subcutaneous injection (sc), per os (po), intramuscular (im) injection, oral administration, inhalation, topical, intrathecal, and other regional administrations.

59. A method for modulating a gene expression or gene function in a eukaryotic cell, wherein the method comprises contacting the cell with an effective amount of the asdDNA molecule of any one of claims 1-53 or the pharmaceutical composition of either claim 54 or 55.

60. An asymmetric short duplex DNA (asdDNA) molecule comprising a first strand and a second strand each comprising linked nucleotide monomers selected from the group consisting of nucleotides, analogs thereof, and modified nucleotides,
wherein the first strand is longer than the second strand by a number of monomers selected from the group consisting of 1, 2, 3, 4, 5, 6, 7, 8, 9 and 10 monomers,
wherein the first strand is substantially complementary to a targeted segment of a targeted RNA through at least one targeting region, and wherein the first strand consists of 10-36 (both range endpoints included) nucleoside monomers linked through a linkage selected from the group consisting of a phosphorothioate linkage, a phosphodiester linkage, or a mixture of phosphorothioate and phosphodiester linkages between adjacent monomers,
wherein the second strand is substantially complementary to the first strand, and forms at least one double-stranded region with the first strand, and wherein the second strand consists of 8-32 (both range endpoints included) nucleoside monomers linked through a linkage selected from the group consisting of a phosphorothioate linkage, a phosphodiester linkage, or a mixture of phosphorothioate and phosphodiester linkages between adjacent monomers,
wherein the asdDNA molecule comprises at least one interspersed segment of ribonucleotide monomers (ISR) linked to at least one deoxyribonucleotide monomer selected from the group consisting of a deoxyribonucleotide, an analog thereof, and a modified deoxyribonucleotide,
wherein the ISR in the asdDNA molecule comprises at least one ribonucleotide monomer selected from the group consisting of a ribonucleotide, an analog thereof, and a modified ribonucleotide,
wherein the asdDNA molecule is used for modulating a target gene expression or function in a cell,
and wherein the asdDNA molecule is more potent or more efficacious at silencing the expression of the target gene than a corresponding ASO in a cell.
